(19) United States
(12) Patent Application Publication
(10) Pub. No.: US 2026/0125480 A1
(43) Pub. Date: May 7, 2026

(54) Bispecific EGFR/C-Met Antibodies

(71) Applicant: Janssen Biotech, Inc., Horsham, PA (US)

(72) Inventors: Mark Chiu, Paoli, PA (US); Sheri Moores, Phoenixville, PA (US); Joost Neijssen, Ultrecht (NL); Paul Parren, Ultrecht (NL); Janine Schuurman, Ultrecht (NL)

(21) Appl. No.: 19/388,613

(22) Filed: Nov. 13, 2025

Related U.S. Application Data
(63) Continuation of application No. 19/027,363, filed on Jan. 17, 2025, now abandoned.
Application No. 19/027,363 is a continuation of application No. 17/398,294, filed on Aug. 10, 2021, now Pat. No. 12,247,077.
Application No. 17/398,294 is a continuation of application No. 16/697,249, filed on Nov. 27, 2019, now abandoned.
Application No. 16/697,249 is a continuation of application No. 15/989,532, filed on May 25, 2018, now abandoned.
Application No. 15/989,532 is a continuation of application No. 15/616,016, filed on Jun. 7, 2017, now abandoned.
Application No. 15/616,016 is a continuation-in-part of application No. 15/386,195, filed on Dec. 21, 2016, now Pat. No. 9,695,242.
Application No. 15/386,195 is a division of application No. 14/283,257, filed on May 21, 2014, now Pat. No. 9,580,508.
Application No. 14/283,257 is a continuation-in-part of application No. 14/086,588, filed on Nov. 21, 2013, now Pat. No. 9,593,164.
(60) Provisional application No. 61/728,912, filed on Nov. 21, 2012.
(60) Provisional application No. 61/782,550, filed on Mar. 14, 2013.
(60) Provisional application No. 61/809,541, filed on Apr. 8, 2013.
(60) Provisional application No. 61/864,717, filed on Aug. 12, 2013.
(60) Provisional application No. 61/892,797, filed on Oct. 18, 2013.

(51) Int. Cl. C07K 16/28; A61K 31/437; A61K 31/4545; A61K 31/517; A61K 31/5377; A61K 33/243; A61K 39/00; A61K 39/395; A61K 45/06

(52) U.S. Cl. CPC C07K 16/2863; A61K 31/437; A61K 31/4545; A61K 31/517; A61K 31/5377; A61K 33/243; A61K 39/395; A61K 39/3955; A61K 45/06; A61K 2039/505; A61K 2300/00; C07K 2317/31; C07K 2317/73; C07K 2317/76; C07K 2318/20

(57) ABSTRACT

Bispecific EGFR/c-Met antibodies and methods of making and using the molecules.

### CROSS REFERENCE TO RELATED APPLICATIONS

This application is a continuation of U.S. application Ser. No. 19/027,363, filed on 17 Jan. 2025, which is a continuation of U.S. application Ser. No. 17/398,294, filed on 10 Aug. 2021 (now U.S. Pat. No. 12,247,077), which is a continuation of U.S. application Ser. No. 16/697,249, filed on 27 Nov. 2019 (now abandoned), which is a continuation of U.S. application Ser. No. 15/989,532, filed on 25 May 2018 (now abandoned), which is a continuation of U.S. application Ser. No. 15/616,016, filed on 7 Jun. 2017 (now abandoned), which is a continuation-in-part of U.S. application Ser. No. 15/386,195, filed on 21 Dec. 2016 (now U.S. Pat. No. 9,695,242), which is a divisional application of U.S. application Ser. No. 14/283,257, filed on 21 May 2014 (now U.S. Pat. No. 9,580,508), which is a continuation-in-part of U.S. application Ser. No. 14/086,588, filed on 21 Nov. 2013 (now U.S. Pat. No. 9,593,164), which claims the benefit of U.S. Provisional Application No. 61/728,912, filed on 21 Nov. 2012, U.S. Provisional Application No. 61/782,550, filed on 14 Mar. 2013, U.S. Provisional Application No. 61/809,541, filed on 8 Apr. 2013, U.S. Provisional Application No. 61/864,717, filed on 12 Aug. 2013, and U.S. Provisional Application No. 61/892,797, filed on 18 Oct. 2013, the entire contents of which are hereby incorporated by reference in their entireties.

### SEQUENCE LISTING

The contents of the electronic sequence listing (JB15032.xml; Size: 369,845 bytes; and Date of Creation: Apr. 23, 2025) is herein incorporated by reference in its entirety.

### FIELD OF THE INVENTION

The present invention relates to bispecific EGFR/c-Met antibodies and methods of making and using the molecules.

### BACKGROUND OF THE INVENTION

Epidermal growth factor receptor (EGFR, ErbB1 or HER1) is a Type I transmembrane glycoprotein of 170 kDa that is encoded by the c-erbBl proto-oncogene. EGFR is a member of the human epidermal growth factor receptor (HER) family of receptor tyrosine kinases (RTK) which includes HER2 (ErbB2), HER3 (ErbB3) and HER4 (ErbB4). EGFR signaling is initiated by ligand binding followed by induction of conformational change, homodimerization or heterodimerization of the receptor with other ErbB family members, and trans-autophosphorylation of the receptor (Ferguson et al., Annu Rev Biophys, 37: 353-73, 2008), which initiates signal transduction cascades that ultimately affect a wide variety of cellular functions, including cell proliferation and survival. Increases in expression or kinase activity of EGFR have been linked with a range of human cancers, making EGFR an attractive target for therapeutic intervention (Mendelsohn et al., Oncogene 19: 6550-6565, 2000; Grünwald et al., J Natl Cancer Inst 95: 851-67, 2003; Mendelsohn et al., Semin Oncol 33: 369-85, 2006). Increases in both the EGFR gene copy number and protein expression have been associated with favorable responses to the EGFR tyrosine kinase inhibitor, IRESSA® (gefitinib), in non-small cell lung cancer (Hirsch et al., Ann Oncol 18:752-60, 2007).

EGFR therapies include both small molecules and anti-EGFR antibodies, approved for treatment of colorectal cancer, pancreatic cancer, head and neck cancer, and non-small cell lung cancer (NSCLC) (Baselga and Arteaga, J Clin Oncol 23:2445-2459 (20005; Gill et al., J Biol Chem, 259:7755-7760, 1984; Goldstein et al., Clin Cancer Res, 1:1311-1318; 1995; Prewett et al., Clin Cancer Res, 4:2957-2966, 1998).

Efficacy of anti-EGFR therapies may depend on tumor type and EGFR mutation/amplification status in the tumor. Side effects of current therapeutics may include skin toxicity (De Roock et al., Lancet Oncol 11:753-762, 2010; Linardou et al., Nat Rev Clin Oncol, 6: 352-366, 2009; Li and Perez-Soler, Targ Oncol 4: 107-119, 2009). EGFR tyrosine kinase inhibitors (TKI) are commonly used as 2^{nd} line therapies for non-small cell lung cancer (NSCLC), but often stop working within twelve months due to resistance pathways (Riely et al., Clin Cancer Res 12: 839-44, 2006).

c-Met encodes a transmembrane tyrosine kinase receptor. It was first identified as a proto-oncogene in 1984 after it was found that treatment with a carcinogen resulted in a constitutively active fusion protein TPR-MET (Cooper et al., Nature 311:29-33, 1984). Activation of c-Met by its ligand hepatocyte growth factor (HGF) stimulates a plethora of cell processes including growth, motility, invasion, metastasis, epithelial-mesenchymal transition, angiogenesis/wound healing, and tissue regeneration (Christensen et al., Cancer Lett 225:1-26, 2005; Peters and Adjei, Nat Rev Clin Oncol 9:314-26, 2012). c-Met is synthesized as a single chain protein that is proteolytically cleaved into a 50 kDa alpha- and 140 kDa beta-subunits that are linked by a disulphide bond (Ma et al., Cancer and Metastasis Reviews, 22: 309-325, 2003). c-Met is structurally similar to other membrane receptors such as RON and Sea. The exact stoichiometry of HGF:c-Met binding is unclear, but it is generally believed that two HGF molecules bind to two c-Met molecules leading to receptor dimerization and autophosphorylation at tyrosines 1230, 1234, and 1235 (Stamos et al., The EMBO Journal 23: 2325-2335, 2004). Ligand-independent c-Met autophosphorylation can also occur due to gene amplification, mutation or receptor overexpression.

c-Met is frequently amplified, mutated or over-expressed in many types of cancer including gastric, lung, colon, breast, bladder, head and neck, ovarian, prostate, thyroid, pancreatic, and CNS cancers. Missense mutations typically localized to the kinase domain are commonly found in hereditary papillary renal cell carcinomas (PRCC) and in 13% of sporadic PRCCs (Schmidt et al., Oncogene 18: 2343-2350, 1999). c-Met mutations localized to the semaphorin or juxtamembrane domains of c-Met are frequently found in gastric, head and neck, liver, ovarian, NSCLC and thyroid cancers (Ma et al., Cancer and Metastasis Reviews, 22: 309-325, 2003; Sakakura et al., Chromosomes and Cancer, 1999. 24:299-305). c-Met amplification has been detected in brain, colorectal, gastric, and lung cancers, often correlating with disease progression (Ma et al., Cancer and Metastasis Reviews, 22: 309-325, 2003). Up to 4% and 20% of non-small cell lung cancer (NSCLC) and gastric cancers, respectively, exhibit c-Met amplification (Sakakura et al., Chromosomes and Cancer, 1999, 24:299-305: Sierra and Tsao, Therapeutic Advances in Medical Oncology, 3:S21-35, 2011). Even in the absence of gene amplification, c-Met overexpression is frequently observed in lung cancer (Ichimura et al., Jpn J Cancer Res, 87:1063-9, 1996). Moreover, in clinical samples, nearly half of lung adenocarcinomas exhibited high levels of c-Met and HGF, both of which correlated with enhanced tumor growth rate, metastasis and poor prognosis (Sierra and Tsao, Therapeutic Advances in Medical Oncology, 3:S21-35, 2011; Siegfried et al., Ann Thorac Surg 66: 1915-8, 1998).

Nearly 60% of all tumors that become resistant to EGFR tyrosine kinase inhibitors increase c-Met expression, amplify c-Met, or increase c-Met only known ligand, HGF (Turke et al., Cancer Cell, 17:77-88, 2010), suggesting the existence of a compensatory pathway for EGFR through c-Met. c-Met amplification was first identified in cultured cells that became resistant to gefitinib, an EGFR kinase inhibitor, and exhibited enhanced survival through the Her3 pathway (Engelman et al., Science, 316:1039-43, 2007). This was further validated in clinical samples where nine of 43 patients with acquired resistance to either erlotinib or gefitinib exhibited c-Met amplification, compared to only two of 62 untreated patients. Four of the nine treated patients also acquired the EGFR activating mutation, T790M, demonstrating simultaneous resistance pathways (Beat et al., Proc Natl Acad Sci USA, 104:20932-7, 2007).

The individual roles of both EGFR and c-Met in cancer is well established, making these targets attractive for combination therapy. Both receptors signal through the same survival and anti-apoptotic pathways (ERK and AKT); thus, inhibiting the pair in combination may limit the potential for compensatory pathway activation thereby improving overall efficacy. Combination therapies targeting EGFR and c-Met are tested in clinical trials with Tarceva® (erlotinib) in combination with anti-c-Met monovalent antibody for NSCLC (Spigel et al., 2011 ASCO Annual Meeting Proceedings 2011, Journal of Clinical Oncology: Chicago, IL. p. 7505) and Tarceva (erlotinib) in combination with ARQ-197, a small molecule inhibitor of c-Met (Adjei et al., Oncologist, 16:788-99, 2011). Combination therapies or bispecific anti-EGFR/c-Met molecules have been disclosed for example in: Intl. Pat. Publ. Nos. WO2008/127710, WO2009/111691, WO2009/126834, WO2010/039248, WO2010/115551 and U.S. Pat. Publ. No. US2009/0042906.

Current small molecule and large molecule therapeutic approaches to antagonize EGFR and/or c-Met signaling pathways for therapy may be sub-optimal due to possible lack of specificity, potential off-target activity and dose-limiting toxicity that may be encountered with small molecule inhibitors. Typical monospecific bivalent antibodies may result in clustering of membrane bound receptors and unwanted activation of the downstream signaling pathways. Monovalent antibodies having full length heavy chains (half arms) pose significant complexity and cost to the manufacturing process.

Accordingly, the need exists for additional monospecific and bispecific EGFR and/or c-Met inhibitors for both therapeutic and diagnostic purpose.

### SUMMARY OF THE INVENTION

One embodiment of the invention is an isolated bispecific epidermal growth factor receptor (EGFR)/hepatocyte growth factor receptor (c-Met) antibody, comprising:
a first heavy chain (HC1) comprising a HC1 constant domain 3 (HC1 CH3) and a HC1 variable region 1 (VH1);
a second heavy chain (HC2) comprising a HC2 constant domain 3 (HC2 CH3) and a HC2 variable region 2 (VH2);
a first light chain (LC1) comprising a light chain variable region 1 (VL1); and a second light chain (LC2) comprising a light chain variable region 2 (VL2), wherein the VH1 and the VL1 pair to form a first antigen-binding site that specifically binds EGFR, the VH2 and the VL2 pair to form a second antigen-binding site that specifically binds c-Met, the HC1 comprises at least one substitution in the HC1 CH3 and the HC2 comprises at least one substitution in the HC2 CH3, and the substitution in the HC1 CH3 and the substitution in the HC2 CH3 occur at different amino acid residue positions, when residue numbering is according to the EU index.

In other embodiments, the invention provides for bispecific EGFR/c-Met antibodies, wherein the antibody inhibits phosphorylation of extracellular signal-related kinases 1 and 2 (ERK1/2) in NCI-H292, NCI-H1975 or SKMES-1 cell line with an IC₅₀ value that is at least about 10-fold less, at least about 20-fold less, at least about 30-fold less, at least about 40-fold less, at least about 50-fold less or at least about 60-fold less when compared to the IC₅₀ value of inhibition of phosphorylation of ERK1/2 in NCI-H292, NCI-H1975 or SKMES-1 cell lines with a mixture of a control monovalent EGFR antibody comprising a heavy chain 3 (HC3) and a light chain 3 (LC3) and a control monovalent c-Met antibody comprising a heavy chain 4 (HC4) and a light chain 4 (LC4), wherein the HC3 and the HC1, the LC3 and the LC1, the HC4 and the HC2, and the LC4 and the LC2 have identical amino acid sequences, respectively, wherein the phosphorylation of ERK1/2 is measured in whole cell lysates using a sandwich immunoassay using an anti-phosphoERK1/2 antibody as a capture antibody and an antibody binding to unphosphorylated and phosphorylated ERK1/2 conjugated with an electrochemiluminescent compound as a detection antibody.

In other embodiments, the invention provides for bispecific EGFR/c-Met antibodies, wherein the antibody inhibits phosphorylation of protein kinase B (AKT) at Ser473 in NCI-H1975 cell line with an IC₅₀ value that is at least about 70-fold less when compared to the IC₅₀ value of inhibition of phosphorylation of AKT at Ser473 in NCI-H1975 cell line with the mixture of the control monovalent EGFR antibody comprising the HC3 and the LC3 and the control monovalent c-Met antibody comprising the HC4 and the LC4, wherein the HC3 and the HC1, the LC3 and the LC1, the HC4 and the HC2, and the LC4 and the LC2 have identical amino acid sequences, respectively, wherein the phosphorylation of AKT at Ser473 is measured in whole cell lysates using a sandwich immunoassay using an antibody binding to unphosphorylated and phosphorylated AKT as a capture antibody and an anti-phosphoAKT Ser473 antibody conjugated to an electrochemiluminescent compound as a detection antibody.

In other embodiments, the invention provides for bispecific EGFR/c-Met antibodies that bind EGFR of SEQ ID NO: 73 at EGFR residues K489, I491, K467 and S492 and c-Met at residues PEFRDSYPIKYVHAF (SEQ ID NO: 238) and FAQSKPDSAEPMDRSA (SEQ ID NO: 239).

In other embodiments, the invention provides for bispecific EGFR/c-Met antibodies that inhibit growth of NCI-H292 or NCI-H1975 cells with an IC₅₀ value that is at least about 300-fold less, at least about 400-fold less, at least about 500-fold less, at least about 600-fold less, at least about 700-fold less or at least about 800-fold less when compared to the IC₅₀ value of inhibition of growth of NCI-H292 or NCI-H1975 cells with cetuximab, when NCI-H292 or NCI-H1975 cells are grown in low attachment conditions.

In other embodiments, the invention provides for bispecific EGFR/c-Met antibodies that inhibit growth of HGF-expressing SKMES-1 cell tumor in SCID Beige mice with percentage (%) T/C value of at least 500-fold less on day 36 when compared to cetuximab, when the bispecific antibody and cetuximab are administered at 20 mg/kg dose.

In other embodiments, the invention provides for bispecific EGFR/c-Met antibodies wherein the HC1 CH3 comprises a K409R or a F405L substitution and the HC2 CH3 comprises a K409R or F405L substitution, wherein residue numbering is according to the EU index.

In other embodiments, the invention provides for bispecific EGFR/c-Met antibodies comprising certain heavy and light chain CDR, VH1, VL1, VH2 VL2, HC1, LC1, LC2 and LC2 sequences.

Another embodiment of the invention is an isolated synthetic polynucleotide encoding the HC1, the HC2, the LC1 or the LC2 of the invention.

Another embodiment of the invention is a vector comprising the polynucleotide of the invention.

Another embodiment of the invention is a host cell comprising the vector of the invention.

Another embodiment of the invention is a method of producing the isolated bispecific EGFR/c-Met antibody, comprising:
combining an isolated monospecific bivalent anti-EGFR antibody comprising two heavy chains of SEQ ID NO: 199 and two light chains of SEQ ID NO: 200 and an isolated monospecific bivalent anti-c-Met antibody comprising two heavy chains of SEQ ID NO: 201 and two light chains of SEQ ID NO: 202 in a mixture of about 1:1 molar ratio;
introducing a reducing agent into the mixture;
incubating the mixture about ninety minutes to about six hours;
removing the reducing agent; and
purifying the bispecific EGFR/c-Met antibody that comprises a first heavy chain of SEQ ID NO: 199 and a second heavy chain of SEQ ID NO: 201, a first light chain of SEQ ID NO: 200 and a second light chain of SEQ ID NO: 202, wherein the first heavy chain of SEQ ID NO: 199 pairs with the first light chain of SEQ ID NO: 200 to form the first binding domain that specifically binds EGFR, and the second heavy chain of SEQ ID NO: 201 pairs with the second light chain of SEQ ID NO: 202 to form the second binding domain that specifically binds c-Met.

Another embodiment of the invention is a pharmaceutical composition comprising the bispecific antibody of the invention and a pharmaceutically acceptable carrier.

Another embodiment of the invention is method of treating a subject having cancer, comprising administering a therapeutically effective amount of the bispecific EGFR/c-Met antibody of the invention to a patient in need thereof for a time sufficient to treat the cancer.

Another embodiment of the invention is method of inhibiting growth or proliferation of cells that express EGFR and/or c-Met, comprising contacting the cells with the bispecific antibody of the invention.

Another embodiment of the invention is method of inhibiting growth or metastasis of EGFR and/or c-Met expressing tumor or cancer cells in a subject comprising administering to the subject an effective amount of the bispecific antibody of the invention to inhibit the growth or metastasis of EGFR and/or c-Met expressing tumor or cancer cells.

### BRIEF DESCRIPTION OF THE DRAWINGS

FIGS. 1A and 1B. Amino acid alignment of the EGFR-binding FN3 domains. The BC and FG loops are boxed at residues 22-28 and 75-86 of SEQ ID NO: 18. Some variants include thermal stability improving L17A, N46K and E861 substitutions (residue numbering according to Tencon SEQ ID NO: 1).

FIG. 2. Sequence alignment of the Tencon27 scaffold (SEQ ID NO: 99) and a TCL14 library (SEQ ID NO: 100) having randomized C-CD-F-FG alternative surface. The loop residues are boxed. Loops and strands are indicated above the sequences.

FIGS. 3A and 3B. Sequence alignment of the c-Met-binding FN3 domains. The C loop and the CD strand and the F loop and the FG strand are boxed and span residues 29-43 and 65-81.

FIG. 4. Inhibition of c-Met phosphorylation in NCI-H292 cells pre-treated with monospecific or bispecific FN3 domain containing molecules and stimulated with HGF is shown. Substantial increase in the potency of the bispecific EGFR/c-Met molecule (ECB1) was observed when compared to a monospecific c-Met-binding FN3 domain (P114AR5P74-A5, shown as A5 in the Figure) on its own or in combination with an EGFR-binding FN3 domain (P54AR4-83v2, shown as 83v2 in the Figure).

FIG. 5. Inhibition of EGFR and c-Met phosphorylation in cells pre-treated with monospecific or bispecific FN3 domain containing molecules. In cell lines expressing high levels of EGFR, NCI-H292 (FIG. 5A) and H596 (FIG. 5B), anti-EGFR monospecific and bispecific FN3 domain containing molecules are equally potent at decreasing EGFR phosphorylation. In cell lines expressing low levels of EGFR relative to c-Met, NCI-H441 (FIG. 5C), bispecific EGFR/c-Met molecules improve the potency for inhibition of EGFR phosphorylation compared to the monospecific EGFR-binding FN3 domain alone. In cell lines with low levels ofc-Met, relative to EGFR, NCI-H292 (FIG. 5D) and H596 (FIG. 5E), inhibition of c-Met phosphorylation is significantly potentiated with bispecific EGFR/c-Met molecule, compared to monospecific c-Met-binding FN3 domain only. Molecules used in the study were: bispecific ECB5 (shown as 17-A3 in the Figure), monospecific EGFR-binding FN3 domain P53A1 R5-17 (shown as “17” in the Figure), bispecific EGFR/c-Met molecule ECB3 (shown as 83-H9 in the Figure), and monospecific c-Met binding FN3 domain P114AR7P93-H9 (shown as H9 in the Figure).

FIG. 6. Pharmacodynamic signaling in tumors isolated from mice dosed with bispecific EGFR/c-Met molecules for 6 h or 72 h. All molecules significantly reduced c-Met, EGFR and ERK phosphorylation at 6 h and 72 h, the degree if inhibition was dependent on the affinity of the FN3 domains to EGFR and/or c-Met. Bispecific molecules were generated by joining EGFR-binding FN3 domain with a high (“83” in the Figure is p54AR4-83v2) or medium (“17v2” in the Figure is P53A1R5-17v2) affinity to a c-Met-binding FN3 domain with high (“A3” in the Figure is P114AR7P94-A3) or medium (“A5” in the Figure is P114AR5P74-A5) affinity.

FIG. 7. Plasma (top) and tumor (bottom) accumulation of bispecific EGFR/cMet molecules of variable affinities linked to an albumin binding domain (ABD) are shown 6 h (left) and 72 h (right) after IP dosing. Six hours after dosing, tumor accumulation is maximal in mice dosed with a bispecific molecule harboring a medium affinity EGFR-binding FN3 domain (17v2) or high affinity EGFR binding domain (83v2). The bispecific molecules incorporated high or medium affinity EGFR or c-Met binding FN3 domains as follows: 83v2-A5-ABD (ECB18; high/medium for EGFR/cMet) 83v2-A3-ABD (ECB38; high/high) 17v2-A5 (ECB28; medium/medium) 17v2-A3-ABD (ECB39; medium/high). In the figure, 83v2 refers to p54AR4-83v2; 17v2 refers to p53A1RS-17v2; A3 refers to p114AR7P94-A3 and A5 refers to p114AR5P74-A5.

FIG. 8. H292-HGF tumor xenografts were implanted into SCID Beige mice. When tumors reached an average volume of approximately 80 mm³, mice were dosed three times per week with bispecific EGFR/c-Met molecules (25 mg/kg) or PBS vehicle. All bispecific molecules reduced tumor growth, the tumor growth inhibition (TGI) being dependent on the affinities of the molecules for c-Met and EGFR (high EGFR-high cMet refers to p54AR4-83v2-p114AR7P94-A3 (ECB38); high EGFR-med cMet refers to p54AR4-83v2-p114AR5P74-A5 (ECB18); med EGFR-high cMet refers to p53A1R5-17v2-p114AR7P94-A3 (ECB39); med EGFR-med-cMet refers to p53A1R5-17-p114AR5P74-A5 (ECB28)).

FIG. 9. H292-HGF tumor xenografts were implanted into SCID Beige mice and they were treated with different therapies. The ani-tumor activity of the therapies is shown (bispecific EGFR/c-Met molecule refers to p54AR4-83v2-p114AR7P94-A3-ABD (ECB38); the other therapies are crizotinib, erlotinib, cetuximab, and the combination of crizotinib and erlotinib).

FIG. 10. SKMES-HGF tumor xenografts were implanted into SCID Beige mice and the mice were treated with different therapies. The anti-tumor activity of the therapies is shown as change in tumor size (mm³) over time. The bispecific EGFR/c-Met antibody EM1-mAb was dosed intraperitoneally (i.p.) twice a week at either 20 mg/kg, 5 mg/kg, or 1 mg/kg; cetuximab was dosed i.p. twice a week at 20 mg/kg. Arrows in the figure show the administration days. Numbers after the antibodies indicated the administered dose.

FIG. 11. HCC827 tumor xenografts were implanted into nude mice and the mice were treated with erlotinib or EM1-mAb at indicated doses. EM1-mAb was dosed biweekly and erlotinib once a day for four weeks. Arrows in the figure show the administration days. The anti-tumor activity of the therapies is shown as change in tumor size (mm) over time.

FIG. 12. SNU-5 tumor xenografts were implanted into CB17/SCID mice and the mice were treated with 10 mg/kg cetuximab or 10 mg/kg or 1 mg/kg EM1-mAb. Antibodies were dosed biweekly for four weeks. Arrows in the figure show the administration days. The antitumor activity of the therapies is shown as change in tumor size (mm³) over time.

FIG. 13. H1975-HGF tumor xenografts were implanted into nude mice and the mice were treated with 10 mg/kg cetuximab, 10 mg/kg EM1-mAb, 50 mg/kg erlotinib, 15 mg/kg afatinib, or a combination of 10 mg/kg EM1-mAb and 15 mg/kg afatinib. Antibodies were dosed biweekly and the small molecules once a day for three weeks. Arrows in the figure show the administration days. The anti-tumor activity of the therapies is shown as change in tumor size (mm) over time.

FIG. 14. HCC827-ER 1 tumor xenografts were implanted into nude mice and the mice were treated with 10 mg/kg EM1-mAb, 25 mg/kg erlotinib, or a combination of the two. EM1-mAb was dosed biweekly and erlotinib once a day for 19 days. Arrows in the figure show the administration days. The anti-tumor activity of the therapies is shown as change in tumor size (mm³) over time.

FIG. 15. Average EGFR and c-Met levels in tumor lysates isolated from H1975 HGF tumor xenografts implanted into SCID Beige mice after administration of a single dose of 20 mg/kg EM1-mAb. Receptor levels are shown as % of PBS control at indicated times posttreatment.

FIG. 16. H1975-HGF tumor xenografts were implanted into nude mice and the mice were treated with 10 mg/kg EM1-mAb or 10 mg/kg EM1-mAb variant IgG2 V234A/G237A/P238S/H268A/V309L/A330S/P331S having no Fc receptor binding and lacking effector functions. Antibodies were dosed biweekly at indicated days. The antitumor activity of the therapies is shown as change in tumor size (mm³) over time.

FIG. 17A. Correlation of EGFR surface density and inhibition of EGFR phosphorylation by the bispecific EM-1 mAb. IC₅₀ values for inhibition of EGFR phosphorylation were plotted vs. EGFR molecules on the surface expressed as antibodies bound per cell (ABC). Pearson correlation p=0.00082, r²=0.7820.

FIG. 17B. Correlation of receptor density and inhibition of c-Met phosphorylation by the bispecific EM-1 mAb. IC₅₀ values for inhibition of c-Met phosphorylation were plotted vs. c-Met molecules on the surface expressed as ABC. Pearson correlation p<0.0001, r²=0.9336.

FIG. 18. The bispecific EGFR/c-Met EM-1 mAb induced heterodimerization of EGFR and c-Met assessed in β-galactosidase fragment mediated complementation assay in U-2 OS cells stably expressing EGFR and c-Met β-gal fusion proteins. Control monovalent EGFR- or c-Met antibodies or parental bivalent anti-EGFR or c-Met antibodies 2F8 or 069 had no effect. E1-F405L-gp120-K409R (●), M1-K409R-gp120-F405L (□), a combination of E1-F405L-gp120-K409R and M1-K409R-gp120-F405L (▾), EM-1 bispecific mAb (♦), anti-cMet parental 069 mAb (⋄), anti-EGFR parental 2F8mAb (▴).

FIG. 19A. Inhibition of c-Met phosphorylation in H292 cells relative to a HGF-treated control. The ratio of EGFR to c-Met surface expression in H292 cells was 5.72. Negative control (♦), E1-F405L-gp120-K409R (●), M1-K409R-gp120-F405L (▪), EM-1 bispecific mAb (▴). The dotted lines indicate the levels of the MSD signal from the EGF-treated control without antibody or unstimulated control as shown in the Figure. ug/mL indicates the antibody log concentration in μg/ml.

FIG. 19B. Inhibition of EGFR phosphorylation in H292 cells relative to an EGFR-treated control. Negative control (♦), E1-F405L-gp120-K409R (9), M1-K409R-gp120-F405L (▪), EM-1 bispecific mAb (▴). The dotted lines indicate the levels of the MSD signal from the EGF-treated control without antibody or unstimulated control as shown in the Figure. ug/mL indicates the antibody log concentration in μg/ml.

FIG. 20. Inhibition of EGFR phosphorylation in SNU-5 cells relative to an EGF-treated control. Negative control (♦), E1-F405L-gp120-K409R (●), M1-K409R-gp120-F405L (▪), EM-1 bispecific mAb (▴). The dotted lines indicate the levels of the MSD signal from the negative controls or unstimulated control as shown in the Figure. ug/mL indicates the antibody log concentration in μg/ml.

FIG. 21. Inhibition of ligand-induced receptor phosphorylation in H1993 cells relative to an EGF-treated control. E1-F405L-gp120-K409R (o), M1-K409R-gp120-F405L (▪), EM-1 bispecific mAb (▴), negative control (♦), combination of E1-F405L-gp120-K409R and M1-K409R-gp120-F405L (▾). X-axis indicates the antibody log nM concentration. The dotted lines indicate the levels of the MSD signal from the unstimulated (vehicle) control or EGF-treated control without antibody.

### DETAILED DESCRIPTION OF THE INVENTION

The term “fibronectin type ill (FN3) domain” (FN3 domain) as used herein refers to a domain occurring frequently in proteins including fibronectins, tenascin, intracellular cytoskeletal proteins, cytokine receptors and prokaryotic enzymes (Bork and Doolittle, Proc Nat Acad Sci USA 89:8990-8994, 1992; Meinke et al., J Bacteriol 175:1910-1918, 1993; Watanabe et al., J Biol Chem 265:15659-15665, 1990). Exemplary FN3 domains are the 15 different FN3 domains present in human tenascin C, the 15 different FN3 domains present in human fibronectin (FN), and non-natural synthetic FN3 domains as described for example in U.S. Pat. Publ. No. 2010/0216708. Individual FN3 domains are referred to by domain number and protein name, e.g., the 3^{rd} FN3 domain of tenascin (TN3), or the 10^{th} FN3 domain of fibronectin (FN10).

The term “substituting” or “substituted” or ‘mutating” or “mutated” as used herein refers to altering, deleting or inserting one or more amino acids or nucleotides in a polypeptide or polynucleotide sequence to generate a variant of that sequence.

The term “randomizing” or “randomized” or “diversified” or “diversifying” as used herein refers to making at least one substitution, insertion or deletion in a polynucleotide or polypeptide sequence.

“Variant” as used herein refers to a polypeptide or a polynucleotide that differs from a reference polypeptide or a reference polynucleotide by one or more modifications for example, substitutions, insertions or deletions.

The term “specifically binds” or “specific binding” as used herein refers to the ability of an FN3 domain, a bispecific agent that specifically binds EGFR and c-Met, or a bispecific EGFR/c-Met antibody of the invention to bind to a predetermined antigen with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. Typically the FN3 domain, the bispecific agent that specifically binds EGFR and c-Met or the bispecific EGFR/c-Met antibody of the invention binds to a predetermined antigen (i.e. EGFR or c-Met) with a K_{D} that is at least ten fold less than its K_{D} for a nonspecific antigen (for example BSA or casein) as measured by surface plasmon resonance using for example a Proteon Instrument (BioRad). Thus, the bispecific EGFR/c-Met FN3 domain containing molecule, the bispecific agent that specifically binds EGFR and c-Met or the bispecific EGFR/c-Met antibody of the invention specifically binds to each EGFR and c-Met with a binding affinity (KD) of at least about 1×10⁻⁶ M or less, for example about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. The bispecific EGFR/c-Met FN3 domain containing molecule, the bispecific agent that specifically binds EGFR and c-Met or the bispecific EGFR/c-Met antibody of the invention that specifically binds to a predetermined antigen may, however, have cross-reactivity to other related antigens, for example to the same predetermined antigen from other species (homologs).

The term “library” refers to a collection of variants. The library may be composed of polypeptide or polynucleotide variants.

The term “stability” as used herein refers to the ability of a molecule to maintain a folded state under physiological conditions such that it retains at least one of its normal functional activities, for example, binding to a predetermined antigen such as EGFR or c-Met.

“Epidermal growth factor receptor” or “EGFR” as used here refers to the human EGFR (also known as HER1 or ErbB1 (Ullrich et al., Nature 309:418-425, 1984) having the amino acid sequence shown in SEQ ID NO: 73 and in GenBank accession number NP_005219, as well as naturally-occurring variants thereof. Such variants include well-known EGFRvIII and other alternatively spliced variants (e.g., as identified by SwissProt Accession numbers P00533-1 (wild type; identical to SEQ ID NO: 73 and NP_005219), P00533-2 (F404L/L405S), P00533-3 (628-705: CTGPGLEGCP . . . GEAPNQALLR→PGNESLKAML . . . SVIITASSCH and 706-1210 deleted), P00533-4 (C628S and 629-1210 deleted), variants GlnQ98, R266, K521, 1674, G962, and P988 (Livingston et al., NIEHS-SNPs, environmental genome project, NIEHS ES15478), T790M, L858R/T790M and del(E746, A750).

“EGFR ligand” as used herein encompasses all (e.g., physiological) ligands for EGFR, including EGF, TGFα heparin binding EGF (HB-EGF), amphiregulin (AR), and epiregulin (EPI).

“Epidermal growth factor” (EGF) as used herein refers to the well-known 53 amino acid human EGF having the amino acid sequence shown in SEQ ID NO: 74.

“Hepatocyte growth factor receptor” or “c-Met” as used herein refers to the human c-Met having the amino acid sequence shown in SEQ ID NO: 101 or in GenBank Accession No: NP_001120972 and natural variants thereof.

“Hepatocyte growth factor” (HGF) as used herein refers to the well-known human HGF having the amino acid sequence shown in SEQ ID NO: 102 which is cleaved to form a dimer of an alpha and beta chain linked by a disulfide bond.

“Blocks binding” or “inhibits binding”, as used herein interchangeably refers to the ability of the FN3 domains, the bispecific EGFR/c-Met FN3 domain containing molecule, the bispecific agent that specifically binds EGFR and c-Met or the bispecific EGFR/c-Met antibody of the invention to block or inhibit binding of the EGFR ligand such as EGF to EGFR and/or HGF to c-Met, and encompass both partial and complete blocking/inhibition. The blocking/inhibition of EGFR ligand such as EGF to EGFR and/or HGF to c-Met by the FN3 domains, the bispecific EGFR/c-Met FN3 domain containing molecule, the bispecific agent that specifically binds EGFR and c-Met or the bispecific EGFR/c-Met antibody of the invention reduces partially or completely the normal level of EGFR signaling and/or c-Met signaling when compared to the EGFR ligand binding to EGFR and/or HOF binding to c-Met without blocking or inhibition. The FN3 domains, the bispecific EGFR/c-Met FN3 domain containing molecule, the bispecific agent that specifically binds EGFR and c-Met or the bispecific EGFR/c-Met antibody of the invention “blocks binding” of the EGFR ligand such as EGF to EGFR and/or HGF to c-Met when the inhibition is at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100%. Inhibition of binding can be measured using well known methods, for example by measuring inhibition of binding of biotinylated EGF on EGFR expressing A431 cells exposed to the FN3 domain, the bispecific EGFR/c-Met FN3 domain containing molecule, the bispecific agent that specifically binds EGFR and c-Met or the bispecific EGFR/c-Met antibody of the invention using FACS, and using methods described herein, or measuring inhibition of binding of biotinylated HGF on c-Met extracellular domain using well known methods and methods described herein.

The term “EGFR signaling” refers to signal transduction induced by EGFR ligand binding to EGFR resulting in autophosphorylation of at least one tyrosine residue in the EGFR. An exemplary EGFR ligand is EGF.

“Neutralizes EGFR signaling” as used herein refers to the ability of the FN3 domains, the bispecific EGFR/c-Met FN3 domain containing molecule, the bispecific agent that specifically binds EGFR and c-Met or the bispecific EGFR/c-Met antibody of the invention to inhibit EGFR signaling induced by EGFR ligand such as EGF by at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100%.

The term “c-Met signaling” refers to signal transduction induced by HGF binding to c-Met resulting in autophosphorylation of at least one tyrosine residue in the c-Met. Typically at least one tyrosine residue at positions 1230, 1234, 1235 or 1349 is autophosphorylated upon HGF binding.

“Neutralizes c-Met signaling” as used herein refers to the ability of the FN3 domain, the bispecific EGFR/c-Met FN3 domain containing molecule, the bispecific agent that specifically binds EGFR and c-Met or the bispecific EGFR/c-Met antibody of the invention to inhibit c-Met signaling induced by HGF by at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100%.

“Overexpress”, “overexpressed” and “overexpressing” as used herein interchangeably refer to a cancer or malignant cell that has measurably higher levels of EGFR and/or c-Met on the surface compared to a normal cell of the same tissue type. Such overexpression may be caused by gene amplification or by increased transcription or translation. EGFR and/or c-Met expression and overexpression can be measured using well know assays using for example ELISA, immunofluorescence, flow cytometry or radioimmunoassay on live or lysed cells. Alternatively, or additionally, levels of EGFR and/or c-Met-encoding nucleic acid molecules may be measured in the cell for example using fluorescent in situ hybridization, Southern blotting, or PCR techniques. EGFR and/or c-Met is overexpressed when the level of EGFR and/or c-Met on the surface of the cell is at least 1.5-fold higher when compared to the normal cell.

“Tencon” as used herein refers to the synthetic fibronectin type ill (FN3) domain having the sequence shown in SEQ ID NO: 1 and described in U.S. Pat. Publ. No. US2010/0216708.

A “cancer cell” or a “tumor cell” as used herein refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is exemplified by, e.g., morphological changes, immortalization of cells, aberrant growth control, foci formation, proliferation, malignancy, tumor specific marker levels, invasiveness, tumor growth or suppression in suitable animal hosts such as nude mice, and the like, in vitro, in vWI, and ex vivo (Freshney, Culture of Animal Cells: A Manual of Basic Technique (3rd ed. 1994)).

The term “vector” means a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

The term “expression vector” means a vector that can be utilized in a biological system or in a reconstituted biological system to direct the translation of a polypeptide encoded by a polynucleotide sequence present in the expression vector.

The term “polynucleotide” means a molecule comprising a chain of nucleotides covalently linked by a sugar-phosphate backbone or other equivalent covalent chemistry. Double and single-stranded DNAs and RNAs are typical examples of polynucleotides.

“Complementary DNA” or “cDNA” refers to the well-known synthetic polynucleotide that shares the arrangement of sequence elements found in native mature mRNA species with contiguous exons, with the intervening introns present in genomic DNA are removed. The codons encoding the initiator methionine may or may not be present in cDNA. cDNA may be synthesized for example by reverse transcription or synthetic gene assembly.

“Synthetic” or “non-natural” or “artificial” as used herein refers to a polynucleotide or a polypeptide molecule not present in nature.

The term “polypeptide” or “protein” means a molecule that comprises at least two amino acid residues linked by a peptide bond to form a polypeptide. Small polypeptides of less than about 50 amino acids may be referred to as “peptides”.

The term “bispecific EGFR/c-Met molecule” or “bispecific EGFR/c-Met FN3 domain containing molecule” as used herein refers to a molecule comprising an EGFR binding FN3 domain and a distinct c-Met binding FN3 domain that are covalently linked together either directly or via a linker. An exemplary bispecific EGFR/c-Met binding molecule comprises a first FN3 domain specifically binding EGFR and a second FN3 domain specifically binding c-Met.

“Valent” as used herein refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Mixture” as used herein refers to a sample or preparation of two or more FN3 domains not covalently linked together. A mixture may consist of two or more identical FN3 domains or distinct FN3 domains. Mixture as used herein also refers to a sample or preparation of two or more monovalent antibodies that are monovalent towards EGFR and/or monovalent towards c-Met.

The term “bispecific agent that specifically binds EGFR and c-Met” as used herein refers to a molecule comprising a first domain that specifically binds EGFR and a second domain that specifically binds c-Met. An exemplary agent that specifically binds EGFR and c-Met is a bispecific antibody. Another exemplary bispecific agent that specifically binds EGFR and c-Met is a molecule comprising an EGFR binding FN3 domain and a distinct c-Met binding FN3 domain. The bispecific agent that specifically binds EGFR and c-Met may be composed of a single polypeptide or more than one polypeptide.

The term “bispecific anti-EGFR/c-Met antibody” or “bispecific EGFR/c-Met antibody” as used herein refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

The term “antibodies” as used herein is meant in a broad sense and includes immunoglobulin molecules including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄. Antibody light chains of any vertebrate species can be assigned to one of two clearly distinct types, namely kappa (κ) and lambda (λ), based on the amino acid sequences of their constant domains.

The term “antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (VH), or a light chain variable region (VL). Antibody fragments include a Fab fragment, a monovalent fragment consisting of the VL, VH, CL and CH1 domains: a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a domain antibody (dAb) fragment (Ward et al (1989) Nature 341:544-546), which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in PCT Intl. Publ. Nos. WO1998/44001, WO1988/01649, WO1994/13804, and WO1992/01047. These antibody fragments are obtained using well known techniques known to those of skill in the art, and the fragments are screened for utility in the same manner as are full length antibodies.

The phrase “isolated antibody” refers to an antibody or antibody fragment that is substantially free of other antibodies having different antigenic specificities (e.g., an isolated bispecific antibody specifically binding EGFR and c-Met is substantially free of antibodies that specifically bind antigens other than human EGFR and c-Met). An isolated antibody that specifically binds EGFR and c-Met, however, can have cross-reactivity to other antigens, such as orthologs of human EGFR and/or c-Met, such as Macaca fascicularis (cynomolgus) EGFR and/or c-Met. Moreover, an isolated antibody may be substantially free of other cellular material and/or chemicals.

An antibody variable region consists of a “framework” region interrupted by three “antigen binding sites”. The antigen binding sites are defined using various terms: (i) Complementarity Determining Regions (CDRs), three in the VH (HCDR1, HCDR2, HCDR3), and three in the VL (LCDR1, LCDR2, LCDR3), are based on sequence variability (Wu and Kabat (1970) J Exp Med 132:211-50, 1970; Kabat et al Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3), refer to the regions of an antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (http://www_imgt_org) provides a standardized numbering and definition of antigen-binding sites. The correspondence between CDRs, HVs and IMGT delineations is described in Lefranc et al., Dev Comparat Immunol 27:55-77, 2003.

“Chothia residues” as used herein are the antibody VL and VH residues numbered according to Al-Lazikani (Al-Lazikani et al., J Mol Biol 273:927-48, 1997).

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human-adapted” antibodies or “human framework adapted (HFA)” antibodies refers to humanized antibodies adapted according to methods described in U.S. Pat. Publ. No. US2009/0118127. Human-adapted antibodies are humanized by selecting the acceptor human frameworks based on the maximum CDR and FR similarities, length compatibilities and sequence similarities of CDR1 and CDR2 loops and a portion of light chain CDR3 loops.

“Human antibody” refers to an antibody having heavy and light chain variable regions in which both the framework and the antigen binding sites are derived from sequences of human origin. If the antibody contains a constant region, the constant region also is derived from sequences of human origin.

Human antibody comprises heavy or light chain variable regions that are “derived from” sequences of human origin if the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. “Human antibody” may contain amino acid differences when compared to the human germline or rearranged immunoglobulin sequences due to for example naturally occurring somatic mutations or intentional introduction of substitutions in the framework or antigen binding sites. Typically, “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene. In some cases, “human antibody” may contain consensus framework sequences derived from human framework sequence analyses, for example as described in Knappik et al., J Mol Biol 296:57-86, 2000), or synthetic HCDR3 incorporated into human immunoglobulin gene libraries displayed on phage, for example as described in Shi et al., J Mol Biol 397:385-96, 2010 and Intl. Pat. Publ. No. WO2009/085462). Antibodies in which antigen binding sites are derived from a non-human species are not included in the definition of “human antibody”.

Isolated humanized antibodies may be synthetic. Human antibodies, while derived from human immunoglobulin sequences, may be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

The term “recombinant antibody” as used herein, includes all antibodies that are prepared, expressed, created or isolated by recombinant means, such as antibodies isolated from an animal (e.g., a mouse) that is transgenic or transchromosomal for human immunoglobulin genes or a hybridoma prepared therefrom (described further below), antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, or antibodies that are generated in vitro using Fab arm exchange.

The term “monoclonal antibody” as used herein refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes.

The term “substantially identical” as used herein means that the two antibody variable region amino acid sequences being compared are identical or have “insubstantial differences”. Insubstantial differences are substitutions of 1, 2, 3, 4, 5, 6, 7, 8, 9, 10, 11, 12, 13, 14, or 15 amino acids in an antibody variable region sequence that do not adversely affect antibody properties. Amino acid sequences substantially identical to the variable region sequences disclosed herein are within the scope of the invention. In some embodiments, the sequence identity can be about 90%, 91%, 92%, 93%, 94%, 95%, 96%, 97%, 98%, 99% or higher. Percent identity can be determined for example by pairwise alignment using the default settings of the AlignX module of Vector NTI v.9.0.0 (Invitrogen, Carlsbad, CA). The protein sequences of the present invention can be used as a query sequence to perform a search against public or patent databases to, for example, identify related sequences. Exemplary programs used to perform such searches are the XBLAST or BLASTP programs (http_//www_ncbi_nlm/nih_gov), or the GenomeQuest (GenomeQuest, Westborough, MA) suite using the default settings.

The term “epitope” as used herein means a portion of an antigen to which an antibody specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

The term “in combination with” as used herein means that two or more therapeutics can be administered to a subject together in a mixture, concurrently as single agents or sequentially as single agents in any order.

The numbering of amino acid residues in the antibody constant region throughout the specification is performed according to the EU index as described in Kabat et al., Sequences of Proteins of Immunological Interest, 5th Ed. Public Health Service, National Institutes of Health, Bethesda, MD. (1991), unless otherwise explicitly stated.

### Compositions of Matter

The present invention provides bispecific agents that specifically bind EGFR and c-Met. The present invention provides polypeptides and polynucleotides encoding the bispecific agents of the invention or complementary nucleic acids thereof, vectors, host cells, and methods of making and using them.

Monospecific and Bispecific EGFR and/or c-Met FN3 Domain Containing Binding Molecules

### Monospecific EGFR FN3 Domain Containing Binding Molecules

The present invention provides fibronectin type III (FN3) domains that bind specifically to epidermal growth factor receptor (EGFR) and block binding of epidermal growth factor (EGF) to EGFR, and thus can be widely used in therapeutic and diagnostic applications. The present invention provides polynucleotides encoding the FN3 domains of the invention or complementary nucleic acids thereof, vectors, host cells, and methods of making and using them.

The FN3 domains of the invention bind EGFR with high affinity and inhibit EGFR signaling, and may provide a benefit in terms of specificity and reduced off-target toxicity when compared to small molecule EGFR inhibitors, and improved tissue penetration when compared to conventional antibody therapeutics.

One embodiment of the invention is an isolated fibronectin type III (FN3) domain that specifically binds epidermal growth factor receptor (EGFR) and blocks binding of epidermal growth factor (EGF) to EGFR.

The FN3 domains of the invention may block EGF binding to the EGFR with an IC₅₀ value of less than about 1×10⁻⁷ M, less than about 1×10⁻⁸ M, less than about 1×10⁻⁹ M, less than about 1×10⁻¹⁰ M, less than about 1×10⁻¹¹ M, or less than about 1×10⁻¹² M in a competition assay employing A431 cells and detecting amount of fluorescence from bound biotinylated EGF using streptavidin-phycoerythrin conjugate at 600 nM on A431 cells incubated with or without the FN3 domains of the invention. Exemplary FN3 domains may block EGF binding to the EGFR with an IC₅₀ value between about 1×10⁻⁹ M to about 1×10⁻⁷ M, such as EGFR binding FN3 domains having the amino acid sequence of SEQ ID NOs: 18-29, 107-110, or 122-137. The FN3 domains of the invention may block EGF binding to the EGFR by at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% when compared to binding of EGF to the EGFR in the absence of the FN3 domains of the invention using the same assay conditions.

The FN3 domain of the invention may inhibit EGFR signaling by at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% when compared to the level of signaling in the absence of the FN3 domains of the invention using the same assay conditions.

Binding of a ligand such as EGF to EGFR stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division. Inhibition of EGFR signaling may result in inhibition in one or more EGFR downstream signaling pathways and therefore neutralizing EGFR may have various effects, including inhibition of cell proliferation and differentiation, angiogenesis, cell motility and metastasis.

EGFR signaling may be measured using various well know methods, for example measuring the autophosphorylation of the receptor at any of the tyrosines Y1068, Y1148, and Y1173 (Downward et al., Nature 311:483-5, 1984) and/or phosphorylation of natural or synthetic substrates. Phosphorylation can be detected using well known methods such as an ELISA assay or a western plot using a phosphotyrosine specific antibody. Exemplary assays can be found in Panek et al., J Pharmacol Exp Thera 283:1433-44, 1997 and Batley et al., Life Sci 62:143-50, 1998, and assays described herein.

In one embodiment, the FN3 domain of the invention inhibits EGF-induced EGFR phosphorylation at EGFR residue position Tyrosine 1173 with an IC₅₀ value of less than about 2.5×10⁻⁶ M, for example less than about 1×10⁻⁶ M, less than about 1×10⁻⁷ M, less than about 1×10⁻⁸ M, less than about 1×10⁻⁹ M, less than about 1×10⁻¹⁰ M, less than about 1×10⁻¹¹ M, or less than about 1×10⁻¹² M when measured in A431 cells using 50 ng/mL human EGF.

In one embodiment, the FN3 domain of the invention inhibits EGF-induced EGFR phosphorylation at EGFR residue position Tyrosine 1173 with an IC₅₀ value between about 1.8×10⁻⁸ M to about 2.5×10⁻⁶ M when measured in A431 cells using 50 ng/mL human EGF. Such exemplary FN3 domains are those having the amino acid sequence of SEQ ID NOs: 18-29, 107-110, or 122-137.

In one embodiment, the FN3 domain of the invention binds human EGFR with a dissociation constant (K_{D}) of less than about 1×10⁻⁸ M, for example less than about 1×10⁻⁹ M, less than about 1×10⁻¹⁰ M, less than about 1×10⁻¹¹ M, less than about 1×10⁻¹² M, or less than about 1×10⁻¹³ M as determined by surface plasmon resonance or the Kinexa method, as practiced by those of skill in the art. In some embodiments, the FN3 domain of the invention binds human EGFR with a K_{D} of between about 2×10⁻¹⁰ to about 1×10⁻⁸ M. The affinity of a FN3 domain for EGFR can be determined experimentally using any suitable method. (See, for example, Berzofsky, et al., “Antibody-Antigen Interactions,” In Fundamental Immunology, Paul, W. E., Ed., Raven Press: New York, NY (1984); Kuby, Janis Immunology, W. H. Freeman and Company: New York, NY (1992); and methods described herein). The measured affinity of a particular FN3 domain-antigen interaction can vary if measured under different conditions (e.g., osmolarity, pH). Thus, measurements of affinity and other antigen-binding parameters (e.g., K_{D}, Kₒₙ, K_{off}) are preferably made with standardized solutions of protein scaffold and antigen, and a standardized buffer, such as the buffer described herein.

Exemplary FN3 domains of the invention that bind EGFR include FN3 domains of SEQ ID NOs: 18-29, 107-110, or 122-137.

In one embodiment, the FN3 domain that specifically binds EGFR comprises an amino acid sequence at least 87% identical to the amino acid sequence of SEQ ID NO: 27.

In one embodiment, the FN3 domain that specifically binds EGFR comprises
an FG loop comprising the sequence HNVYKDTNX₉RGL (SEQ ID NO: 179) or the sequence LGSYVFEHDVML (SEQ ID NO: 180), wherein X₉ is M or I; and
a BC loop comprising the sequence X₁X₂X₃X₄X₅X₆X₇X₈ (SEQ ID NO: 181);
wherein
   X₁ is A, T, G or D;
   X₂ is A, D, Y or W;
   X₃ is P, D or N;
   X₄ is L or absent;
   X₅ is D, H, R, G, Yor W;
   X₆ is G, D or A;
   X₇ is A, F, G, H or D; and
   X₈ is Y, F or L.

The FN3 domains of the invention that specifically bind EGFR and inhibit autophosphorylation of EGFR may comprise as a structural feature an FG loop comprising the sequence HNVYKDTNX₉RGL (SEQ ID NO: 179) or the sequence LGSYVFEHDVML (SEQ ID NO: 180), wherein X₉ is M or I. Such FN3 domains may further comprise a BC loop of 8 or 9 amino acids in length and defined by the sequence X₁X₂X₃X₄X₅X₆X₇X₈(SEQ ID NO: 181), and inhibit EGFR autophosphorylation with an IC₅₀ value of less than about 2.5×10⁻⁶ M, or with an IC₅₀ value of between about 1.8×10⁻⁸ M to about 2.5×10⁻⁶ M when measured in A431 cells using 50 ng/mL human EGF.

The FN3 domains of the invention that specifically bind EGFR and inhibit autophosphorylation of EGFR further comprise the sequence of

(SEQ ID NO: 182)
LPAPKNLVVSEVTEDSLRLSWX₁X₂X₃X₄X₅X₆X₇X₈DSFLIQYQESEKV
GEAINLTVPGSERSYDLTGLKPGTEYTVSIYGVHNVYKDTNX₉RGLP
LSAEFTT,

wherein

(SEQ ID NO: 183)
LPAPKNLVVSEVTEDSLRLSWX₁X₂X₃X₄X₅X₆X₇X₈DSFLIQYQESEKV
GEAINLTVPGSERSYDLTGLKPGTEYTVSIYGVLGSYVFEHDVMLPL
SAEFTT,

X₁ is A, T, G or D;
X₂ is A, D, Y or W;
X₃ is P, D or N;
X₄ is L or absent;
X₅ is D, H, R, G, Y or W;
X₆ is G, D or A;
X₇ is A, F, G, H or D;
X₈ is Y, F or L; and
X₉ is M or I

The EGFR binding FN3 domains can be generated and tested for their ability to inhibit EGFR autophosphorylation using well known methods and methods described herein.

Another embodiment of the invention is an isolated FN3 domain that specifically binds EGFR, wherein the FN3 domain comprises the sequence shown in SEQ ID NOs: 18-29, 107-110, or 122-137.

In some embodiments, the EGFR binding FN3 domains comprise an initiator methionine (Met) linked to the N-terminus or a cysteine (Cys) linked to a C-terminus of a particular FN3 domain, for example to facilitate expression and/or conjugation of half-life extending molecules.

Another embodiment of the invention is an isolated fibronectin type III (FN3) domain that specifically binds EGFR and blocks binding of EGF to the EGFR, wherein the FN3 domain is isolated from a library designed based on Tencon sequence of SEQ ID NO: 1.

Monospecific c-Met FN3 Domain Containing Binding Molecules

The present invention provides fibronectin type III (FN3) domains that bind specifically to hepatocyte growth factor receptor (c-Met) and block binding of hepatocyte growth factor (HGF) to c-Met, and thus can be widely used in therapeutic and diagnostic applications. The present invention provides polynucleotides encoding the FN3 domains of the invention or complementary nucleic acids thereof, vectors, host cells, and methods of making and using them.

The FN3 domains of the invention bind c-Met with high affinity and inhibit c-Met signaling, and may provide a benefit in terms of specificity and reduced off-target toxicity when compared to small molecule c-Met inhibitors, and improved tissue penetration when compared to conventional antibody therapeutics. The FN3 domains of the invention are monovalent, therefore preventing unwanted receptor clustering and activation that may occur with other bivalent molecules.

One embodiment of the invention is an isolated fibronectin type III (FN3) domain that specifically binds hepatocyte growth factor receptor (c-Met) and blocks binding of hepatocyte growth factor (HGF) to c-Met.

The FN3 domains of the invention may block HGF binding to c-Met with an IC₅₀ value of less than about 1×10⁻⁷ M, less than about 1×10⁻⁸ M, less than about 1×10⁻⁹ M, less than about 1×10⁻¹⁰ M, less than about 1×10⁻¹¹ M, or less than about 1×10⁻¹² M in an assay detecting inhibition of binding of biotinylated HGF to c-Met-Fc fusion protein in the presence of the FN3 domains of the invention. Exemplary FN3 domains may block HGF binding to the c-Met with an IC₅₀ value between about 2×10⁻¹⁰ M to about 6×10⁻⁸ M. The FN3 domains of the invention may block HGF binding to c-Met by at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% when compared to binding of HGF to c-Met in the absence of the FN3 domains of the invention using the same assay conditions.

The FN3 domain of the invention may inhibit c-Met signaling by at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% when compared to the level of signaling in the absence of FN3 domains of the invention using the same assay conditions.

Binding of HGF to c-Met stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division. Inhibition ofc-Met signaling may result in inhibition of one or more c-Met downstream signaling pathways and therefore neutralizing c-Met may have various effects, including inhibition of cell proliferation and differentiation, angiogenesis, cell motility and metastasis.

c-Met signaling may be measured using various well know methods, for example measuring the autophosphorylation of the receptor on at least one tyrosine residues Y1230, Y1234, Y1235 or Y1349 and/or phosphorylation of natural or synthetic substrates. Phosphorylation may be detected, for example, using an antibody specific for phosphotyrosine in an ELISA assay or on a western blot. Assays for tyrosine kinase activity are described for example in: Panek et al., J Pharmacol Exp Thera 283:1433-44, 1997 and Batley et al., Life Sci 62:143-50, 1998, and assays described herein.

In one embodiment, the FN3 domain of the invention inhibits HGF-induced c-Met phosphorylation at c-Met residue position 1349 with an IC₅₀ value of less than about 1×10⁻⁶ M, less than about 1×10⁻⁷ M, less than about 1×10⁻⁸ M, less than about 1×10⁻⁹ M, less than about 1×10⁻¹⁰ M, less than about 1×10⁻¹¹ M, or less than about 1×10⁻¹² M when measured in NCI-H441 cells using 100 ng/mL recombinant human HGF.

In one embodiment, the FN3 domain of the invention inhibits HGF-induced c-Met phosphorylation at c-Met tyrosine Y1349 with an IC₅₀ value between about 4×10⁻⁹ M to about 1×10⁻⁶ M when measured in NCI-H441 cells using 100 ng/mL recombinant human HGF.

In one embodiment, the FN3 domain of the invention binds human c-Met with an dissociation constant (K_{D}) of equal to or less than about 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M, or 1×10⁻¹⁵ M as determined by surface plasmon resonance or the Kinexa method, as practiced by those of skill in the art. In some embodiments, the FN3 domain of the invention binds human c-Met with a K_{D} of between about 3×10⁻¹⁰ M to about 5×10⁻⁸ M. The affinity of a FN3 domain for c-Met may be determined experimentally using any suitable method. (See, for example, Berzofsky, et al., “Antibody-Antigen Interactions,” In Fundamental Immunology, Paul, W. E., Ed., Raven Press: New York, NY (1984); Kuby, Janis Immunology, W. H. Freeman and Company: New York, NY (1992); and methods described herein). The measured affinity of a particular FN3 domain-antigen interaction can vary if measured under different conditions (e.g., osmolarity, pH). Thus, measurements of affinity and other antigen-binding parameters (e.g., K_{D}, Kₒₙ, K_{off}) are preferably made with standardized solutions of protein scaffold and antigen, and a standardized buffer, such as the buffer described herein.

Exemplary FN3 domains of the invention that bind c-Met include FN3 domains having the amino acid sequence of SEQ ID NOs: 32-49 or 111-114.

In one embodiment, the FN3 domain that specifically binds c-Met comprises an amino acid sequence at least 83% identical to the amino acid sequence of SEQ ID NO: 41.

In one embodiment, the FN3 domain that specifically binds c-Met comprises
a C strand and a CD loop comprising the sequence DSFX₁₀IRYX₁₁E X₁₂X₁₃X₁₄X₁₅GX₁₆ (SEQ ID NO: 184), wherein
   X₁₀ is W, F or V;
   X₁₁ is D, F or L;
   X₁₂ is V, F or L;
   X₁₃ is V, L or T;
   X₁₄ is V, R, G, L, T or S;
   X₁₅ is G, S, A, T or K; and
   X₁₆ is E or D; and
a F strand and a FG loop comprising the sequence TEYX₁₇VX₁₈X₁₉X₂₀V KGGX₂₁X₂₂SX₂₃ (SEQ ID NO: 185), wherein
   X₁₇ is Y, W, IV, G or A;
   X₁₈ is N, T, Q or G;
   X₁₉ is L, M, N or I;
   X₂₀ is G or S;
   X₂₁ is S, L, G, Y, T, R, H or K;
   X₂₂ is I, V or L; and
   X₂₃ is V, T, H, I, P, Y or L.

The FN3 domains of the invention that specifically bind c-Met and inhibit autophosphorylation of c-Met further comprises the sequence:

(SEQ ID NO: 186)
LPAPKNLVVSRVTEDSARLSWTAPDAAFDSFX₁₀IRYX₁₁EX₁₂X₁₃
X₁₄X₁₅GX₁₆AIVLTVPGSERSYDLTGLKPGTEYX₁₇VX₁₈IX₁₉X₂₀VK
GGX₂₁X₂₂SX₂₃PLSAEFTT,

wherein
X₁₀ is W, F or V; and
X₁₁ is D, F or L;
X₁₂ is V, F or L;
X₁₃ is V, L or T;
X₁₄ is V, R, G, L, T or S;
X₁₅ is G, S, A, T or K;
X₁₆ is E or D;
X₁₇ is Y, W, I, V, G or A;
X₁₈ is N, T, Q or G;
X₁₉ is L, M, N or I;
X₂₀ is G or S;
X₂₁ is S, L, G, Y, T, R. H or K;
X₂₂ is I, V or L; and
X₂₃ is V, T, H, I, P, Y or L.

Another embodiment of the invention is an isolated FN3 domain that specifically binds c-Met, wherein the FN3 domain comprises the sequence shown in SEQ ID NOs: 32-49 or 111-114.

Another embodiment of the invention is an isolated fibronectin type III (FN3) domain that specifically binds c-Met and blocks binding of HGF to the c-Met, wherein the FN3 domain is isolated from a library designed based on Tencon sequence of SEQ ID NO: 1.

Isolation of EGFR or c-Met FN3 Domains from a Library Based on Tencon Sequence

Tencon (SEQ ID NO: 1) is a non-naturally occurring fibronectin type III (FN3) domain designed from a consensus sequence of fifteen FN3 domains from human tenascin-C (Jacobs et al., Protein Engineering, Design, and Selection, 25:107-117, 2012; U.S. Pat. Publ. No. 2010/0216708). The crystal structure of Tencon shows six surface-exposed loops that connect seven beta-strands as is characteristic to the FN3 domains, the beta-strands referred to as A, B, C, D, E, F and G, and the loops referred to as AB, BC, CD, DE, EF, and FG loops (Bork and Doolittle, Proc Natl Acad Sci USA 89:8990-8992, 1992; U.S. Pat. No. 6,673,901). These loops, or selected residues within each loop, can be randomized in order to construct libraries of fibronectin type III (FN3) domains that can be used to select novel molecules that bind EGFR or c-Met. Table I shows positions and sequences of each loop and beta-strand in Tencon (SEQ ID NO: 1).

Library designed based on Tencon sequence may thus have randomized FG loop, or randomized BC and FG loops, such as libraries TCL1 or TCL2 as described below. The Tencon BC loop is 7 amino acids long, thus 1, 2, 3, 4, 5, 6 or 7 amino acids may be randomized in the library diversified at the BC loop and designed based on Tencon sequence. The Tencon FG loop is 7 amino acids long, thus 1, 2, 3, 4, 5, 6 or 7 amino acids may be randomized in the library diversified at the FG loop and designed based on Tencon sequence. Further diversity at loops in the Tencon libraries may be achieved by insertion and/or deletions of residues at loops. For example, the FG and/or BC loops may be extended by 1-22 amino acids, or decreased by 1-3 amino acids. The FG loop in Tencon is 7 amino acids long, whereas the corresponding loop in antibody heavy chains ranges from 4-28 residues. To provide maximum diversity, the FG loop may be diversified in sequence as well as in length to correspond to the antibody CDR3 length range of 4-28 residues. For example, the FG loop can further be diversified in length by extending the loop by additional 1, 2, 3, 4 or 5 amino acids.

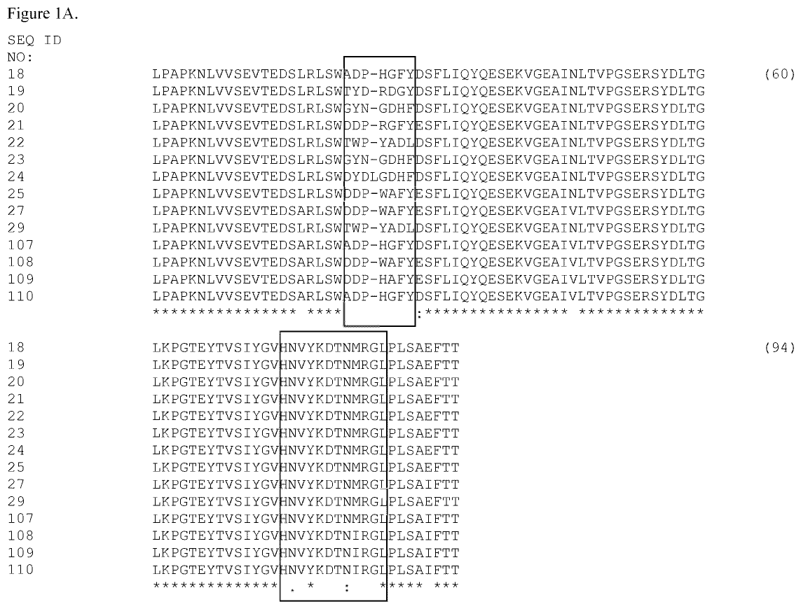

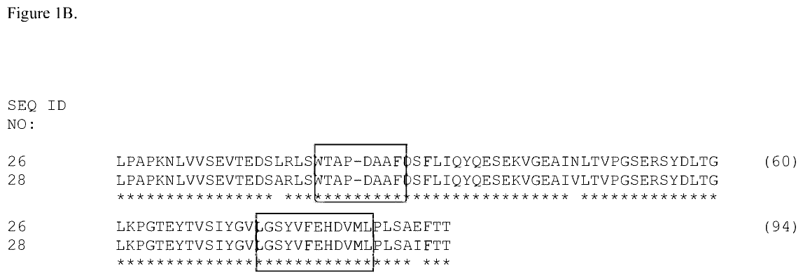

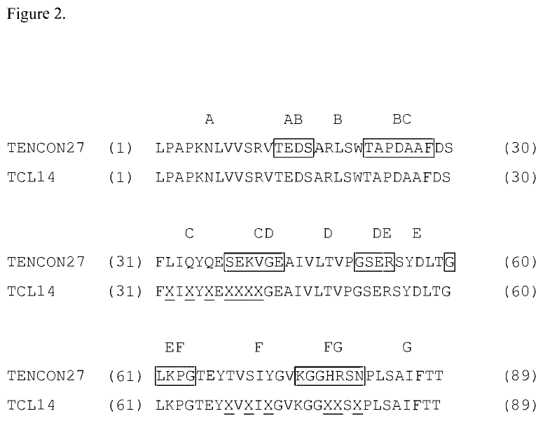

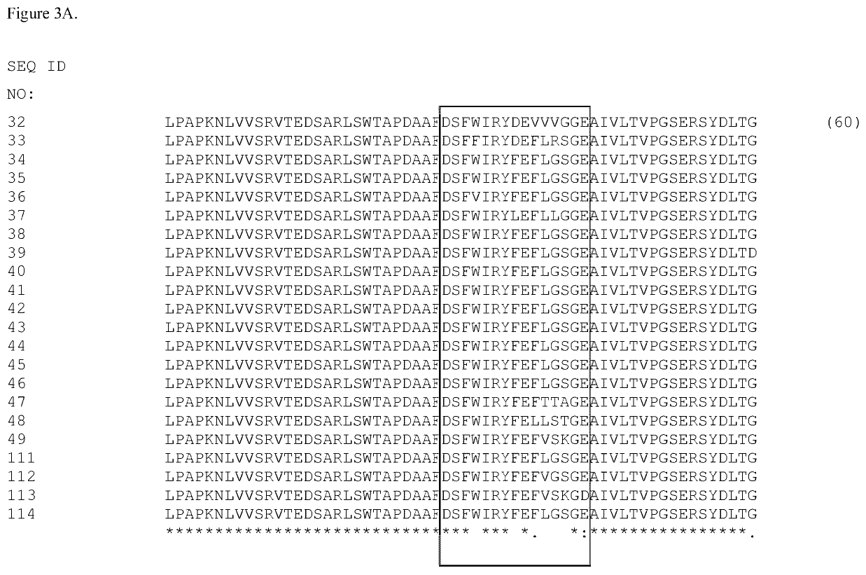

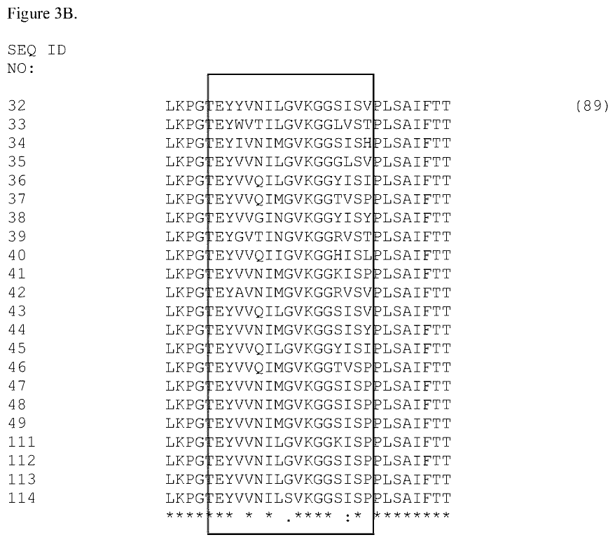

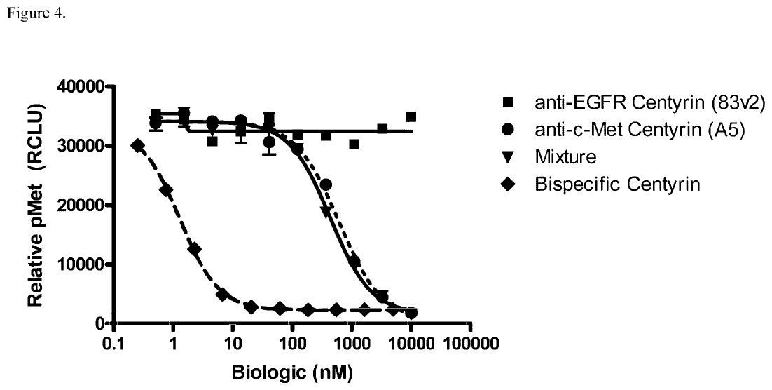

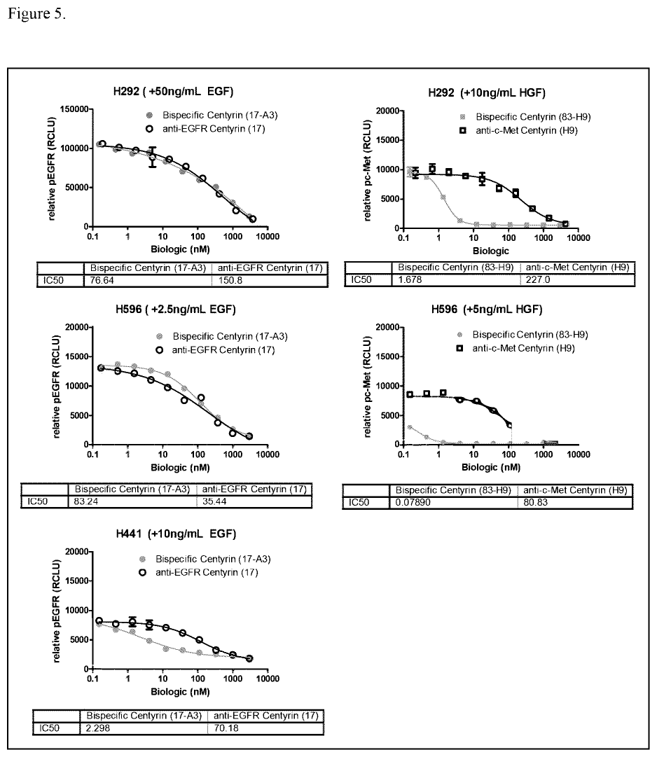

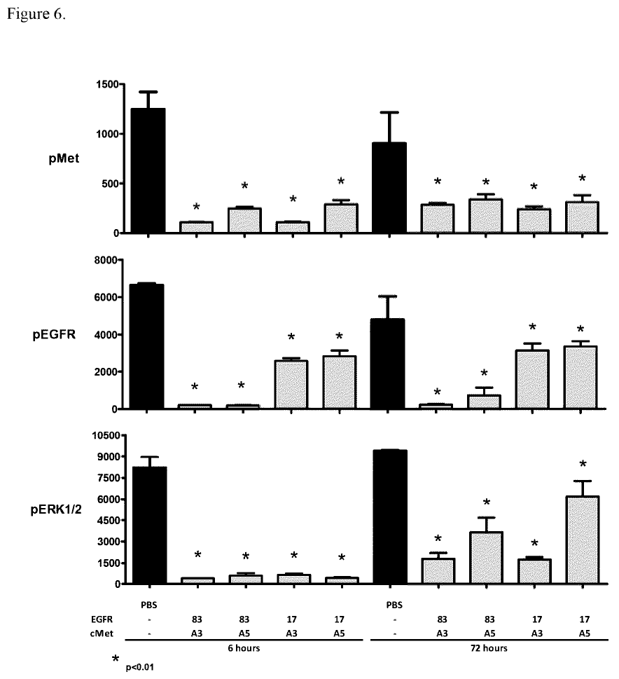

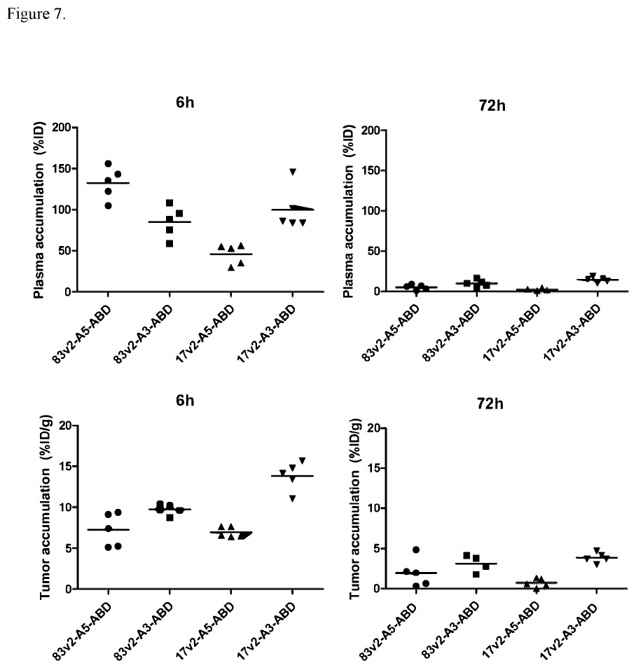

Library designed based on Tencon sequence may also have randomized alternative surfaces that form on a side of the FN3 domain and comprise two or more beta strands, and at least one loop. One such alternative surface is formed by amino acids in the C and the F beta-strands and the CD and the FG loops (a C-CD-F-FG surface). A library design based on Tencon alternative C-CD-F-FG surface and is shown in FIG. 1 and detailed generation of such libraries is described in U.S. Pat. Publ. No. US2013/0226834.

Library designed based on Tencon sequence also includes libraries designed based on Tencon variants, such as Tencon variants having substitutions at residues positions 11, 14, 17, 37, 46, 73, or 86 (residue numbering corresponding to SEQ ID NO: 1), and which variants display improved thermal stability. Exemplary Tencon variants are described in US Pat. Publ. No. 2011/0274623, and include Tencon27 (SEQ ID NO: 99) having substitutions E11R, L17A, N46V and E861 when compared to Tencon of SEQ ID NO: 1.

TABLE 1

| | Tencon |
|---|---|
| FN3 domain | (SEQ ID NO: 1) |
| A strand | 1-12 |
| AB loop | 13-16 |
| B strand | 17-21 |
| BC loop | 22-28 |
| C strand | 29-37 |
| CD loop | 38-43 |
| D strand | 44-50 |
| DE loop | 51-54 |
| E strand | 55-59 |
| EF loop | 60-64 |
| F strand | 65-74 |
| FG loop | 75-81 |
| G strand | 82-89 |

Tencon and other FN3 sequence based libraries can be randomized at chosen residue positions using a random or defined set of amino acids. For example, variants in the library having random substitutions can be generated using NNK codons, which encode all 20 naturally occurring amino acids. In other diversification schemes, DVK codons can be used to encode amino acids Ala, Trp, Tyr, Lys, Thr, Asn, Lys, Ser, Arg, Asp, Glu, Gly, and Cys. Alternatively, NNS codons can be used to give rise to all 20 amino acid residues and simultaneously reducing the frequency of stop codons. Libraries of FN3 domains with biased amino acid distribution at positions to be diversified can be synthesized for example using Slonomics® technology (http:_//www_sloning_com). This technology uses a library of pre-made double stranded triplets that act as universal building blocks sufficient for thousands of gene synthesis processes. The triplet library represents all possible sequence combinations necessary to build any desired DNA molecule. The codon designations are according to the well known IUB code.

The FN3 domains specifically binding EGFR or c-Met of the invention can be isolated by producing the FN3 library such as the Tencon library using cis display to ligate DNA fragments encoding the scaffold proteins to a DNA fragment encoding RepA to generate a pool of protein-DNA complexes formed after in vitro translation wherein each protein is stably associated with the DNA that encodes it (U.S. Pat. No. 7,842,476; Odegrip et al., Proc Nat Acad Sci USA 101, 2806-2810, 2004), and assaying the library for specific binding to EGFR and/or c-Met by any method known in the art and described in the Example. Exemplary well known methods which can be used are ELISA, sandwich immunoassays, and competitive and non-competitive assays (see, e.g., Ausubel et al., eds, 1994, Current Protocols in Molecular Biology, Vol. 1, John Wiley & Sons, Inc., New York). The identified FN3 domains specifically binding EGFR or c-Met are further characterized for their ability to block EGFR ligand such as EGF binding to EGFR, or HGF binding to c-Met, and for their ability to inhibit EGFR and/or c-Met signaling using methods described herein.

The FN3 domains specifically binding to EGFR or c-Met of the invention can be generated using any FN3 domain as a template to generate a library and screening the library for molecules specifically binding EGFR or c-Met using methods provided within. Exemplar FN3 domains that can be used are the 3rd FN3 domain of tenascin C (TN3) (SEQ ID NO: 75), Fibeon (SEQ ID NO: 76), and the 10^{th} FN3 domain of fibronectin (FN10)(SEQ ID NO: 77). Standard cloning and expression techniques are used to clone the libraries into a vector or synthesize double stranded cDNA cassettes of the library, to express, or to translate the libraries in vitro. For example ribosome display (Hanes and Pluckthun, Proc Natl Acad Sci USA, 94, 4937-4942, 1997), mRNA display (Roberts and Szostak, Proc Natl Acad Sci USA, 94, 12297-12302, 1997), or other cell-free systems (U.S. Pat. No. 5,643,768) can be used. The libraries of the FN3 domain variants may be expressed as fusion proteins displayed on the surface for example of any suitable bacteriophage. Methods for displaying fusion polypeptides on the surface of a bacteriophage are well known (U.S. Pat. Publ. No. 2011/0118144; Int. Pat. Publ. No. WO2009/085462; U.S. Pat. Nos. 6,969,108; 6,172,197; 5,223,409; 6,582,915; 6,472,147).

The FN3 domains specifically binding EGFR or c-Met of the invention can be modified to improve their properties such as improve thermal stability and reversibility of thermal folding and unfolding. Several methods have been applied to increase the apparent thermal stability of proteins and enzymes, including rational design based on comparison to highly similar thermostable sequences, design of stabilizing disulfide bridges, mutations to increase alpha-helix propensity, engineering of salt bridges, alteration of the surface charge of the protein, directed evolution, and composition of consensus sequences (Lehmann and Wyss, Curr Opin Biotechnol, 12, 371-375, 2001). High thermal stability may increase the yield of the expressed protein, improve solubility or activity, decrease immunogenicity, and minimize the need of a cold chain in manufacturing. Residues that can be substituted to improve thermal stability of Tencon (SEQ ID NO: 1) are residue positions 11, 14, 17, 37, 46, 73, or 86, and are described in US Pat. Publ. No. 2011/0274623. Substitutions corresponding to these residues can be incorporated to the FN3 domains or the bispecific FN3 domain containing molecules of the invention.

Another embodiment of the invention is an isolated FN3 domain that specifically binds EGFR and blocks binding of EGF to EGFR, comprising the sequence shown in SEQ ID NOs: 18-29, 107-110, 122-137, further comprising substitutions at one or more residue positions corresponding to positions 11, 14, 17, 37, 46, 73, and 86 in Tencon (SEQ ID NO: 1).

Another embodiment of the invention is an isolated FN3 domain that specifically binds c-Met and blocks binding of HGF to c-Met, comprising the sequence shown in SEQ ID NOs: 32-49 or 111-114, further comprising substitutions at one or more residue positions corresponding to positions 11, 14, 17, 37, 46, 73, and 86 in Tencon (SEQ ID NO: 1).

Exemplary substitutions are substitutions E11N, E14P, L17A, E37P, N46V, G73Y and E861 (numbering according to SEQ ID NO: 1).

In some embodiments, the FN3 domains of the invention comprise substitutions corresponding to substitutions L17A, N46V, and E861 in Tencon (SEQ ID NO: 1).

The FN3 domains specifically binding EGFR (FIG. 1) have an extended FG loop when compared to Tencon (SEQ ID NO: 1). Therefore, the residues corresponding to residues 11, 14, 17, 37, 46, 73, and 86 in Tencon (SEQ ID NO: 1) are residues 11, 14, 17, 37, 46, 73 and 91 in EGFR FN3 domains shown in FIGS. 1A and 1B except for the FN3 domain of SEQ ID NO: 24, wherein the corresponding residues are residues 11, 14, 17, 38, 74, and 92 due to an insertion of one amino acid in the BC Loop.

Another embodiment of the invention is an isolated FN3 domain that specifically binds EGFR and blocks binding of EGF to EGFR comprising the amino acid sequence shown in SEQ ID NOs: 18-29, 107-110, or 122-137, optionally having substitutions corresponding to substitutions L17A, N46V, and E861 in Tencon (SEQ ID NO: 1).

Another embodiment of the invention is an isolated FN3 domain that specifically binds c-Met and blocks binding of HGF to c-Met comprising the amino acid sequence shown in SEQ ID NOs: 32-49 or 111-114, optionally having substitutions corresponding to substitutions L17A, N46V, and E861 in Tencon (SEQ ID NO: 1).

Measurement of protein stability and protein lability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such as by gamma irradiation, chemical or heat dehydration, or any other action or force that may cause protein structure disruption. The stability of the molecule can be determined using standard methods. For example, the stability of a molecule can be determined by measuring the thermal melting (“TM”) temperature, the temperature in ° Celsius (° C.) at which half of the molecules become unfolded, using standard methods. Typically, the higher the TM, the more stable the molecule. In addition to heat, the chemical environment also changes the ability of the protein to maintain a particular three dimensional structure.

In one embodiment, the FN3 domains binding EGFR or c-Met of the invention exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same domain prior to engineering measured by the increase in the TM.

Chemical denaturation can likewise be measured by a variety of methods. Chemical denaturants include guanidinium hydrochloride, guanidinium thiocyanate, urea, acetone, organic solvents (DMF, benzene, acetonitrile), salts (ammonium sulfate, lithium bromide, lithium chloride, sodium bromide, calcium chloride, sodium chloride); reducing agents (e.g. dithiothreitol, beta-mercaptoethanol, dinitrothiobenzene, and hydrides, such as sodium borohydride), non-ionic and ionic detergents, acids (e.g. hydrochloric acid (HC1), acetic acid (CH₃COOH), halogenated acetic acids), hydrophobic molecules (e.g. phospholipids), and targeted denaturants. Quantitation of the extent of denaturation can rely on loss of a functional property, such as ability to bind a target molecule, or by physiochemical properties, such as tendency to aggregation, exposure of formerly solvent inaccessible residues, or disruption or formation of disulfide bonds.

In one embodiment, the FN3 domain of the invention binding EGFR or c-Met exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same scaffold prior to engineering, measured by using guanidinium hydrochloride as a chemical denaturant. Increased stability can be measured as a function of decreased tryptophan fluorescence upon treatment with increasing concentrations of guanidine hydrochloride using well known methods.

The FN3 domains of the invention may be generated as monomers, dimers, or multimers, for example, as a means to increase the valency and thus the avidity of target molecule binding, or to generate bi- or multispecific scaffolds simultaneously binding two or more different target molecules. The dimers and multimers may be generated by linking monospecific, bi- or multispecific protein scaffolds, for example, by the inclusion of an amino acid linker, for example a linker containing poly-glycine, glycine and serine, or alanine and proline. Exemplary linker include (GS)₂, (SEQ ID NO: 78), (GGGGS)₅ (SEQ ID NO: 79), (AP)₂ (SEQ ID NO: 80), (AP)₅ (SEQ ID NO: 81), (AP)₁₀, (SEQ ID NO: 82), (AP)₂₀ (SEQ ID NO: 83) and A(EAAAK)₅AAA (SEQ ID NO: 84), linkers. The dimers and multimers may be linked to each other in an N- to C-direction. The use of naturally occurring as well as artificial peptide linkers to connect polypeptides into novel linked fusion polypeptides is well known in the literature (Hallewell et al., J Biol Chem 264, 5260-5268, 1989; Alfhan et al., Protein Eng. 8, 725-731, 1995; Robinson & Sauer, Biochemistry 35, 109-116, 1996; U.S. Pat. No. 5,856,456).

Bispecific Agents Specifically Binding EGFR and c-Met

The bispecific agents that specifically bind EGFR and c-Met of the invention may provide a benefit in terms of specificity and reduced off-target toxicity when compared to small molecule EGFR and/or c-Met inhibitors. The present invention is based at least in part on the surprising finding that the bispecific agents specifically binding EGFR and c-Met provide a significantly improved synergistic inhibitory effect when compared to a mixture of EGFR-binding and c-Met-binding monospecific agents. The molecules may be tailored to specific affinity towards both EGFR and c-Met to maximize tumor penetration and retention. The bispecific agents that specifically bind EGFR and c-Met provide more efficient inhibition of EGFR and/or c-Met signaling pathways and inhibit tumor growth more efficiently than cetuximab (Erbitux®).

The bispecific agents specifically binding EGFR and c-Met may be formed by any polypeptide or a multimeric polypeptide that comprises an EGFR binding domain and a c-Met binding domain. The EGFR and the c-Met binding domains may be an antigen binding sites of an antibody, a VH/VL pair of an antibody, or another type of binding molecule such as a domain based on fibronectin type III (FN3) domain, a fibronectin type IX (FN9) domain, or any combination thereof.

The EGFR and c-Met binding polypeptides may be derived from existing monospecific EGFR and c-Met binding polypeptides or may be isolated de novo.

### Bispecific EGFR/c/Met FN3 Domain Containing Molecules

One embodiment of the invention is an isolated bispecific FN3 domain containing molecule comprising a first fibronectin type III (FN3) domain and a second FN3 domain, wherein the first FN3 domain specifically binds epidermal growth factor receptor (EGFR) and blocks binding of epidermal growth factor (EGF) to EGFR, and the second FN3 domain specifically binds hepatocyte growth factor receptor (c-Met) and blocks binding of hepatocyte growth factor (HGF) to c-Met.

The bispecific EGFR/c-Met FN3 domain containing molecules of the invention may be generated by covalently linking any EGFR-binding FN3 domain and any c-Met-binding FN3 domain of the invention directly or via a linker. Therefore, the first FN3 domain of the bispecific molecule may have characteristics as described above for the EGFR-binding FN3 domains, and the second FN3 domain of the bispecific molecule may have characteristics as described above for the c-Met-binding FN3 domains.

In one embodiment, the first FN3 domain of the bispecific EGFR/c-Met FN3 domain containing molecule inhibits EGF-induced EGFR phosphorylation at EGFR residue Tyrosine 1173 with an IC₅₀ value of less than about 2.5×10⁻⁶ M when measured in A431 cells using 50 ng/mL human EGF, and the second FN3 domain of the bispecific EGFR/c-Met FN3 domain containing molecule inhibits HGF-induced c-Met phosphorylation at c-Met residue Tyrosine 1349 with an IC₅₀ value of less than about 1.5×10⁻⁶ M when measured in NCI-H441 cells using 100 ng/mL human HGF.

In another embodiment, the first FN3 domain of the bispecific EGFR/c-Met FN3 domain containing molecule inhibits EGF-induced EGFR phosphorylation at EGFR residue Tyrosine 1173 with an IC₅₀ value of between about 1.8×10⁻⁸ M to about 2.5×10⁻⁶ M when measured in A431 cells using 50 ng/mL human EGF, and the second FN3 domain of the bispecific EGFR/c-Met FN3 domain containing molecule inhibits HGF-induced c-Met phosphorylation at c-Met residue Tyrosine 1349 with an ICs value between about 4×10⁻⁹ M to about 1.5×10⁻⁶ M when measured in NCI-H441 cells using 100 ng/mL human HGF.

In another embodiment, the first FN3 domain of the bispecific EGFR/c-Met FN3 domain containing molecule binds human EGFR with a dissociation constant (K_{D}) of less than about 1×10⁻⁸ M, and the second FN3 domain of the bispecific EGFR/c-Met FN3 domain containing molecule binds human c-Met with a K_{D} of less than about 5×10⁻⁸ M.

In the bispecific molecule binding both EGFR and c-Met, the first FN3 domain binds human EGFR with a K_{D} of between about 2×10⁻¹⁰ to about 1×10⁻⁸ M, and the second FN3 domain binds human c-Met with a K_{D} of between about 3×10⁻¹⁰ to about 5×10⁻⁸ M.

The affinity of the bispecific EGFR/c-Met molecule for EGFR and c-Met can be determined as described above for the monospecific molecules.

The first FN3 domain in the bispecific EGFR/c-Met molecule of the invention may block EGF binding to EGFR with an IC₅₀ value of between about 1×10⁻⁹ M to about 1.5×10⁻⁷ M in an assay employing A431 cells and detecting the amount of fluorescence from bound biotinylated EGF using streptavidin-phycoerythrin conjugate at 600 nM on A431 cells incubated with or without the first FN3 domain. The first FN3 domain in the bispecific EGFR/c-Met molecule of the invention may block EGF binding to the EGFR by at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% when compared to binding of EGF to EGFR in the absence of the first FN3 domains using the same assay conditions.

The second FN3 domain in the bispecific EGFR/c-Met molecule of the invention may block HGF binding to c-Met with an IC₅₀ value of between about 2×10⁻¹⁰ M to about 6×10⁻⁸ M in an assay detecting inhibition of binding of biotinylated HGF to c-Met-Fc fusion protein in the presence of the second FN3 domain. The second FN3 domain in the bispecific EGFR/c-Met molecule may block HGF binding to c-Met by at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% when compared to binding of HGF to c-Met in the absence of the second FN3 domain using the same assay conditions.

The bispecific EGFR/c-Met molecule of the invention may inhibit EGFR and/or c-Met signaling by at least 30%, 35%, 40%, 45%, 50%, 55%, 60%, 65%, 70%, 75%, 80%, 85%, 90%, 91%, 92%, 93%, 94%, 95/e, 96%, 97%, 98%, 99% or 100% when compared to the level of signaling in the absence of the bispecific EGFR/c-Met molecule of the invention using the same assay conditions.

EGFR and c-Met signaling may be measured using various well know methods as described above for the monospecific molecules.

The bispecific EGFR/c-Met molecules of the invention comprising the first FN3 domain specifically binding EGFR and the second FN3 domain specifically binding c-Met provide a significantly increased synergistic inhibition of EGFR and c-Met signaling and tumor cell proliferation when compared to the synergistic inhibition observed by a mixture of the first and the second FN3 domain. Synergistic inhibition can be assessed for example by measuring inhibition of ERK phosphorylation by the bispecific EGFR/c-Met FN3 domain containing molecules and by a mixture of two monospecific molecules, one binding EGFR and the other c-Met. The bispecific EGFR/c-Met molecules of the invention may inhibit ERK phosphorylation with an at least about 100 fold smaller, for example at least 500, 1000, 5000 or 10,000 fold smaller IC₅₀ value when compared to the IC₅₀ value for a mixture of two monospecific FN3 domains, indicating at least 100 fold increased potency for the bispecific EGFR/c-Met FN3 domain containing molecules when compared to the mixture of two monospecific FN3 domains. Exemplary bispecific EGFR-c-Met FN3 domain containing molecules may inhibit ERK phosphorylation with and IC₅₀ value of about 5×10⁻⁹ M or less. ERK phosphorylation may be measured using standard methods and methods described herein.

The bispecific EGFR/c-Met FN3 domain containing molecule of the invention may inhibit NCI-H292 cell proliferation with an ICs value that is at least 30-fold less when compared to the IC₅₀ value of inhibition of NCI-H292 cell growth with a mixture of the first FN3 domain and the second FN3, wherein the cell proliferation is induced with medium containing 10% FBS supplemented with 7.5 ng/mL HGF. The bispecific molecule of the invention may inhibit tumor cell proliferation with an IC₅₀ value that is about 30, 40, 50, 60, 70, 80, 90, 100, 150, 200, 300, 400, 500, 600, 700, 800, or about 1000 fold less when compared to the IC₅₀ value of inhibition of tumor cell proliferation with a mixture of the first FN3 domain and the second FN3 domain. Inhibition of tumor cell proliferation may be measured using standard methods and methods described herein.

Another embodiment of the invention is a bispecific FN3 domain containing molecule comprising a first fibronectin type III (FN3) domain and a second FN3 domain, wherein the first FN3 domain specifically binds epidermal growth factor receptor (EGFR) and blocks binding of epidermal growth factor (EGF) to EGFR, and the second FN3 domain specifically binds hepatocyte growth factor receptor (c-Met), and blocks binding of hepatocyte growth factor (HGF) to c-Met, wherein
the first FN3 domain comprises
   an FG loop comprising the sequence HNVYKDTNX₉RGL (SEQ ID NO: 179) or the sequence LGSYVFEHDVML (SEQ ID NO: 180), wherein X₉ is M or I; and
   a BC loop comprising the sequence X₁X₂X₃X₄X₅X₆X₇X₈ (SEQ ID NO: 181),
   wherein
      X₁ is A, T, G or D;
      X₂ is A, D, Y or W;
      X₃ is P, D or N;
      X₄ is L or absent;
      X₅ is D, H, R, G, Y or W;
      X₆ is G, D or A;
      X₇ is A, F, G, H or D; and
      X₈ is Y, F or L; and
the second FN3 domain comprises
   a C strand and a CD loop comprising the sequence DSFX₁₀IRYX₁₁E X₁₂X₁₃X₁₄X₁₅GX₁₆ (SEQ ID NO: 184), wherein
      X₁₀ is W, F or V;
      X₁₁ is D, F or L;
      X₁₂ is V, F or L;
      X₁₃ is V, L or T;
      X₁₄ is V, R, G, L, T or S;
      X₁₅ is G, S, A, T or K; and
      X₁₆ is E or D; and
   a F strand and a FG loop comprising the sequence TEYX₁₇VX₁₈X₁₉X₂₀V KGGX₂₁X₂₂SX₂₃ (SEQ ID NO: 185), wherein
      X₁₇ is Y, W, I, V, G or A;
      X₁₈ is N, T, Q or G;
      X₁₉ is L, M, N or I;
      X₂₀ is G or S;
      X₂₁ is S, L, G, Y, T, R, H or K;
      X₂₂ is I, V or L; and
      X₂₃ is V, T, H, I, P, Y or L.

In another embodiment, the bispecific molecule comprises the first FN3 domain that binds EGFR comprising the sequence:

(SEQ ID NO: 182)
LPAPKNLVVSEVTEDSLRLSWX₁X₂X₃X₄X₅X₆X₇X₈DSFLIQYQESEKV
GEAINLTVPGSERSYDLTGLKPGTEYTVSIYGVHNVYKDTNX₉RGL PL
SAEFTT,

or the sequence

(SEQ ID NO: 183)
LPAPKNLVVSEVTEDSLRLSWX₁X₂X₃X₄X₅X₆X₇X₈DSFLIQYQESEKV
GEAINLTVPGSERSYDLTGLKPGTEYTVSIYGV LGSYVFEHDVMLPLSA
EFTT,

wherein in the SEQ ID NOs: 182 and 183;
X₁ is A, T, G or D;
X₂ is A, D, Y or W;
X₃ is P, D or N;
X₄ is L or absent;
X₅ is D, H, R, G, Y or W;
X₆ is G, D or A;
X₇ is A, F, G, H or D;
X₈ is Y, F or L; and
X₉ is M or I.

In another embodiment, the bispecific molecule comprises the second FN3 domain that binds c-Met comprising the sequence

(SEQ ID NO: 186)
LPAPKNLVVSRVTEDSARLSWTAPDAAF DSFX₁₀IRYX₁₁EX₁₂X₁₃
X₁₄X₁₅GX₁₆AIVLTVPGSERSYDLTGLKPG TEYX₁₇VX₁₈IX₁₉X₂₀
VKGGX₂₁X₂₂SX₂₃PLSAEFTT,

wherein
X₁₀ is W, F or V; and
X₁₁ is D, F or L;
X₁₂ is V, F or L;
X₁₃ is V, L or T;
X₁₄ is V, R, G, L, T or S;
X₁₅ is G, S, A, T or K;
X₁₆ is E or D;
X₁₇ is Y, W, I, V, G or A;
X₁₈ is N, T, Q or G;
X₁₄ is L, M, N or I;
X₂₀ is G or S;
X₂₁ is S, L, G, Y, T, R, H or K;
X₂₂ is 1, V or L; and
X₂₃ is V, T, H, I, P, Y or L.

Exemplary bispecific EGFR/c-Met FN3 domain containing molecules comprise the amino acid sequence shown in SEQ ID NOs: 50-72, 106, 118-121, or 138.167.

The bispecific EGFR/c-Met molecules of the invention comprise certain structural characteristics associated with their functional characteristics, such as inhibition of EGFR autophosphorylation, such as the FG loop of the first FN3 domain that binds EGFR comprising the sequence HNVYKDTNX₉RGL (SEQ ID NO: 179) or the sequence LGSYVFEHDVML (SEQ ID NO: 180), wherein X₉ is M or I.

In one embodiment, the bispecific EGFR/c-Met FN3 domain containing molecules of the invention
inhibit EGF-induced EGFR phosphorylation at EGFR residues Tyrosine 1173 with an IC₅₀ value of less than about 8×10⁻⁷ M when measured in H292 cells using 50 ng/mL human EGF;
   inhibit HGF-induced c-Met phosphorylation at c-Met residue Tyrosine 1349 with an IC₅₀ value of less than about 8.4×10⁻⁷ M when measured in NCI-H441 cells using 100 ng/mL human HGF;
inhibit HGF-induced NCI-H292 cell proliferation with an IC₅₀ value of less than about 9.5×10⁻⁶M wherein the cell proliferation is induced with 10% FBS containing 7.5 ng HGF;
bind EGFR with a K_{D} of less than about 2.0×10⁻⁸ M; or
bind c-Met with a K_{D} of less than about 2.0×10⁻⁸ M.

In another embodiment, the bispecific EGFR/c-Met FN3 domain containing molecules of the invention
inhibit EGF-induced EGFR phosphorylation at EGFR residues Tyrosine 1173 with and IC₅₀ of between about 4.2×10⁻⁹ M and 8×10⁻⁷ M when measured in H292 cells using 50 ng/mL human EGF;
inhibit HGF-induced c-Met phosphorylation at c-Met residues Tyrosine 1349 with an IC₅₀ value of between about 2.4×10⁻⁸ M to about 8.4×10⁻⁷ M when measured in NCI-H441 cells using 100 ng/mL human HGF;
inhibit HGF-induced NCI-H292 cell proliferation with an IC₅₀ value between about 2.3×10⁻⁸ M to about 9.5×10⁻⁶M wherein the cell proliferation is induced with 10% FBS containing 7.5 ng HGF;
bind EGFR with a Ku of between about 2×10⁻¹⁰ M to about 2.0×10⁻⁸ M; or
bind c-Met with a K of between about 1×10⁻⁹ M to about 2.0×10⁻⁸ M.

In one embodiment, the bispecific EGFR/c-Met molecules comprise the EGFR-binding FN3 domain comprising the sequence

(SEQ ID NO: 182)
LPAPKNLVVSEVTEDSLRLSWX₁X₂X₃X₄X₅X₆X₇X₈DSFLIQYQESEKV
GEAINLTVPGSERSYDLTGLKPGTEYTVSIYGV HNVYKDTNX₉RGL PL
SAEFTT,

wherein
X₁ is D;
X₂ is D;
X₃ is P;
X₄ is absent;
X₅ is H or W;
X₆ is A;
X₇ is F
X₈ is Y; and
X₉ is M or I; and
the c-Met-binding FN3 domain comprising the sequence

(SEQ ID NO: 186)
LPAPKNLVVSRVTEDSARLSWTAPDAAF DSFX₁₀IRYX₁₁EX₁₂X₁₃
X₁₄X₁₅GX₁₆AIVLTVPGSERSYDLTGLKPG TEYX₁₇VX₁₈IX₁₉X₂₀V
KGGX₂₁X₂₂SX₂₃ PLSAEFTT,

wherein
X₁₀ is W;
X₁₁ is F;
X₁₂ is F;
X₁₃ is V or L;
X₁₄ is G or S;
X₁₅ is S or K;
X₁₆ is E or D;
X₁₇ is V;
X₁₈ is N;
X₁₉ is L or M;
X₂₀ is G or S;
X₂₁ is S or K;
X₂₂ is I; and
X₂₃ is P.

Exemplary bispecific EGFR/c-Met molecules are those having the sequence shown in SEQ ID NOs: 57, 61, 62, 63, 64, 65, 66, 67 and 68.

The bispecific molecules of the invention may further comprise substitutions at one or more residue positions in the first FN3 domain and/or the second FN3 domain corresponding to positions 11, 14, 17, 37, 46, 73 and 86 in Tencon (SEQ ID NO: 1) as described above, and a substitution at position 29. Exemplary substitutions are substitutions E11N, E14P, L17A, E37P, N46V, G73Y, E861 and D29E (numbering according to SEQ ID NO: 1). Skilled in the art will appreciate that other amino acids can be used for substitutions, such as amino acids within a family of amino acids that are related in their side chains as described infra. The generated variants can be tested for their stability and binding to EGFR and/or c-Met using methods herein.

In one embodiment, the bispecific EGFR/c-Met FN3 domain containing molecule comprises the first FN3 domain that binds specifically EGFR and the second FN3 domain that binds specifically c-Met, wherein the first FN3 domain comprises the sequence:

(SEQ ID NO: 187)
LPAPKNLVVSX₂₄VTX₂₅DSX₂₆RLSWDDPX₂₇AFYX₂₈SFLIQYQX₂₉S
EKVGEAIX₃₀LTVPGSERSYDLTGLKPGTEYTVSIYX₃₁VHNVYKDTN
X₃₂RGLPLSAX₃₃FTT,

wherein
X₂₄ is E, N or R;
X₂₅ is E or P;
X₂₆ is L or A;
X₂₇ is H or W;
X₂₈ is E or D;
X₂₉ is E or P;
X₃₀ is N or V;
X₃₁ is G or Y;
X₃₂ is M or I; and
X₃₃ is E or I;
   and the second FN3 domain comprises the sequence:

(SEQ ID NO: 188)
LPAPKNLVVSX₃₄VTX₃₅DSX₃₆RLSWTAPDAAFDSFWIRYFX₃₇FX₃₈
X₃₉X₄₀GX₄₁AIX₄₂LTVPGSERSYDLTGLKPGTEYVVNIX₄₃X₄₄VKG
GX₄₅ISPPLSAX₄₆FTT;

wherein
X₃₄ is E, N or R;
X₃₅ is E or P;
X₃₆ is L or A;
X₃₇ is E or P;
X₃₈ is V or L;
X₃₉ is G or S;
X₄₀ is S or K;
X₄₁ is E or D;
X₄₂ is N or V;
X₄₃ is L or M;
X₄₄ is G or S;
X₄₅ is S or K; and
X₄₆ is E or I.

In other embodiments, the bispecific EGFR/c-Met FN3 domain containing molecule comprises the first FN3 domain comprising an amino acid sequence at least 87%, 88%, 89%, 90%, 91%, 92%, 93%, 94%, 95%, 96%, 97%, 98% or 99% identical to the amino acid sequence of SEQ ID NO: 27, and the second FN3 domain comprising an amino acid sequence at least 83%, 84%, 85%, 86%, 87%, 88%, 89%, 90%, 91%, 92%, 93%, 94%, 95%, 96%, 97%, 98% or 99% identical to the amino acid sequence of SEQ ID NO: 41.

The bispecific EGFR/c-Met FN3 domain containing molecules of the invention may be tailored to a specific affinity towards EGFR and c-Met to maximize tumor accumulation.

Another embodiment of the invention is an isolated bispecific FN3 domain containing molecule comprising a first fibronectin type III (FN3) domain and a second FN3 domain, wherein the first FN3 domain specifically binds epidermal growth factor receptor (EGFR) and blocks binding of epidermal growth factor (EGF) to EGFR, and the second FN3 domain specifically binds hepatocyte growth factor receptor (c-Met), and blocks binding of hepatocyte growth factor (HGF) to c-Met, wherein the first FN3 domain and the second FN3 domain is isolated from a library designed based on Tencon sequence of SEQ ID NO: 1.

The bispecific EGFR/c-Met FN3 domain containing molecule of the invention can be generated by covalently coupling the EGFR-binding FN3 domain and the c-Met binding FN3 domain of the invention using well known methods. The FN3 domains may be linked via a linker, for example a linker containing poly-glycine, glycine and serine, or alanine and proline. Exemplary linker include (GS)₂, (SEQ ID NO: 78), (GGGGS)₅ (SEQ ID NO: 79), (AP)₂ (SEQ ID NO: 80), (AP)₅ (SEQ ID NO: 81), (AP)₁₀ (SEQ ID NO: 82), (AP)₂₀ (SEQ ID NO: 83), A(EAAAK)₅AAA (SEQ ID NO: 84), linkers. The use of naturally occurring as well as artificial peptide linkers to connect polypeptides into novel linked fusion polypeptides is well known in the literature (Hallewell et al., J Biol Chem 264, 5260-5268, 1989; Alfthan et al., Protein Eng. 8, 725-731, 1995; Robinson & Sauer, Biochemistry 35, 109-116, 1996; U.S. Pat. No. 5,856,456). The bispecific EGFR/c-Met molecules of the invention may be linked together from a C-terminus of the first FN3 domain to the N-terminus of the second FN3 domain, or from the C-terminus of the second FN3 domain to the N-terminus of the first FN3 domain. Any EGFR-binding FN3 domain may be covalently linked to a c-Met-binding FN3 domain. Exemplary EGFR-binding FN3 domains are domains having the amino acid sequence shown in SEQ ID NOs: 18-29, 107-110, and 122-137, and exemplary c-Met binding FN3 domains are domains having the amino acid sequence shown in SEQ ID NOs: 32-49 and 111-114. The EGFR-binding FN3 domains to be coupled to a bispecific molecule may additionally comprise an initiator methionine (Met) at their N-terminus.

Variants of the bispecific EGFR/c-Met FN3 domain containing molecules are within the scope of the invention. For example, substitutions can be made in the bispecific EGFR/c-Met FN3 domain containing molecule as long as the resulting variant retains similar selectivity and potency towards EGFR and c-Met when compared to the parent molecule. Exemplary modifications are for example conservative substitutions that will result in variants with similar characteristics to those of the parent molecules. Conservative substitutions are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981). Non-conservative substitutions can be made to the bispecific EGFR/c-Met FN3 domain containing molecule that involves substitutions of amino acid residues between different classes of amino acids to improve properties of the bispecific molecules. Whether a change in the amino acid sequence of a polypeptide or fragment thereof results in a functional homolog can be readily determined by assessing the ability of the modified polypeptide or fragment to produce a response in a fashion similar to the unmodified polypeptide or fragment using the assays described herein. Peptides, polypeptides or proteins in which more than one replacement has taken place can readily be tested in the same manner.

The bispecific EGFR/c-Met FN3 domain containing molecules of the invention may be generated as dimers or multimers, for example, as a means to increase the valency and thus the avidity of target molecule binding. The multimers may be generated by linking one or more EGFR-binding FN3 domain and one or more c-Met-binding FN3 domain to form molecules comprising at least three individual FN3 domains that are at least bispecific for either EGFR or c-Met, for example by the inclusion of an amino acid linker using well known methods.

Another embodiment of the invention is a bispecific FN3 domain containing molecule comprising a first fibronectin type III (FN3) domain and a second FN3 domain, wherein the first FN3 domain specifically binds epidermal growth factor receptor (EGFR) and blocks binding of epidermal growth factor (EGF) to EGFR, and the second FN3 domain specifically binds hepatocyte growth factor receptor (c-Met), and blocks binding of hepatocyte growth factor (HGF) to c-Met comprising the amino acid sequence shown in SEQ ID NOs: 50-72, 106 or 138-165.

### Half-Life Extending Moieties

The bispecific EGFR/c-Met FN3 domain containing molecules or the monospecific EGFR or c-Met binding FN3 domains of the invention may incorporate other subunits for example via covalent interaction. In one aspect of the invention, the bispecific EGFR/c-Met FN3 domain containing molecules of the invention further comprise a half-life extending moiety. Exemplary half-life extending moieties are albumin, albumin variants, albumin-binding proteins and/or domains, transferrin and fragments and analogues thereof, and Fc regions. An exemplary albumin-binding domain is shown in SEQ ID NO: 117.

All or a portion of an antibody constant region may be attached to the molecules of the invention to impart antibody-like properties, especially those properties associated with the Fc region, such as Fc effector functions such as C1q binding, complement dependent cytotoxicity (CDC), Fc receptor binding, antibody-dependent cell-mediated cytotoxicity (ADCC), phagocytosis, down regulation of cell surface receptors (e.g., B cell receptor, BCR), and can be further modified by modifying residues in the Fc responsible for these activities (for review; see Strohl, Curr Opin Biotechnol. 20, 685-691.2009).

Additional moieties may be incorporated into the bispecific molecules of the invention such as polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein scaffold coding sequences and may be generated by standard cloning and expression techniques. Alternatively, well known chemical coupling methods may be used to attach the moieties to recombinantly produced molecules of the invention.

A pegyl moiety may for example be added to the bispecific or monospecific molecules of the invention by incorporating a cysteine residue to the C-terminus of the molecule and attaching a pegyl group to the cysteine using well known methods. Exemplary bispecific molecules with the C-terminal cysteine are those having the amino acid sequence shown in SEQ IN NO: 170-178.

Monospecific and bispecific molecules of the invention incorporating additional moieties may be compared for functionality by several well known assays. For example, altered properties of monospecific and/or bispecific molecules due to incorporation of Fc domains and/or Fc domain variants may be assayed in Fc receptor binding assays using soluble forms of the receptors, such as the FcγRI, FcγRII, FcγRIII or FcRn receptors, or using well known cell-based assays measuring for example ADCC or CDC, or evaluating pharmacokinetic properties of the molecules of the invention in in vivo models.

### Polynucleotides, Vectors, Host Cells

The invention provides for nucleic acids encoding the EGFR-binding or c-Met binding FN3 domains or the bispecific EGFR/c-Met FN3 domain containing molecules of the invention as isolated polynucleotides or as portions of expression vectors or as portions of linear DNA sequences, including linear DNA sequences used for in vitro transcription/translation, vectors compatible with prokaryotic, eukaryotic or filamentous phage expression, secretion and/or display of the compositions or directed mutagens thereof. Certain exemplary polynucleotides are disclosed herein, however, other polynucleotides which, given the degeneracy of the genetic code or codon preferences in a given expression system, encode the EGFR-binding or c-Met binding FN3 domains or the bispecific EGFR/c-Met FN3 domain containing molecules of the invention are also within the scope of the invention.

One embodiment of the invention is an isolated polynucleotide encoding the FN3 domain specifically binding EGFR having the amino acid sequence of SEQ ID NOs: 18-29, 107-110, or 122-137.

One embodiment of the invention is an isolated polynucleotide comprising the polynucleotide sequence of SEQ ID NOs: 97-98 or 168-169.

One embodiment of the invention is an isolated polynucleotide encoding the FN3 domain specifically binding c-Met having the amino acid sequence of the sequence shown in SEQ ID NOs: 32-49 or 111-114.

One embodiment of the invention is an isolated polynucleotide encoding the bispecific EGFR/-c-Met FN3 domain containing molecule having the amino acid sequence of SEQ ID NOs: 50-72, 106, 118-121 or 138-165.

One embodiment of the invention is an isolated polynucleotide comprising the polynucleotide sequence of SEQ ID NOs: 115-116 or 166-167.

The polynucleotides of the invention may be produced by chemical synthesis such as solid phase polynucleotide synthesis on an automated polynucleotide synthesizer and assembled into complete single or double stranded molecules. Alternatively, the polynucleotides of the invention may be produced by other techniques such as PCR followed by routine cloning. Techniques for producing or obtaining polynucleotides of a given known sequence are well known in the art.

The polynucleotides of the invention may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

Another embodiment of the invention is a vector comprising at least one polynucleotide of the invention. Such vectors may be plasmid vectors, viral vectors, vectors for baculovirus expression, transposon based vectors or any other vector suitable for introduction of the polynucleotides of the invention into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

Another embodiment of the invention is a host cell comprising the vector of the invention. A monospecific EGFR-binding or c-Met binding FN3 domain or the bispecific EGFR/c-Met FN3 domain containing molecule of the invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, NY (1989); Harlow and Lane, Antibodies, a Laboratory Manual, Cold Spring Harbor, NY (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

The host cell chosen for expression may be of mammalian origin or may be selected from COS-1, COS-7, HEK293, BHK21, CHO, BSC-1, He G2, SP2/0. HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21(DE3), BL21-GOLD(DE3), XLI-Blue, JM109, HMS174, HMS174(DE3), and any of the natural or engineered E. coli spp, Klebsiella spp., or Pseudomonas spp strains.

Another embodiment of the invention is a method of producing the isolated FN3 domain specifically binding EGFR or c-Met of the invention or the isolated bispecific EGFR/c-Met FN3 domain containing molecule of the invention, comprising culturing the isolated host cell of the invention under conditions such that the isolated FN3 domain specifically binding EGFR or c-Met or the isolated bispecific EGFR/c-Met FN3 domain containing molecule is expressed, and purifying the domain or molecule.

The FN3 domain specifically binding EGFR or c-Met or the isolated bispecific EGFR/c-Met FN3 domain containing molecule of the invention can be purified from recombinant cell cultures by well-known methods, for example by protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or high performance liquid chromatography (HPLC).

### Bispecific EGFR/c-Met Antibodies

The bispecific EGFR/c-Met antibodies may be generated de novo or may be engineered from existing monospecific anti-EGFR and anti-c-Met antibodies.

Exemplary anti-EGFR antibodies that may be used to engineer bispecific molecules are for example panitumumab (ABX-EGF), nimotuzumab, necitumumab, matuzumab, and those described for example in: U.S. Pat. Nos. 7,595,378, 7,247,301, U.S. Pat. Publ. No. US2011/0256142, U.S. Pat. No. 5,891,996, 5,212,290, 5,558,864, or 7,589,180. For example, antibody VH domain having the amino acid sequence shown in SEQ ID NO: 189 or 191 and antibody VL domain having the amino acid sequences shown in SEQ ID NO: 190 or 192 may be used.

Exemplary anti-c-Met antibodies that may be used to engineer bispecific molecules are for example Rilotumumab, Onartuzumab, Ficlatuzumab, and those described for example in PCT Intl. Publ. No. WO2011/110642, US Pat. Publ. No. US2004/0166544, PCT Intl. Publ. No. WO2005/016382, or PCT Intl. Publ. No. WO2006/015371. For example, antibody VH domain having the amino acid sequence shown in SEQ ID NO: 193 or 195 and antibody VL domain having the amino acid sequences shown in SEQ ID NO: 194 or 196 may be used. The heavy and light chain amino acid sequences of the antibodies identified by their United States Adopted Names (USAN) is available via the American Medical Association at http://_www_ama-assn_org or via the CAS registry.

Monospecific EGFR and c-Met biding variable domains may be selected de novo from for example a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-314, 1996: Sheets et al., PITAS (USA) 95:6157-6162, 1998; Hoogenboom and Winter, J Mol Biol 227:381, 1991; Marks et al., J Mol Biol 222:581, 1991), and subsequently engineered into a bispecific format. The monospecific EGFR and c-Met binding variable domains may be isolated for example from phage display libraries expressing antibody heavy and light chain variable regions as fusion proteins with bacteriophage pIX coat protein as described in Shi et al (2010) J. Mol. Biol. 397:385-96 and PCT Intl. Publ. No. WO09/085462). The antibody libraries are screened for binding to human EGFR or c-Met extracellular domains and the obtained positive clones are further characterized and the Fabs isolated from the clone lysates. Such phage display methods for isolating human antibodies are established in the art. See for example: U.S. Pat. Nos. 5,223,409; 5,403,484; and 5,571,698, 5,427,908, 5,580,717, 5,969,108, 6,172,197, 5,885,793; 6,521,404; 6,544,731; 6,555,313; 6,582,915 and 6,593,081. The obtained de novo variable regions binding EGFR or c-Met are engineered to bispecific formats using the methods described herein.

### Bispecific Antibody Formats

Antibodies of the present invention have two or more antigen binding sites and are bispecific. Bispecific antibodies of the invention include antibodies having a full length antibody structure.

“Full length antibody” as used herein refers to an antibody having two full length antibody heavy chains and two full length antibody light chains. A full length antibody heavy chain (HC) consists of well known heavy chain variable and constant domains VH, CH1, CH2, and CH3. A full length antibody light chain (LC) consists of well known light chain variable and constant domains VL and CL. The full length antibody may be lacking the C-terminal lysine (K) in either one or both heavy chains.

The term “Fab-arm” or “half molecule” refers to one heavy chain-light chain pair that specifically binds an antigen.

Full length bispecific antibodies of the invention may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e. an epitope on EGFR and an epitope on c-Met.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351Y_F405A_Y407V, T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S.

Pat. Publ. No. US2012/0149876 or U.S. Pat. Publ. No. US2013/0195849 In addition to methods described above, bispecific antibodies of the invention may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Int. Pat. Publ. No. WO2011/131746. In the methods, the first monospecific bivalent antibody (e.g., anti-c-Met antibody) and the second monospecific bivalent antibody (e.g., anti-EGFR antibody) are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

### Bispecific EGFR/c-Met Antibodies

The bispecific EGFR/c-Met antibodies of the invention may provide a benefit in terms of specificity and reduced off-target toxicity when compared to small molecule EGFR and/or c-Met inhibitors. The present invention is based at least in part on the surprising finding that the bispecific EGFR/c-Met antibodies of the invention provide a significantly improved synergistic inhibitory effect when compared to a mixture of EGFR-binding and c-Met-binding monospecific antibodies or published bispecific EGFR/c-Met antibodies. Depending on the assay, the synergistic effect observed varied between about 14- to over about 800-fold. The bispecific EGFR/c-Met antibodies of the invention provide more efficient inhibition of EGFR and c-Met signaling pathways and inhibit tumor growth more efficiently than cetuximab (Erbitux®). The bispecific EGFR/c-Met antibodies of the invention inhibit EGFR signaling in tumors and/or tumor cell lines having EGFR activating mutations and/or mutations in EGFR that are known to result in resistance to treatments with tyrosine kinase inhibitors such as gefitinib, and inhibit c-Met signaling pathway, a pathway identified to be upregulated and to provide a compensatory signaling upon treatment with EGFR tyrosine kinase inhibitors in cancers such as NSCLC. The bispecific EGFR/c-Met antibodies of the invention, in addition to directly inhibiting EGFR and c-Met signaling, display antitumor activity through enhanced antibody dependent cell cytotoxicity (ADCC) and degradation of the EGFR and c-Met receptors. Contrary to the current EGFR therapies (cetuximab and panitumumab), the bispecific EGFR/c-Met antibodies of the invention induce, via enhanced ADCC, killing of tumor cells having KRAS mutations.

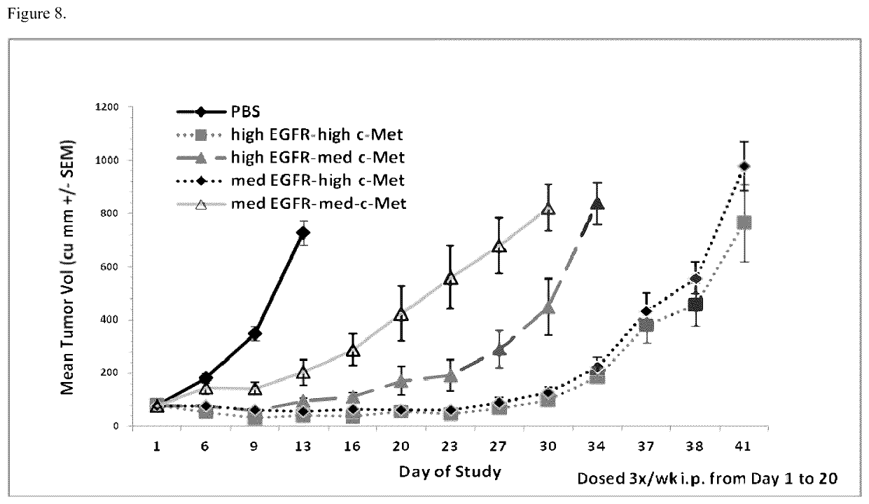

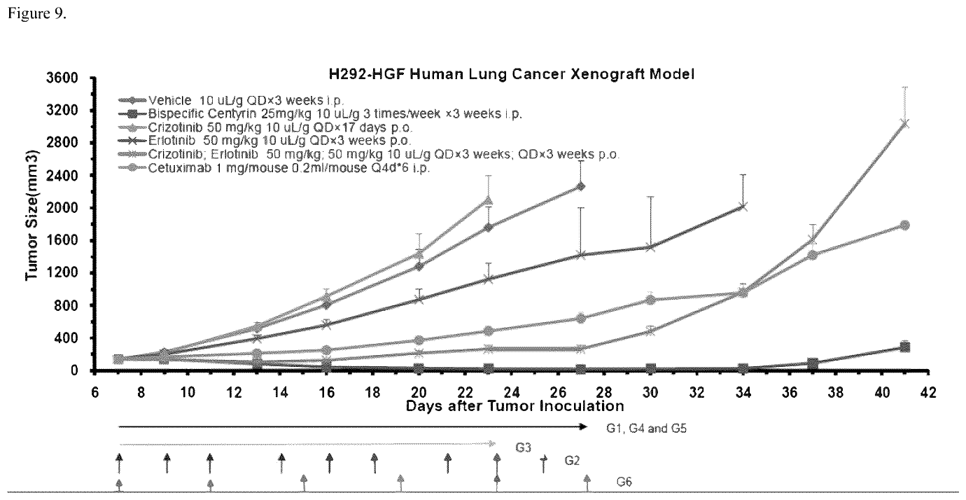

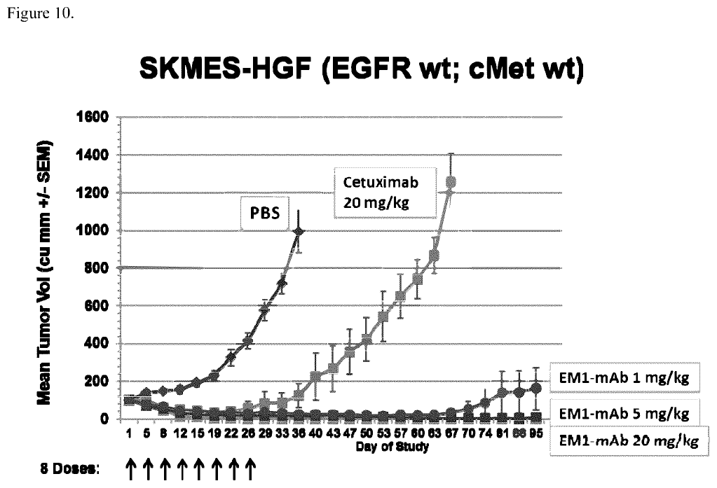

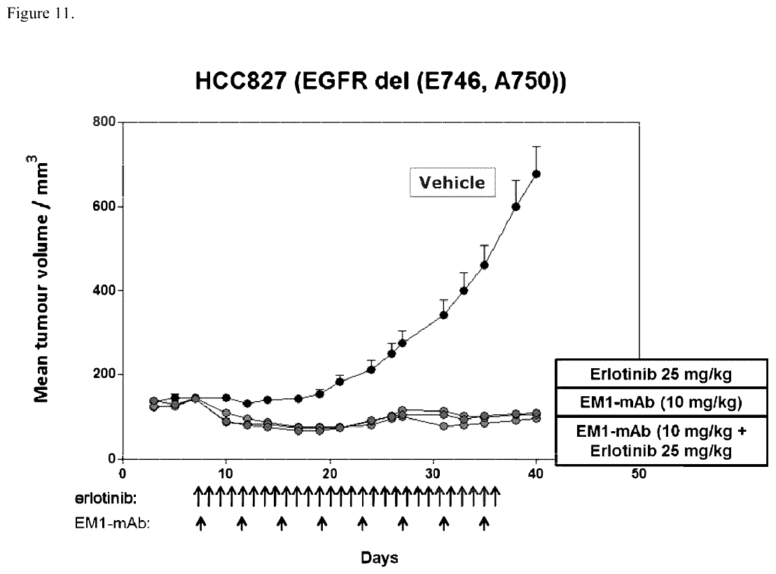

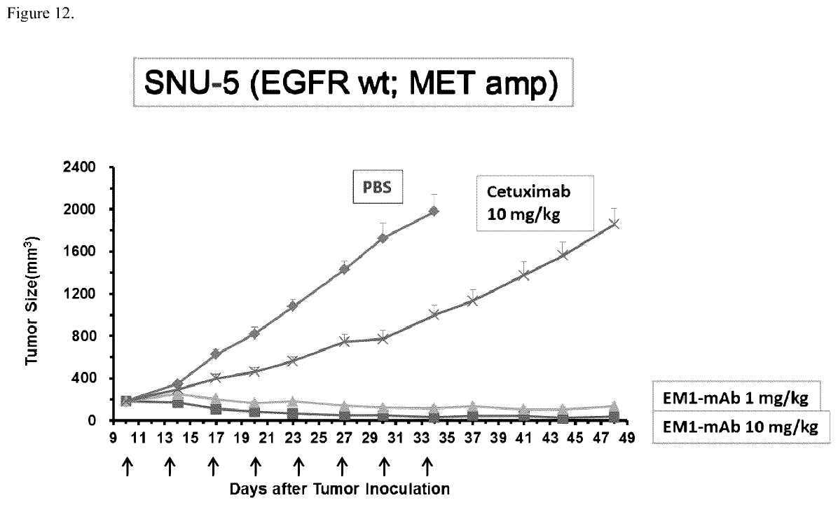

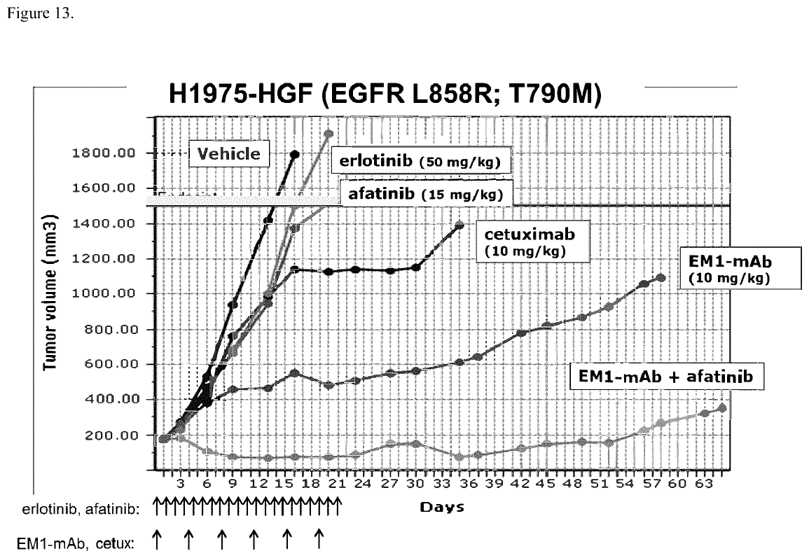

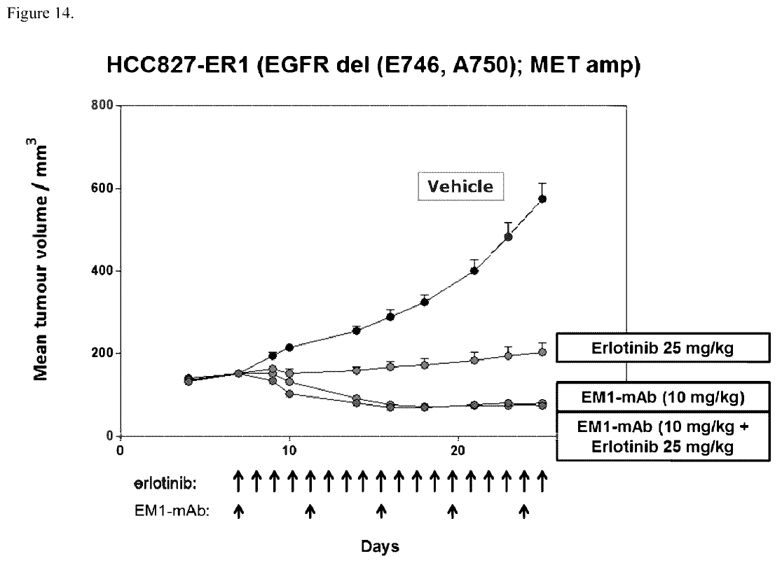

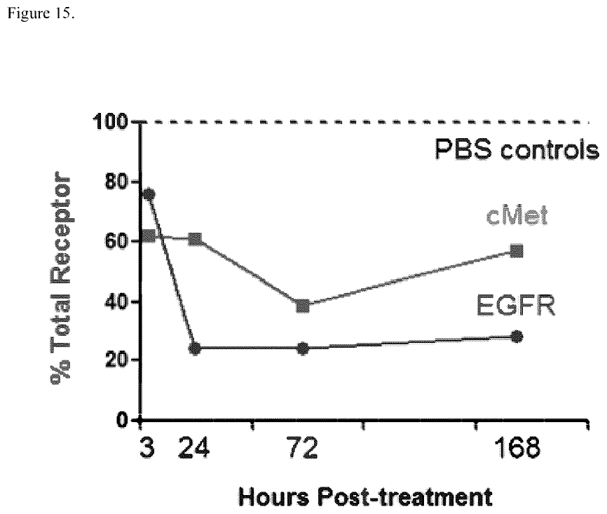

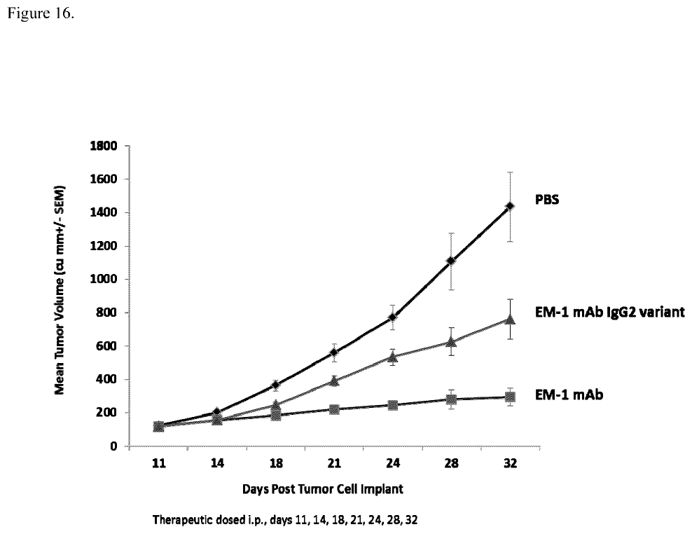

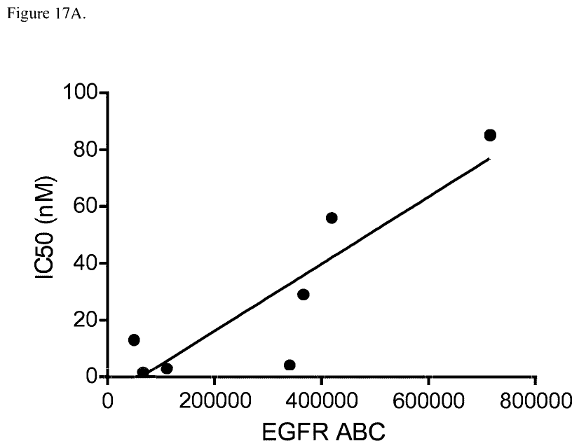

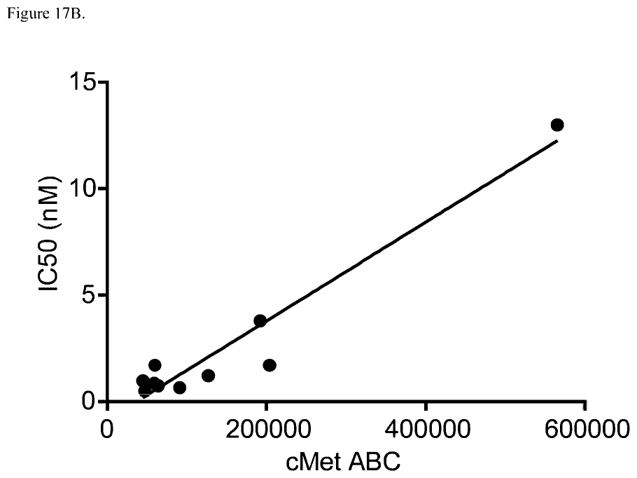

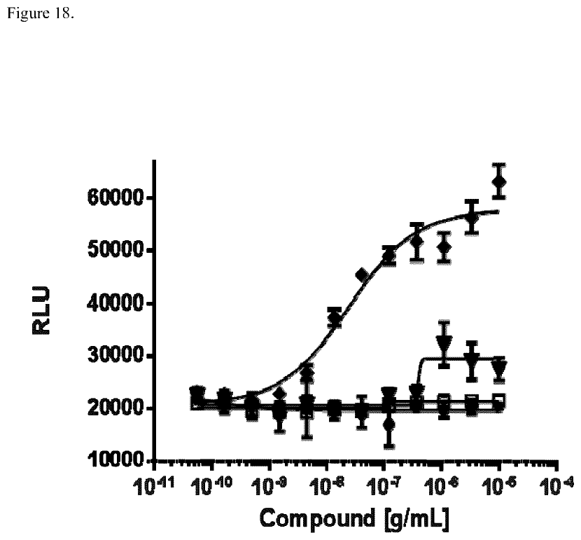

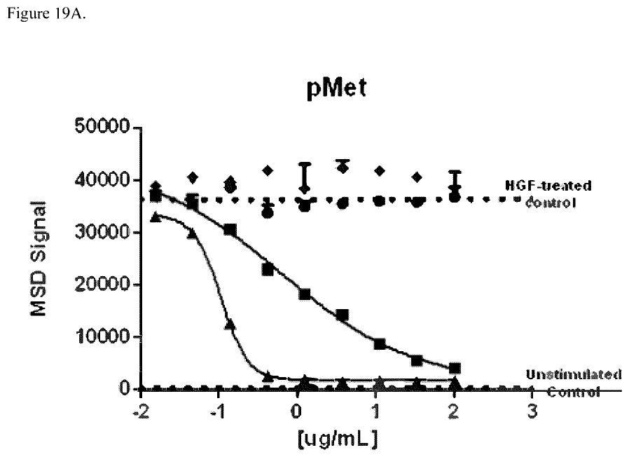

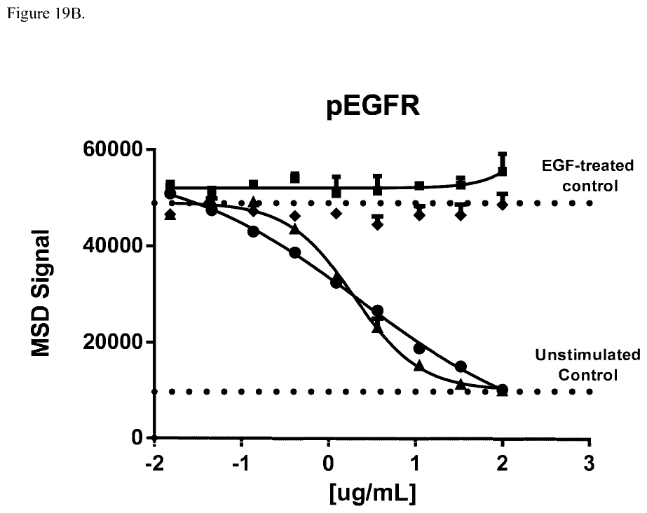

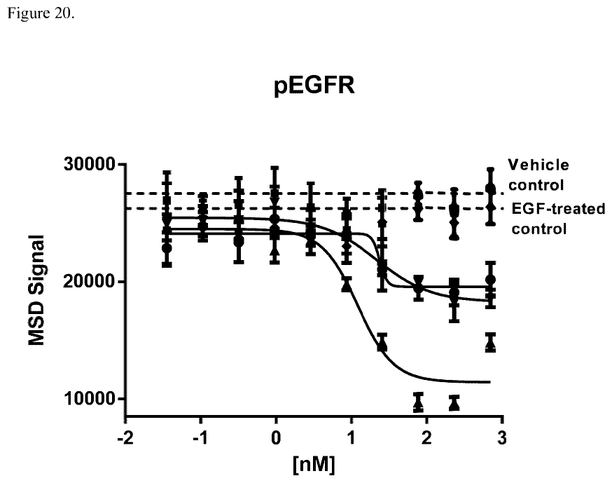

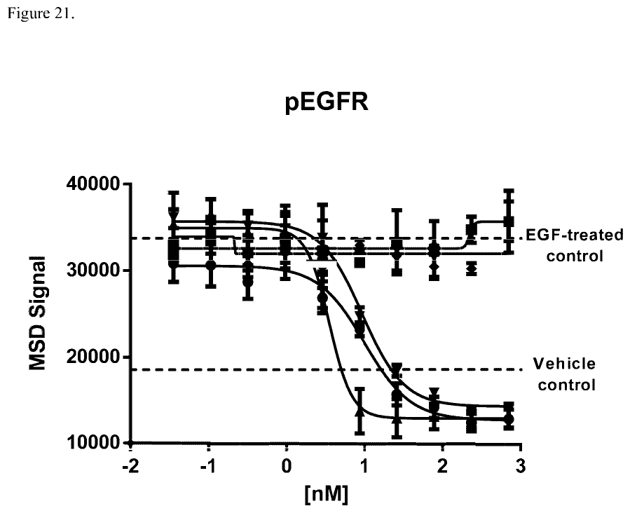

Int. Pat. Publ. No. WO2010/115551 describes a bispecific EGFR/c-Met antibody (BSAB01) engineered in an IgG-scFv format using the EGFR binding VH/VL pair of cetuximab, and the c-Met binding VH/VL pair of an antibody 5D5 (MetMab, onartuzumab) currently in Phase III trials. BSAB01 demonstrates approximately two-fold (additive) increased inhibition of A431 cell proliferation when compared to the parental antibodies (Example 7, FIG. 8b in WO2010/115551), and a modest additive inhibition of Ovarc-8 cell proliferation (FIG. 10a, Example 16 in WO2010/115551) when compared to the combination of the two parental antibodies (15% vs. 10% inhibition). Therefore, surprisingly and unexpectedly, the present invention provides bispecific EGFR/c-Met antibodies that demonstrate a significant synergistic effect in inhibition of EGFR and c-Met signaling, cancer cell survival and tumor growth. By not wishing to be bound by any theory, it is believed that the significant synergistic effect of the bispecific antibodies of the invention at least partially results from the epitope specificity of both the EGFR and the c-Met binding arms, possibly resulting in the inhibition of signaling through not only the EGFR and c-Met homodimers but also the EGFR/HERx heterodimers.

One embodiment of the invention is an isolated bispecific epidermal growth factor receptor (EGFR)/hepatocyte growth factor receptor (c-Met) antibody, comprising:
a) a first heavy chain (HC1) comprising a HC1 constant domain 3 (HC1 CH3) and a HC1 variable region 1 (VH1);
b) a second heavy chain (HC2) comprising a HC2 constant domain 3 (HC2 CH3) and a HC2 variable region 2 (VH2);
c) a first light chain (LC1) comprising a light chain variable region 1 (VL1); and a second light chain (LC2) comprising a light chain variable region 2(VL2),
   wherein the VH1 and the VL1 pair to form a first antigen-binding site that specifically binds EGFR and the VH2 and the VL2 pair to form a second antigen-binding site that specifically binds c-Met, wherein the HC1 comprises at least one substitution in the HC1 CH3 and the HC2 comprises at least one substitution in the HC2 CH3, wherein the substitution in the HC1 CH3 and the substitution in the HC2 CH3 occur at different amino acid residue positions, when residue numbering is according to the EU index.

In some embodiments described herein, the bispecific EGFR/c-Met antibody inhibits phosphorylation of extracellular signal-related kinases 1 and 2 (ERK1/2) in NCI-H292, NCI-H1975 or SKMES-1 cell line with an IC₅₀ value that is at least about 10-fold less, at least about 20-fold less, at least about 30-fold less, at least about 40-fold less, at least about 50-fold less or at least about 60-fold less when compared to the IC₅₀ value of inhibition of phosphorylation of ERK1/2 in NCI-H292, NCI-H1975 or SKMES-1 cell line with a mixture of a control monovalent EGFR antibody comprising a heavy chain 3 (HC3) and a light chain 3 (LC3) and a control monovalent c-Met antibody comprising a heavy chain 4 (HC4) and a light chain 4 (LC4), wherein the HC3 and the HC1, the LC3 and the LC1, the HC4 and the HC2, and the LC4 and the LC2 have identical amino acid sequences, respectively, and the phosphorylation of ERK1/2 is measured in whole cell lysates using a sandwich immunoassay using an anti-phosphoERK1/2 antibody as a capture antibody and an antibody binding to unphosphorylated and phosphorylated ERK1/2 conjugated with an electrochemiluminescent compound as a detection antibody. The bispecific EGFR/c-Met antibodies of the invention provide a synergistic more pronounced inhibition of EGFR and c-Met signaling when compared to the combination of monospecific EGFR antibodies and monospecific c-Met antibodies, when inhibition is assessed by inhibition of ERK1/2 phosphorylation. Such exemplary bispecific EGFR/c-Met antibody is the antibody EM1-mAb of the invention.

“Control monospecific EGFR antibody” as used herein refers to an antibody that has a first Fab arm that binds EGFR that is identical in amino acid sequence to the EGFR-binding Fab arm of the bispecific EGFR/c-Met antibody to be tested, and has a second Fab arm that is “inert” and binds an unrelated/irrelevant antigen, human immunodeficiency virus (HIV) gp120. The second Fab arm has a light chain having the sequence of SEQ ID NO: 209 and a heavy chain having the sequence of SEQ ID NO: 198 in instances when the EGFR binding Fab arm in the bispecific EGFR/c-Met antibody to be tested comprises the F405L substitution. The second Fab arm has a light chain having the sequence of SEQ ID NO: 209 and a heavy chain having the sequence of SEQ ID NO: 197 in instances when the EGFR binding Fab arm in the bispecific EGFR/c-Met antibody to be tested comprises the K409R substitution.

“Control monospecific c-Met antibody” as used herein refers to an antibody that has a first Fab arm that binds c-Met that is identical in amino acid sequence to the c-Met-binding Fab arm of the bispecific EGFR/c-Met antibody to be tested, and has a second Fab arm that is “inert” and binds the unrelated/irrelevant antigen HIV gp120. The second Fab Fab arm has a light chain having the sequence of SEQ ID NO: 209 and a heavy chain having the sequence of SEQ ID NO: 198 in instances when the c-Met binding Fab arm in the bispecific EGFR/c-Met antibody to be tested comprises the F405L substitution. The second inert Fab arm has a light chain having the sequence of SEQ ID NO: 209 and a heavy chain having the sequence of SEQ ID NO: 197 in instances when the c-Met binding Fab arm in the bispecific EGFR/c-Met antibody to be tested comprises the K409R substitution.

In some embodiments described herein, the bispecific EGFR/c-Met antibody inhibits phosphorylation of ERK1/2 with an IC₅₀ value of about 2×10⁻⁹ M or less, about 1×10⁻⁹ M or less, or about 1×10⁻⁸ M or less.

In some embodiments described herein, ERK1 is phosphorylated at residues Thr202 and Tyr204, and ERK2 is phosphorylated at residues Thr185 and Tyr197.

In some embodiments described herein, the bispecific EGFR/c-Met antibody inhibits phosphorylation of protein kinase B (AKT) at Ser473 in NCI-H1975 cell line with an IC₅₀ value that is at least about 70-fold less when compared to the IC₅₀ value of inhibition of phosphorylation of AKT at Ser473 in NCI-H1975 cell line with the mixture of the control monovalent EGFR antibody comprising the HC3 and the LC3 and the control monovalent c-Met antibody comprising the HC4 and the LC4, wherein the HC3 and the HC1, the LC3 and the LC1, the HC4 and the HC2, and the LC4 and the LC2 have identical amino acid sequences, respectively, wherein the phosphorylation of AKT at Ser473 is measured in whole cell lysates using a sandwich immunoassay using an antibody binding to unphosphorylated and phosphorylated AKT as a capture antibody and an anti-phosphoAKT Ser473 antibody conjugated to an electrochemiluminescent compound as a detection antibody.

In some embodiments described herein, the bispecific EGFR/c-Met antibody inhibits phosphorylation of protein kinase B (AKT) at Thr308 in NCI-H1975 cell line with an IC₅₀ value that is at least about 100-fold less when compared to the IC₅₀ value of inhibition of phosphorylation of AKT at Thr308 in NCI-H1975 cell line with the mixture of the control monovalent EGFR antibody comprising the HC3 and the LC3 and the control monovalent c-Met antibody comprising the HC4 and the LC4, wherein the HC3 and the HC1, the LC3 and the LC1, the HC4 and the HC2, and the LC4 and the LC2 have identical amino acid sequences, respectively, wherein the phosphorylation of AKT at Thr308 is measured in whole cell lysates using a sandwich immunoassay using an antibody binding to unphosphorylated and phosphorylated AKT as a capture antibody and an anti-phosphoAKT Thr308 antibody conjugated to an electrochemiluminescent compound as a detection antibody.

The bispecific EGFR/c-Met antibodies of the invention provide a synergistic more pronounced inhibition of EGFR and c-Met signaling when compared to the combination of monospecific EGFR antibodies and monospecific c-Met antibodies, when inhibition is assessed by inhibition of AKT phosphorylation. Such exemplary bispecific EGFR/c-Met antibody is the antibody EM1-mAb of the invention.

In some embodiments described herein, the bispecific EGFR/c-Met antibody inhibits phosphorylation of AKT at Ser473 or at Thr308 with and IC₅₀ value of about 1×10⁻⁹ M or less.

In some embodiments described herein, the bispecific EGFR/c-Met antibody binds EGFR of SEQ ID NO: 73 at EGFR residues K489, I491, K467 and S492 and c-Met at residues PEFRDSYPIKYVHAF (SEQ ID NO: 238) and FAQSKPDSAEPMDRSA (SEQ ID NO: 239). Such an exemplary bispecific antibody is the EM1-mAb. The bispecific EM-1 antibody binds EGFR and c-Met at distinct epitopes when compared to the antibody BSAB01 as described above and in Int. Pat. Publ. No. WO2010/115551. The parental EGFR binding arm (cetuximab) of BSAB01 binds EGFR amino acid residues R353, Q384, Q408, H409, F412, S418, S440, K443, K465, 1467, S468, and N473 in mature EGFR, corresponding to residues R367, Q408, Q432, H433, F436, S442, S464, K467, K489, I491, S492 and N497 of full length EGFR of SEQ ID NO: 73 (Li et al., Cancer Cell 7:301-311, 2005). The parental c-Met binding arm of BSAB01 (mAb 5D5) binds c-Met residues 325-340 PGAQLARQIGASLNDD (SEQ ID NO: 240). Epitope mapping of the EGFR binding parental antibody (2F8) of the EM1-mAb is described in US. Pat. Publ. No. US2011/0256142A1. Cetuximab and the parental 2F8 antibody bind partially overlapping but distinct epitopes.

Epitope mapping can be done using standard methods. For example, when the structures of both individual components are known, in silico protein-protein docking can be carried out to identify compatible sites of interaction. Hydrogen-deuterium (H/D) exchange can be carried out with the antigen and antibody complex to map regions on the antigen that may be bound by the antibody. Segment and point mutagenesis of the antigen can be used to locate amino acids important for antibody binding.

In some embodiments described herein, the bispecific EGFR/c-Met antibody neutralizes EGFR and c-Met signaling.

The bispecific EGFR/c-Met antibody of the invention may neutralize EGFR and c-Met signaling by at least 30%, 35%, 40%, 45%, 50%, 55%, 60%, 65%, 70%, 75%, 80%, 85%, 90%6, 91%, 92%, 93%, 94%, 95%, 96%, 97%, 98%, 99% or 100% when compared to the level of signaling in the absence of the bispecific EGFR/c-Met molecule of the invention using the same assay conditions.

Binding of a ligand such as EGF to EGFR stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division. Neutralization of EGFR signaling may result in inhibition in one or more EGFR downstream signaling pathways and therefore neutralizing EGFR may have various effects, including inhibition of cell proliferation and differentiation, angiogenesis, cell motility and metastasis, and inhibition of downstream signaling pathways.

EGFR signaling and neutralization of EGFR signaling may be measured using various well know methods, for example measuring the autophosphorylation of the receptor at any of the tyrosines Y1068, Y1148, and Y1173 (Downward et al., Nature 311:483-5, 1984) and/or phosphorylation of natural or synthetic substrates, and inhibition of autophosphorylation and/or phosphorylation of natural or synthetic substrates by the bispecific antibodies of the invention. Phosphorylation can be detected using well known methods such as an ELISA assay or a western plot using a phosphotyrosine specific antibody. Exemplary assays can be found in Panek et al., J Pharmacol Exp Them 283:1433-44, 1997 and Batley et al., Life Sci 62:143-50, 1998, and as described herein.

Binding of HGF to c-Met stimulates receptor dimerization, autophosphorylation, activation of the receptor's cytoplasmic tyrosine kinase domain, and initiation of multiple signal transduction and transactivation pathways involved in regulation of DNA synthesis (gene activation) and cell cycle progression or division. Inhibition of c-Met signaling may result in inhibition in one or more c-Met downstream signaling pathways and therefore neutralizing c-Met may have various effects, including inhibition of cell proliferation and differentiation, angiogenesis, cell motility and metastasis.

c-Met signaling and neutralization of c-Met signaling may be measured using various well know methods, for example measuring the autophosphorylation of the receptor on at least one tyrosine residues Y1230, Y1234, Y1235 or Y1349, and/or phosphorylation of natural or synthetic substrates. Phosphorylation can be detected, for example, using an antibody specific for phosphotyrosine in an ELISA assay or on a western blot. Exemplary assays can be found in Panek et al., J Pharmacol Exp Thera 283:1433-44, 1997 and Batley et al., Life Sci 62:143-50, 1998, and as described herein.

EGFR and c-Met signaling may be measured using various well know methods as described herein, such as measuring inhibition of ERK1/2 and AKT phosphorylation. Inhibition of ERK1 phosphorylation at Thr202 and Tyr204 and ERK2 phosphorylation at Thr185 and Tyr187 and inhibition of AKT at Ser473 or Thr308 can be measured for example in NCI-H1975 cell lysates utilizing a sandwich assay with capture antibody coated on solid support, and the detection antibody conjugated with an electrochemiluminescent compound such as Meso Scale Discover (MSD) SULFO-TAG label, followed by detection of the signal with a plate reader.

In some embodiments described herein, the bispecific EGFR/c-Met antibody inhibits growth of NCI-H292 or NCI-H1975 cells with an IC₅₀ value that is at least about 300-fold less, at least about 400-fold less, at least about 500-fold less, at least about 600-fold less, at least about 700-fold less or at least about 800-fold less when compared to the IC₅₀ value of inhibition of growth of NCI-H292 or NCI-H1975 cells with cetuximab, when NCI-H292 or NCI-H1975 cells are grown in low attachment conditions.

Inhibition of cell growth may be assessed by known methods. For example, the cells may be plated in plates coated with hydrogels or biomimetic polymers (for example Ultra Low Attachment plates by Corning) to prevent or reduce cell attachment, and the effect of antibodies on 7.5 ng/mL HGF-induced cell growth can be assessed by measuring percent cell viability after incubation for 72 hours using standard methods.

The bispecific EGFR/c-Met antibodies of the invention provide a synergistic more pronounced inhibition of EGFR and/or c-Met expressing cancer cells when compared to the combination of monospecific EGFR antibodies and monospecific c-Met antibodies and to the standard of care cetuximab. Such an exemplary bispecific EGFR/c-Met antibody is the antibody EM1-mAb of the invention. The bispecific EGFR/c-Met antibodies of the invention inhibit cancer cells that express the wild type EGFR and the wild type c-Met, and also cancer cells that express the EGFR L858R/T790M mutant, which mutation is identified to contribute to resistance to treatments with small molecule tyrosine kinase inhibitors (TKIs) such as gefitinib. Therefore the bispecific EGFR/c-Met antibodies of the invention may provide a benefit in a broader patient population when compared to cetuximab and TKIs.

In some embodiments described herein, the bispecific EGFR/c-Met antibody inhibits growth of HGF-expressing SKMES-1 cell tumor in SCID Beige mice with a percentage (%) T/C value of at least 500-fold less on day 36 when compared to cetuximab, when the bispecific antibody and cetuximab are administered at 20 mg/kg dose.

Tumor xenograft models using SCID Beige mice are well known. SKMES-1 cells may be engineered to express human HGF using standard methods. Typically, SCID Beige mice may be subcutaneously inoculated with SKMES-1 cells expressing human HOF embedded in extracellular matrix such as Culturex in the dorsal flank of each animal. One week after implantation, mice may be stratified into groups with equivalent tumor volumes, and thereafter dosed for example three times per week with the bispecific EGFR/c-Met antibodies of the invention, control or benchmark antibodies or small molecules. Tumor volumes may be recorded twice weekly, and tumor growth inhibition (TGI) may be observed by calculating the percentage (%) T/C value. The % T/C value is indicative of anti-tumor efficacy. T and C are the mean volumes of the treated and control groups, respectively, on a given day.

The bispecific EGFR/c-Met antibodies of the invention provide a significantly improved efficacy in in vivo tumor killing when compared to the standard of care cetuximab, and therefore may provide a benefit in a patient population when compared to cetuximab.

In some embodiments described herein, the bispecific EGFR/c-Met antibody heterodimerizes EGFR and c-Met on the cell surface. While not wishing to be bound by any particular theory, it is believed that the binding of the antibody of the invention to EGFR homodimers, c-Met homodimers, and EGFR/c-Met heterodimers results in simultaneous inhibition of ligand mediated signaling downstream of EGFR homodimers, c-Met homodimers and EGFR/c-Met heterodimers, providing synergistic inhibition when compared to the combination of monospecific anti-EGFR and anti-c-Met antibodies.

In some embodiments described herein, the bispecific EGFR/c-Met antibody comprises the HC1 and the HC2 of IgG1, IgG2. IgG3 or IgG4 isotype.

In some embodiments described herein, the bispecific EGFR/c-Met antibody comprises the HC1 and the HC2 of IgG1 isotype.

In some embodiments described herein, the bispecific EGFR/c-Met antibody HC1 CH3 comprises at least one, two, three, four, five, six, seven or eight substitutions and the HC2 CH3 comprises at least one, two, three, four, five, six, seven or eight substitutions at residue positions 350, 366, 368, 370, 399, 405, 407 or 409, when residue numbering is according to the EU index.

In some embodiments described herein, the bispecific EGFR/c-Met antibody HC1 CH3 comprises at least one, two, three or four substitutions and the HC2 CH3 comprises at least one, two, three or four substitutions at residue positions 350, 370, 405 or 409, when residue numbering is according to the EU index.

Antibody domains and numbering are well known. Two CH3 domains (or CH3 regions) are non-identical when they differ with at least one amino acid substitution from each other. An IgG1 CH3 region typically consists of residues 341-446 on IgG1 (residue numbering according to the EU index). An exemplary IgG1 constant region is shown in SEQ ID NO: 203. The CH3 domain spans residues 224-329 of SEQ ID NO: 203, and correspond to residues 341-446 according to EU index.

In some embodiments described herein, the bispecific EGFR/c-Met antibody HC1 CH3 comprises at least one substitution and the HC2 CH3 comprises at least one substitution at residue positions 405 or 409, when residue numbering is according to the EU index.

In some embodiments described herein, the bispecific EGFR/c-Met antibody HC1 CH3 comprises a K409R or a F405L substitution and the HC2 CH3 comprises a K409R or a F405L substitution, wherein residue numbering is according to the EU index.

In some embodiments described herein, the bispecific EGFR/c-Met antibody HC1 CH3 comprises the F405L substitution and the HC2 CH3 comprises the K409R substitution.

In some embodiments described herein, the HC1 CH3 and the HC2 CH3 substitutions are substitutions at position 366, 368, 370, 399, 405, 407 or 409 (numbering according to the EU index). These positions correspond to linear residue positions 248, 250, 252, 281, 287, 289 and 291, respectively, in a heavy chain constant region of SEQ ID NO: 203 and 204.

In some embodiments described herein, the HC1 CH3 position 409 has an amino acid substitution other than Lys, Leu or Met and the HC2 CH3 position 405 has an amino acid substitution other than Phe.

In some embodiments described herein, the HC1 CH3 position 405 has an amino acid substitution other than Phe and the HC2 CH3 position 409 has an amino acid substitution other than Lys, Leu or Met.

In some embodiments described herein, the HC1 CH3 position 409 has an amino acid substitution other than Lys, Leu or Met and the HC2 CH3 position 405 has an amino acid substitution other than Phe, Arg or Gly.

In some embodiments described herein, the HC1 CH3 position 405 has an amino acid substitution other than Phe, Arg or Gly and the HC2 CH3 position 409 has an amino acid substitution other than Lys, Leu or Met

In some embodiments described herein, the HC1 CH3 has Phe at position 405 and an amino acid other than Lys, Leu or Met at position 409 and the HC2 CH3 has an amino acid other than Phe at position 405 and a Lys at position 409.

In some embodiments described herein, the HC1 CH3 has an amino acid other than Phe at position 405 and Lys at position 409 and the HC2 CH3 has Phe at position 405 and an amino acid other than Lys, Leu or Met at position 409.

In some embodiments described herein, the HC1 CH3 has Phe at position 405 and an amino acid other than Lys, Leu or Met at position 409 and the HC2 CH3 has a substitution other than Phe. Arg or Gly at position 405 and Lys at position 409.

In some embodiments described herein, the HC1 CH3 has a substitution other than Phe, Arg or Gly at position 405 and Lys at position 409 and the HC2 CH3 has Phe at position 405 and an amino acid other than Lys, Leu or Met at position 409.

In some embodiments described herein, the HC1 CH3 has Phe at position 405 and an amino acid other than Lys, Leu or Met at position 409 and the HC2 CH3 has Leu at position 405 and Lys at position 409.

In some embodiments described herein, the HC1 CH3 has Leu at position 405 and Lys at position 409 and the HC2 CH3 has Phe at position 405 and an amino acid other than Lys, Leu or Met at position 409.

In some embodiments described herein, the HC1 CH3 has Phe at position 405 and aArg at position 409 and the HC2 CH3 has an amino acid other than Phe, Arg or Gly at position 405 and Lys at position 409.

In some embodiments described herein, the HC1 CH3 has an amino acid other than Phe, Arg or Gly at position 405 and Lys at position 409 and the HC2 CH3 has Phe at position 405 and Arg at position 409.

In some embodiments described herein, the HC1 CH3 has Phe at position 405 and Arg at position 409 and the HC2 CH3 has Leu at position 405 and Lys at position 409.

In some embodiments described herein, the HC1 CH3 has Leu at position 405 and Lys at position 409 and the HC2 CH3 has Phe at position 405 and Arg at position 409.

In some embodiments described herein, the HC1 CH3 has Phe at position 405 and Lys at position 409 and the HC2 CH3 has Leu at position 405 and aArg at position 409.

In some embodiments described herein, the HC1 CH3 has Leu at position 405 and aArg at position 409 and the HC2 CH3 has Phe at position 405 and Lys at position 409.

In some embodiments described herein, the HC1 CH3 has an amino acid other than Lys, Leu or Met at position 409 and the HC2 CH3 has Lys at position 409, Thr at position 370 and Leu at position 405.

In some embodiments described herein, the HC1 CH3 has Lys at position 409, Thr at position 370 and Leu at position 405 and the HC2 CH3 has an amino acid other than Lys, Leu or Met at position 409.

In some embodiments described herein, the HC1 CH3 has Arg at position 409 and the HC2 CH3 has Lys at position 409, Thr at position 370 and Leu at position 405.

In some embodiments described herein, the HC1 CH3 has Lys at position 409, Thr at position 370 and Leu at position 405 and the HC2 CH3 has Arg at position 409.

In some embodiments described herein, the HC1 CH3 has Lys at position 370, Phe at position 405 and aArg at position 409 and the HC2 CH3 has Lys at position 409, Thr at position 370 and Leu at position 405.

In some embodiments described herein, the HC1 CH3 has Lys at position 409, Thr at position 370 and Leu at position 405 and the HC2 CH3 has Lys at position 370, Phe at position 405 and Arg at position 409.

In some embodiments described herein, the HC1 CH3 has an amino acid other than Lys, Leu or Met at position 409 and the HC2 CH3 has an amino acid other than Tyr, Asp, Glu, Phe, Lys, Gln, Arg, Ser or Thr at position 407.

In some embodiments described herein, the HC1 CH3 has an amino acid other than Tyr, Asp, Glu, Phe, Lys, Gln, Arg, Ser or Thr at position 407 and the HC2 CH3 has an amino acid other than Lys, Leu or Met at position 409.

In some embodiments described herein, the HC1 CH3 has an amino acid other than Lys, Leu or Met at position 409 and the HC2 CH3 has Ala, Gly, His, Ile, Leu, Met, Asn, Val or Trp at position 407.

In some embodiments described herein, the HC1 CH3 has Ala, Gly, His, Ile, Leu, Met, Asn, Val or Trp at position 407 and the HC2 CH3 has an amino acid other than Lys, Leu or Met at position 409.

In some embodiments described herein, the HC1 CH3 has an amino acid other than Lys, Leu or Met at position 409 and the HC2 CH3 has Gly, Leu, Met, Asn or Trp at position 407.

In some embodiments described herein, the HC1 CH3 has Gly, Leu, Met, Asn or Trp at position 407 and the HC2 CH3 has an amino acid other than Lys, Leu or Met at position 409.

In some embodiments described herein, the HC1 CH3 has Tyr at position 407 and an amino acid other than Lys, Leu or Met at position 409 and the HC2 CH3 has an amino acid other than Tyr, Asp, Glu, Phe, Lys, Gln, Arg, Ser or Thr at position 407 and Lys at position 409.

In some embodiments described herein, the HC1 CH3 has an amino acid other than Tyr, Asp, Glu, Phe, Lys, Gln, Arg, Ser or Thr at position 407 and Lys at position 409 and the HC2 CH3 has Tyr at position 407 and an amino acid other than Lys, Leu or Met at position 409.

In some embodiments described herein, the HC1 CH3 has Tyr at position 407 and an amino acid other than Lys, Leu or Met at position 409 and the HC2 CH3 has Ala, Gly.

His, Ile, Leu, Met, Asn, Val or Trp at position 407 and Lys at position 409.

In some embodiments described herein, the HC1 CH3 has Ala, Gly, His, Ile, Leu, Met, Asn, Val or Trp at position 407 and Lys at position 409 and the HC2 CH3 has Tyr at position 407 and an amino acid other than Lys, Leu or Met at position 409.

In some embodiments described herein, the HC1 CH3 has Tyr at position 407 and an amino acid other than Lys, Leu or Met at position 409 and the HC2 CH3 has Gly, Leu, Met, Asn or Trp at position 407 and Lys at position 409.

In some embodiments described herein, the HC1 CH3 has Gly, Leu, Met, Asn or Trp at position 407 and Lys at position 409 and the HC2 CH3 has Tyr at position 407 and an amino acid other than Lys, Leu or Met at position 409.

In some embodiments described herein, the HC1 CH3 has Tyr at position 407 and Arg at position 409 and the HC2 CH3 has an amino acid other than Tyr, Asp, Glu, Phe, Lys, Gln, Arg, Ser or Thr at position 407 and Lys at position 409.

In some embodiments described herein, the HC1 CH3 has an amino acid other than Tyr, Asp, Glu, Phe, Lys, Gln, Arg, Ser or Thr at position 407 and Lys at position 409 and the HC2 CH3 has Tyr at position 407 and Arg at position 409.

In some embodiments described herein, the HC1 CH3 has Tyr at position 407 and Arg at position 409 and the HC2 CH3 has Ala, Gly, His, Ile. Leu, Met, Asn, Val or Trp at position 407 and Lys at position 409.

In some embodiments described herein, the HC1 CH3 has Ala, Gly, His, Ile, Leu, Met, Asn, Val or Trp at position 407 and Lys at position 409 and the HC2 CH3 has Tyr at position 407 and Arg at position 409.

In some embodiments described herein, the HC1 CH3 has Tyr at position 407 and Arg at position 409 and the HC2 CH3 has Gly, Leu, Met, Asn or Trp at position 407 and Lys at position 409.

In some embodiments described herein, the HC1 CH3 has Gly, Leu, Met, Asn or Trp at position 407 and Lys at position 409 and the HC2 CH3 has Tyr at position 407 and Arg at position 409.

In some embodiments described herein, the HC1 CH3 has an amino acid other than Lys, Leu or Met at position 409, and the HC2 CH3 has (i) an amino acid other than Phe, Leu and Met at position 368, or (ii) a Trp at position 370, or (iii) an amino acid other than Asp, Cys, Pro, Glu or Gln at position 399.

In some embodiments described herein, the HC1 CH3 has (i) an amino acid other than Phe, Leu and Met at position 368, or (ii) a Trp at position 370, or (iii) an amino acid other than Asp, Cys, Pro, Glu or Gln at position 399 and the HC2 CH3 has an amino acid other than Lys, Leu or Met at position 409.

In some embodiments described herein, the HC1 CH3 has Arg, Ala, His or Gly at position 409, and the HC2 CH3 has (i) Lys, Gln, Ala, Asp, Glu, Gly, His, Ile, Asn, Arg, Ser, Thr, Val, or Trp at position 368, or (ii) Trp at position 370, or (iii) Ala, Gly, Ile, Lou, Met, Asn, Ser, Thr, Trp, Phe, His, Lys. Arg or Tyr at position 399.

In some embodiments described herein, the HC1 CH3 has (i) Lys, Gln, Ala, Asp, Glu, Gly, His, Ile, Asn, Arg, Ser, Thr, Val, or Trp at position 368, or (ii) Trp at position 370, or (iii) Ala, Gly, Ile, Leu, Met, Asn, Ser, Thr, Tip, Phe, His, Lys, Arg or Tyr at position 399 and the HC2 CH3 has Arg, Ala, His or Gly at position 409.

In some embodiments described herein, the HC1 CH3 has Arg at position 409, and the HC2 CH3 has (i) Asp, Glu, Gly, Asn, Arg, Ser, Thr, Val, or Trp at position 368, or (ii) Trp at position 370, or (iii) Phe, His, Lys, Arg or Tyr at position 399.

In some embodiments described herein, the HC1 CH3 has (i) Asp, Glu, Gly, Asn, Arg, Ser, Thr, Val, or Trp at position 368, or (ii) Trp at position 370, or (iii) Phe, His, Lys, Arg or Tyr at position 399 and the HC2 CH3 has Arg at position 409.

In some embodiments described herein, the HC1 CH3 comprises a K409R substitution or a F405L substitution and the HC2 CH3 comprises a K409R substitution or a F40SL substitution, wherein the residue numbering is according to the EU index.

In some embodiments described herein, the HC1 CH3 comprises the F405L substitution and the HC2 CH3 comprises the K409R substitution.

Substitutions are typically made at the DNA level to a molecule such as the constant domain of the antibody using standard methods.

In some embodiments described herein, the bispecific EGFR/c-Met antibody comprises the VH1 and the VL1, wherein
the VH1 comprises the heavy chain complementarity determining region (HCDR) 1 (HCDR1), HCDR 2 (HCDR2) and HCDR 3 (HCDR3) amino acid sequences of SEQ ID NOs: 210, 211 and 212, respectively; and
the VL1 comprises the light chain complementarity determining region (LCDR) 1 (LCDR1), LCDR 2 (LCDR2) and LCDR 3 (LCDR3) amino acid sequences of SEQ ID NOs: 213, 214 and 215, respectively.

In some embodiments described herein, the bispecific EGFR/c-Met antibody comprises the VH2 and the VL2, wherein
the VH2 comprises the HCDR1, the HCDR2, and the HCDR3 amino acid sequences of SEQ ID NOs: 216, 217 and 218, respectively; and
the VL2 comprises the LCDR1, the LCDR2 and the LCDR3 amino acid sequences of SEQ ID NOs: 219, 220 and 221, respectively.

In some embodiments described herein, the bispecific EGFR/c-Met antibody comprises the VH1, the VL1, the VH2 and the VL2 amino acid sequences of SEQ ID NOs: 189, 190, 193 and 194, respectively.

In some embodiments described herein, the bispecific EGFR/c-Met antibody comprises the HC1, the LC1, the HC2 and the LC2 amino acid sequences of SEQ ID NOs:
199, 200, 201 and 202, respectively, optionally having a C-terminal lysine removed from the HC1, the HC2, or both the HC1 and the HC2.

In some embodiments described herein, the bispecific EGFR/c-Met antibody comprises the VH1 and the VL1, wherein
the VH1 comprises the HCDR1, the HCDR2, and the HCDR3 amino acid sequences of SEQ ID NOs: 222, 223 and 224, respectively; and
the VL1 comprises the LCDR1, the LCDR2 and the LCDR3 amino acid sequences of SEQ ID NOs: 225, 226 and 227, respectively.

In some embodiments described herein, the bispecific EGFR/c-Met antibody comprises the VH2 and the VL2, wherein
the V112 comprises the HCDR1, the HCDR2, and the HCDR3 amino acid sequences of SEQ ID NOs: 228,229 and 230, respectively; and
the VL2 comprises the LCDR1, the LCDR2 and the LCDR3 amino acid sequences of SEQ ID NOs: 231, 232 and 233, respectively.

In some embodiments described herein, the bispecific EGFR/c-Met antibody comprises the VH1, the VL1, the VH2 and the VL2 amino acid sequences of SEQ ID NOs: 191, 192, 195 and 196, respectively.

In some embodiments described herein, the bispecific EGFR/c-Met antibody comprises the HC1, the LC1, the HC2 and the LC2 amino acid sequences of SEQ ID NOs: 234, 235, 236 and 237, respectively, optionally having the C-terminal lysine removed from the HC1, the HC2, or both the HC1 and the HC2.

In some embodiments described herein, the bispecific EGFR/c-Met antibodies may block EGF binding to the EGFR and HGF binding to c-Met with an IC₅₀ value of less than about 1×10⁻⁸ M, less than about 1×10⁻⁹ M, less than about 1×10⁻¹⁰ M, less than about 1×10⁻¹¹ M, or less than about 1×10⁻¹² M in a competition assay employing recombinant human EGFR or recombinant human c-Met extracellular domains coated on plates and incubated with or without the bispecific EGFR/c-Met antibodies of the invention. The bispecific EGFR/c-Met antibodies described herein may block EGF binding to EGFR and HGF binding to c-Met by at least 30%, 35%, 40%, 45%, 50%, 55%, 60%, 65%, 70%, 75%, 80%, 85%, 90%/e, 91%, 92%, 93%, 94%, 95%, 96%, 97%, 98%, 99% or 100% when compared to binding of EGF to the EGFR and HGF binding to c-Met in the absence of the bispecific EGFR/c-Met antibodies of the invention described herein using the same assay conditions.

In some embodiments described herein, the bispecific EGFR/c-Met antibody comprises the HC1, LC1, HC2 and LC2, wherein the HC1, the LC1, the HC2 and the LC2 are encoded by synthetic polynucleotides comprising the sequence of SEQ ID NOs: 205, 206, 207 and 208, respectively.

The bispecific EGFR/c-Met antibodies of the invention may be generated using techniques described herein, such as utilizing CH3 engineering and generating the antibodies using in vitro Fab arm exchange. An exemplary bispecific antibody may be generated from two monospecific antibodies by combining about 1-20 mg/mL of each antibody at a 1:1 molar ratio in PBS at pH 7.0-7.4 in a buffer having a final concentration of 75 mM 2-mercaptoethanolamine (2-MEA), incubating for 2-6 hours at 25-37° C., followed by removal of 2-MEA via dialysis, diafiltration, tangential flow filtration, and spinned cell filtration. The yield of the bispecific antibody may be more than about 80%, more than about 90%, more than about 91%, 92%, 93%, 94%, 95%, 96%, 97%, 98% or 99%.

Some embodiments described herein provide for methods of producing the isolated bispecific EGFR/c-Met antibody, comprising:
combining an isolated monospecific bivalent anti-EGFR antibody comprising two heavy chains of SEQ ID NO: 199 and two light chains of SEQ ID NO: 200 and an isolated monospecific bivalent anti-c-Met antibody comprising two heavy chains of SEQ ID NO: 201 and two light chains of SEQ ID NO: 202 in a mixture of about 1:1 molar ratio;
introducing a reducing agent into the mixture;
incubating the mixture about ninety minutes to about six hours;
removing the reducing agent; and
purifying the bispecific EGFR/c-Met antibody that comprises a first heavy chain of SEQ ID NO: 199 and a second heavy chain of SEQ ID NO: 201, a first light chain of SEQ ID NO: 200 and a second light chain of SEQ ID NO: 202, wherein the first heavy chain of SEQ ID NO: 199 pairs with the first light chain of SEQ ID NO: 200 to form the first binding domain that specifically binds EGFR, and the second heavy chain of SEQ ID NO: 201 pairs with the second light chain of SEQ ID NO: 202 to form the second binding domain that specifically binds c-Met.

In some embodiments described herein, the reducing agent is 2-mercaptoethanolamine (2-MEA).

In some embodiments described herein, 2-MEA is present at a concentration of about 25 mM to about 75 mM.

In some embodiments described herein, the incubating step is performed at a temperature of about 25° C. to about 37° C.

Some embodiments described herein provide for an isolated bispecific EGFR/-c-Met antibody comprising a HC1, a LC1, a HC2 and a LC2, wherein the HC1 comprises the sequence of SEQ ID NO: 199, the LC1 comprises the sequence of SEQ ID NO: 200, the HC2 comprises the sequence of SEQ ID NO: 201, and the LC2 comprises the sequence of SEQ ID NO: 202, wherein the HC1, the LC1, the HC2 and/or the LC2 further comprises 1, 2, 3, 4, 5, 6, 7, 8, 9, 10, 11, 12, 13, 14 or 15 conservative amino acid substitutions.

Some embodiments described herein provide for an isolated bispecific EGFR/-c-Met antibody comprising the HC1, the LC1, the HC2 and the LC2, wherein the HC1 comprises the sequence of SEQ ID NO: 234, the LC1 comprises the sequence of SEQ ID NO: 235, the HC2 comprises the sequence of SEQ ID NO: 236, and the LC2 comprises the sequence of SEQ ID NO: 237, wherein the HC1, the LC1, the HC2 and/or the LC2 further comprises 1, 2, 3, 4, 5, 6, 7, 8, 9, 10, 11, 12, 13, 14 or 15 conservative amino acid substitutions.

Bispecific EGFR/c-Met antibodies whose HC1, LC1, HC2 and LC2 amino acid sequences differ insubstantially from those antibodies disclosed herein are encompassed within the scope of the invention. Typically, this involves one or more conservative amino acid substitutions with an amino acid having similar charge, hydrophobic, or stereochemical characteristics in the antigen-binding sites or in the frameworks without adversely altering the properties of the antibody. Conservative substitutions may also be made to improve antibody properties, for example stability or affinity. 1, 2, 3, 4, 5, 6, 7, 8, 9, 10, 11, 12, 13, 14, or 15 amino acid substitutions may be made for example to the VH1, the VL1, the V112 and/or the VL2. For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for alanine scanning mutagenesis (MacLennan et al., Acta Physiol Scand Suppl643:55-67, 1998; Sasaki et al., Adv Biophys 35:1-24, 1998). Desired amino acid substitutions may be determined by those skilled in the art at the time such substitutions are desired. For example, amino acid substitutions can be used to identify important residues of the molecule sequence, or to increase or decrease the affinity of the molecules described herein. Exemplary conservative amino acid substitutions are described supra.

Amino acid substitutions may be done for example by PCR mutagenesis (U.S. Pat. No. 4,683,195). Libraries of variants may be generated using well known methods, for example using random (NNK) or non-random codons, for example DVK codons, which encode 11 amino acids (Ala, Cys, Asp, Glu, Gly, Lys, Asn, Arg, Ser, Tyr, Trp) and screening the libraries for variants with desired properties.

In some embodiments described herein, amino acid substitutions can be made to the constant region of the antibody. For example different IgG1 allotypes can be used in the bispecific EGFR/c-Met antibodies of the invention, such as well known G1m17 allotype, G1m3 allotype or G1m1 allotype, or a combination thereof.

In some embodiments described herein pharmacokinetic properties of the bispecific EGFR/c-Met antibodies may be enhanced by substitutions in the Fc domain that modulate antibody half-life. In some embodiments described herein, the bispecific EGFR/c-Met antibody comprises a substitution M252Y/S254T/T256E in the HC1 and/or the HC2, wherein residue numbering is according to the EU index. M252Y/S254T/T256E substitutions have been show to increase antibody half-life (Dall'Acqua et al., J Biol Chem 281:23514-24, 2006).

The bispecific EGFR/c-Met antibodies having conservative substitutions and/or additional substitutions in their Fc region are tested for their characteristics using the methods described herein.

In some embodiment described herein, immune effector properties of the bispecific EGFR/c-Met antibodies may be enhanced or silenced through Fc modifications by techniques known to those skilled in the art. For example, Fc effector functions such as C1q binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor, BCR), etc. may be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of monoclonal antibodies to induce ADCC can be enhanced by engineering their oligosaccharide component. Human IgG1 or IgG3 are N-glycosylated at Asn297 with the majority of the glycans in the well known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such mAbs can be achieved using different methods reported to lead to the successful expression of relatively high defucosylated antibodies bearing the biantennary complex-type of Fc oligosaccharides such as control of culture osmolality (Konno et al., Cytotechnology 64(249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α 1,6-fucosyltransferase (FUT8) gene (Mori et al., Biotechnol Bioeng 88:901-908, 2004), or coexpression of β-1,4-N-acetylglucosaminyltransferase III and Golgi α-mannosidase 11 or a potent alpha-mannosidase I inhibitor, kifinensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein. ADCC elicited by the bispecific EGFR/c-Met antibodies may also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

In some embodiments described herein, the bispecific EGFR/c-Met antibody of the invention has a biantennary glycan structure with fucose content of about between 1% to about 15%, for example 15%, 14%, 13%, 12%, 11% 10%, 9%, 8%, 7%, 6%, 5%, 4%, 3%, 2% or 1% In some embodiments, the bispecific EGFR/c-Met antibody has a glycan structure with fucose content of about 50%, 40%, 45%, 40%, 35%, 30%, 25%, or 20%.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Int Pat. Publ. No. WO2008/077546 2); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used in the application refers to antibodies with fucose content of about between 1%-15%.

“Normal fucose” or “normal fucose content” as used herein refers to antibodies with fucose content of about over 50%, typically about over 80% or over 85%.

Some embodiments of the invention provide a synthetic nucleic acid encoding the heavy chains and the light chains of the bispecific EGFR/c-Met binding antibodies of the invention as described herein as isolated polynucleotides or as portions of expression vectors or as portions of linear DNA sequences, including linear DNA sequences used for in vitro transcription/translation, vectors compatible with prokaryotic, eukaryotic or filamentous phage expression, secretion and/or display of the compositions or directed mutagens thereof.

Some embodiments of the invention provide an isolated polynucleotide comprising the polynucleotide sequence of SEQ ID NOs: 205, 206, 207 or 208.

The polynucleotides of the invention may be produced by chemical synthesis such as solid phase polynucleotide synthesis on an automated polynucleotide synthesizer and assembled into complete single or double stranded molecules. Alternatively, the polynucleotides of the invention may be produced by other techniques such as PCR followed by routine cloning. Techniques for producing or obtaining polynucleotides of a given known sequence are well known in the art.

The polynucleotides of the invention may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

Some embodiments described herein provide for a vector comprising the polynucleotide of the invention. Such vectors may be plasmid vectors, viral vectors, vectors for baculovirus expression, transposon based vectors or any other vector suitable for introduction of the polynucleotide of the invention into a given organism or genetic background by any means. For example, polynucleotides encoding heavy and light chains of the bispecific antibodies of the invention may be inserted into expression vectors. The light and heavy chains may be cloned in the same or different expression vectors. The DNA segments encoding immunoglobulin chains may be operably linked to control sequences in the expression vector(s) that ensure the expression of immunoglobulin polypeptides. Such control sequences include signal sequences, promoters (e.g. naturally associated or heterologous promoters), enhancer elements, and transcription termination sequences, and may be chosen to be compatible with the host cell chosen to express the antibody. Once the vector has been incorporated into the appropriate host, the host may be maintained under conditions suitable for high level expression of the proteins encoded by the incorporated synthetic polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance or neomycin resistance to permit detection of those cells transformed with the desired DNA sequences.

Some embodiments described herein provide for a host cell comprising the vector of the invention. The term “host cell” refers to a cell into which a vector has been introduced. It is understood that the term host cell is intended to refer not only to the particular subject cell but to the progeny of such a cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells.

Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NS0 (European Collection of Cell Cultures (ECACC). Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics. Walkersville, MD), CHO-K1 (ATCC CRL-61) or DG44.

Uses of Bispecific EGFR/c-Met FN3 Domain Containing Molecules, Bispecific EGFR/-c-Met Antibodies and EGFR-Binding or c-Met Binding FN3 Domains of the Invention

The bispecific EGFR/c-Met FN3 domain containing molecules, the EGFR binding FN3 domains, the c-Met binding FN3 domains or the bispecific EGFR-c-Met antibodies of the invention may be used to diagnose, monitor, modulate, treat, alleviate, help prevent the incidence of, or reduce the symptoms of human disease or specific pathologies in cells, tissues, organs, fluid, or, generally, a host. The methods of the invention may be used to treat an animal patient belonging to any classification. Examples of such animals include mammals such as humans, rodents, dogs, cats and farm/domestic animals.

One aspect of the invention is a method for inhibiting growth or proliferation of cells that express EGFR and/or c-Met, comprising contacting the cells with the isolated bispecific EGFR/c-Met FN3 domain containing molecule, the EGFR binding FN3 domain, the c-Met binding FN3 domain or the bispecific EGFR/c-Met antibody of the invention.

Another aspect of the invention is a method for inhibiting growth or metastasis of EGFR and/or c-Met-expressing tumor or cancer cells in a subject comprising administering to the subject an effective amount of the isolated bispecific EGFR/c-Met FN3 domain containing molecule, the EGFR binding FN3 domain, the c-Met binding FN3 domain or the bispecific EGFR/c-Met antibody of the invention so that the growth or metastasis of EGFR- and/or c-Met-expressing tumor or cancer cell is inhibited.

Another aspect of the invention is a method of treating a subject having cancer, comprising administering a therapeutically effective amount of the isolated bispecific EGFR/c-Met FN3 domain containing molecule, the EGFR binding FN3 domain, the c-Met binding FN3 domain or the bispecific EGFR/c-Met antibody of the invention to a patient in need thereof for a time sufficient to treat the cancer.

The bispecific EGFR/c-Met FN3 domain containing molecule, the EGFR binding FN3 domain, the c-Met binding FN3 domain or the bispecific EGFR/c-Met antibodies of the invention may be used for treatment of any disease or disorder characterized by abnormal activation or production of EGFR, c-Met, EGF, soluble EGFR, soluble c-Met or other EGFR ligand or HGF, or disorder related to EGFR or c-Met expression, which may or may not involve malignancy or cancer, where abnormal activation and/or production of EGFR, c-Met, EGF or other EGFR ligand, or HGF is occurring in cells or tissues of a subject having, or predisposed to, the disease or disorder.

The FN3 domains that specifically bind c-Met and block binding of HGF to c-Met of the invention may be for treatment of tumors, including cancers and benign tumors. Cancers that are amenable to treatment by the c-Met binding FN3 domains of the invention include those that overexpress c-Met. Exemplary cancers that are amenable to treatment by the FN3 domains of the invention include epithelial cell cancers, breast cancer, ovarian cancer, lung cancer, colorectal cancer, anal cancer, prostate cancer, kidney cancer, bladder cancer, head and neck cancer, gastric cancer, ovarian cancer, pancreatic cancer, skin cancer, oral cancer, esophageal cancer, vaginal cancer, cervical cancer, cancer of the spleen, testicular cancer, and cancer of the thymus.

The FN3 domains that specifically bind EGFR and blocks binding of EGF to the EGFR of the invention may be used for treatment of tumors, including cancers and benign tumors. Cancers that are amenable to treatment by the FN3 domains of the invention include those that overexpress EGFR or variants. Exemplary cancers that are amenable to treatment by the FN3 domains of the invention include epithelial cell cancers, breast cancer, ovarian cancer, lung cancer, colorectal cancer, anal cancer, prostate cancer, kidney cancer, bladder cancer, head and neck cancer, ovarian cancer, pancreatic cancer, skin cancer, oral cancer, esophageal cancer, vaginal cancer, cervical cancer, cancer of the spleen, testicular cancer, and cancer of the thymus. The bispecific EGFR/c-Met FN3 domain containing molecules or the bispecific EGFR/c-Met antibodies of the invention may be used for treatment of tumors, including cancers and benign tumors. Exemplary cancers that are amenable to treatment by the bispecific EGFR/c-Met FN3 domain containing molecule or the bispecific EGFR/c-Met antibody of the invention include those that over-express EGFR and/or c-Met, cancers associated with elevated EGFR activity and/or expression levels (such as, for example, an EGFR activating mutation, an EGFR gene amplification, or ligand mediated EGFR activation) and elevated c-Met activity and/or expression levels (such as, for example, a c-Met activating mutation, a c-Met gene amplification, or HGF mediated c-Met activation).

Exemplary EGFR activating mutations that may be associated with cancer include point mutations, deletion mutations, insertion mutations, inversions or gene amplifications that lead to an increase in at least one biological activity of EGFR, such as elevated tyrosine kinase activity, formation of receptor homodimers and heterodimers, enhanced ligand binding etc. Mutations can be located in any portion of an EGFR gene or regulatory region associated with an EGFR gene and include mutations in exon 18, 19, 20 or 21 or mutations in the kinase domain. Exemplary activating EGFR mutations are G719A, L861X (X being any amino acid), L858R, E746K, L747S, E749Q, A750P, A755V, V765M, L858P or T790M substitutions, deletion of E746-A750, deletion of R748-P753, insertion of Ala between M766 and A767, insertion of SVA (Ser, Val, Ala) between S768 and V769, and insertion of NS (Asn, Ser) between P772 and H773. Other examples of EGFR activating mutations are known in the art (see e.g., U.S. Pat. Publ. No. US2005/0272083). Information about EGFR and other ErbB receptors including receptor homo- and hetero-dimers, receptor ligands, autophosphorylation sites, and signaling molecules involved in ErbB mediated signaling is known in the art (see e.g., Hynes and Lane, Nature Reviews Cancer 5: 341-354, 2005).

Exemplary c-Met activating mutations include point mutations, deletion mutations, insertion mutations, inversions or gene amplifications that lead to an increase in at least one biological activity of a c-Met protein, such as elevated tyrosine kinase activity, formation of receptor homodimers and heterodimers, enhanced ligand binding etc. Mutations can be located in any portion of the c-Met gene or regulatory regions associated with the gene, such as mutations in the kinase domain of c-Met. Exemplary c-Met activating mutations are mutations at residue positions N375, V13, V923, R175, V136, L229, S323, R988, S1058/T1010 and E168. Methods for detecting EGFR and c-Met mutations or gene amplifications are well known.

Exemplary cancers that are amenable to treatment by the bispecific molecules of the invention such as the bispecific EGFR/c-Met antibodies of the invention include epithelial cell cancers, breast cancer, ovarian cancer, lung cancer, non-small cell lung cancer (NSCLC), lung adenocarcinoma, small cell lung cancer, colorectal cancer, anal cancer, prostate cancer, kidney cancer, bladder cancer, head and neck cancer, pharynx cancer, cancer of the nose, pancreatic cancer, skin cancer, oral cancer, cancer of the tongue, esophageal cancer, vaginal cancer, cervical cancer, cancer of the spleen, testicular cancer, gastric cancer, cancer of the thymus, colon cancer, thyroid cancer, liver cancer (hepatocellular carcinoma (HCC)) or sporadic or hereditary papillary renal cell carcinoma (PRCC).

Another aspect of the invention is a method of treating a subject having cancer, comprising administering a therapeutically effective amount of the isolated bispecific EGFR/c-Met antibody of the invention to a patient in need thereof for a time sufficient to treat the cancer, wherein the subject is homozygous for phenylalanine at position 158 of CD16 (FcγRIIIa-158F/F genotype) or heterozygous for valine and phenylalanine at position 158 of CD16 (FcγRIIIa-158F/V genotype). CD16 is also known as the Fc gamma receptor IIIa (FcγRIIIa) or the low affinity immunoglobulin gamma Fc region receptor III-A isoform. Valine/phenylalanine (V/F) polymorphism at FcγRIIIa protein residue position 158 has been shown to affect FcγRIIIa affinity to human IgG. Receptor with FcγRIIa-158F/F or FcγRIIa-158F/V polymorphisms demonstrates reduced Fc engagement and therefore reduced ADCC when compared to the FcγRIIIa-158V/V. The lack of or low amount of fucose on human N-linked oligosaccharides improves the ability of the antibodies to induce ADCC due to improved binding of the antibodies to human FcγRIIIa (CD16)(Shields et al., J Biol Chem 277:26733-40, 2002). The antibodies of the invention have reduced fucose content of about between 1% to about 10%. In some embodiments, the bispecific EGFR/c-Met antibody has a glycan structure with fucose content of about 50%, 40%, 45%, 40%, 35%, 30%, 25%, 20%, 15%, 10%, 9%, 8%, 7%, 6%, 5%, 4%, 3%, 2% or 1%. Therefore, the antibodies of the invention may be more efficacious in the treatment of patients with FcγRIIIa-158F/F or FcγRIIIa-158F/V genotypes. Patients can be analyzed for their FcγRIIa polymorphism using routine methods.

In some methods described herein, the antibodies of the invention may be used to treat a subject having cancer that is resistant or has acquired resistance to treatment with one or more EGFR inhibitors. Exemplary EGFR inhibitors for which cancer may acquire resistance are anti-EGFR antibodies cetuximab (Erbitux®), panitumumab (Vectibix), matuzumab, nimotuzumab, small molecule EGFR inhibitors Tarceva® (erlotinib), IRESSA (gefitinib), EKB-569 (pelitinib, irreversible EGFR TKI), pan-ErbB and other receptor tyrosine kinase inhibitors, lapatinib (EGFR and HER2 inhibitor), pelitinib (EGFR and HER2 inhibitor), vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BTBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR TKI), and HKI-272 (neratinib, irreversible EGFR/ErbB2 inhibitor). The methods described herein may be used to treat cancer that is resistant to treatment with gefitinib, erlotinib, afatinib, CO-1686, AZD9291 and/or cetuximab. An exemplary antibody that can be used is EM1-mAb.

Another aspect of the invention is a method of treating a subject having cancer, comprising administering a therapeutically effective amount of the isolated bispecific EGFR/c-Met antibody of the invention to a patient in need thereof for a time sufficient to treat the cancer, wherein the subject is resistant or has acquired resistance to treatment with erlotinib, gefitinib, afatinib, CO-1686, AZD9291 or cetuximab.

Various qualitative and/or quantitative methods may be used to determine if a subject is resistant, has developed or is susceptible to developing a resistance to treatment with an EGFR inhibitor. Symptoms that may be associated with resistance to an EGFR inhibitor include, for example, a decline or plateau of the well-being of the patient, an increase in the size of a tumor, arrested or slowed decline in growth of a tumor, and/or the spread of cancerous cells in the body from one location to other organs, tissues or cells. Re-establishment or worsening of various symptoms associated with cancer may also be an indication that a subject has developed or is susceptible to developing resistance to EGFR inhibitors, such as anorexia, cognitive dysfunction, depression, dyspnea, fatigue, hormonal disturbances, neutropenia, pain, peripheral neuropathy, and sexual dysfunction. The symptoms associated with cancer may vary according to the type of cancer. For example, symptoms associated with cervical cancer may include abnormal bleeding, unusual heavy vaginal discharge, pelvic pain that is not related to the normal menstrual cycle, bladder pain or pain during urination, and bleeding between regular menstrual periods, after sexual intercourse, douching, or pelvic exam. Symptoms associated with lung cancer may include persistent cough, coughing up blood, shortness of breath, wheezing chest pain, loss of appetite, losing weight without trying and fatigue. Symptoms for liver cancer may include loss of appetite and weight, abdominal pain, especially in the upper right part of abdomen that may extend into the back and shoulder, nausea and vomiting, general weakness and fatigue, an enlarged liver, abdominal swelling (ascites), and a yellow discoloration of the skin and the whites of eyes (jaundice). One skilled in oncology may readily identify symptoms associated with a particular cancer type.

Others means to determine if a subject has developed a resistance to an EGFR inhibitor include examining EGFR phosphorylation, ERK1/2 phosphorylation and/or AKT phosphorylation in cancer cells, where increased phosphorylation may be indicative that the subject has developed or is susceptible to developing resistance to an EGFR inhibitor. Methods of determining EGFR, ERK1/2 and/or AKT phosphorylation are well known and described herein. Identification of a subject who has developed a resistance to an EGFR inhibitor may involve detection of elevated c-Met expression levels or elevated c-Met activity, for example, arising from increased levels of circulating HGF, an activating mutation of the c-Met gene or a c-Met gene amplification.

Another embodiment of the invention is a method of treating NSCLC in a patient having an NSCLC tumor or tumor metastasis having an activating EGFR mutation or EGFR gene amplification, comprising administering to the patient a therapeutically effective amount of the bispecific EGFR/c-Met antibody of the invention.

The bispecific EGFR/c-Met antibodies of the invention can be used to treat non-small cell lung cancer (NSCLC), which includes squamous cell carcinoma, adenocarcinoma, and large cell carcinoma. In some embodiments, cells of the NSCLC have an epithelial phenotype. In some embodiments, the NSCLC has acquired resistance to treatment with one or more EGFR inhibitors.

In NSCLC, specific mutations in the EGFR gene are associated with high response rates (70-80%) to EGFR tyrosine kinase inhibitors (EGFR-TKIs). A 5 amino acid deletion in exon 19 or the point mutation L858R in EGFR are associated with EGFR-TKI sensitivity (Nakata and Gotoh, Expert Opin Ther Targets 16:771-781, 2012). These mutations result in a ligand-independent activation of the EGFR kinase activity. Activating EGFR mutations occur in 10-30% of NSCLC patients and are significantly more common in East Asians, women, never smokers, and patients with adenocarcinoma histology (Janne and Johnson Clin Cancer Res 12(14 Suppl): 4416s-4420s, 2006). EGFR gene amplification is also strongly correlated with response after EGFR-TKI treatment (Cappuzzo et al., J Natl Cancer Inst 97:643-55, 2005).

Although the majority of NSCLC patients with EGFR mutations initially respond to EGFR TKI therapy, virtually all acquire resistance that prevents a durable response. 50-60% of patients acquire resistance due to a second-site point mutation in the kinase domain of EGFR (T790M). Nearly 60% of all tumors that become resistant to EGFR tyrosine kinase inhibitors increase c-Met expression, amplify the c-Met gene, or increase its only known ligand, HGF (Turke et al., Cancer Cell, 17:77-88, 2010).

Another embodiments of the invention is a method of treating patient having cancer, comprising administering a therapeutically effective amount of the bispecific EGFR/c-Met antibody of the invention to a patient in need thereof for a time sufficient to treat the cancer, wherein the cancer is associated with an EGFR activating mutation, an EGFR gene amplification, increased levels of circulating HGF, a c-Met activating mutation, a c-Met gene amplification or a mutant KRAS.

In some embodiments the EGFR activating mutation is G719A, G719X (X being any amino acid), L861X (X being any amino acid), L858R, E746K, L747S, E749Q, A750P, A755V, V765M, L858P or T790M substitution, deletion of E746-A750, deletion of R748-P753, insertion of Ala (A) between M766 and A767, insertion of Ser, Val and Ala (SVA) between S768 and V769, and insertion of Asn and Ser (NS) between P772 and H773.

Another embodiments of the invention is a method of treating patient having cancer, comprising administering a therapeutically effective amount of the bispecific EGFR/c-Met antibody of the invention to a patient in need thereof for a time sufficient to treat the cancer, wherein the cancer is associated with an EGFR mutation L858R, T790M or deletion of residues E746-A750 (del(E746, A750)), EGFR amplification or c-Met amplification.

In some embodiments, the cancer is associated with wild type EGFR and wild type c-Met.

In some embodiments, the cancer is associated with wild type EGFR and c-Met amplification.

In some embodiments, the cancer is associated with EGFR L858R and T790M mutations and wild type c-Met.

In some embodiments, the cancer is associated with EGFR deletion del (E764, A750) and wild type c-Met.

In some embodiments, the cancer is associated with EGFR deletion del(E764, A750) and c-Met amplification.

In some embodiments, the cancer is associated with EGFR deletion del(E764, A750), EGFR amplification and c-Met amplification.

In some embodiments, the patient has a NSCLC associated with EGFR L858R and T790M mutations and wild type c-Met.

In some embodiments, the patient has a NSCLC associated with EGFR amplification and wild type c-Met.

In some embodiments, the patient has a NSCLC associated with EGFR amplification and c-Met amplification.

In some embodiments, the patient has a NSCLC associated with EGFR deletion del(E764, A750) and wild type c-Met.

In some embodiments, the patient has a NSCLC associated with EGFR deletion del(E764, A750) and c-Met amplification.

In some embodiments, the patients are treated with the EM1-mAb of the invention. The EM1-mAb of the invention shows efficacy in In vivo tumor animal models, when the tumors are associated with L858R, T790M, del(E746, A750) EGFR, EGFR amplification, wild type c-Met and/or c-Met amplification. Amplification of EGFR or c-Met may be evaluated by standard methods, for example by determining the copy number of the EGFR or c-Met gene by southern blotting, FISH, or comparative genomic hybridization (CGH).

Another embodiments of the invention is a method of treating patient having cancer, comprising administering a therapeutically effective amount of the bispecific EGFR/c-Met antibody of the invention to a patient in need thereof for a time sufficient to treat the cancer, wherein the cancer is associated with EGFR mutations L858R, T790M or deletion of residues E746-A750 (del(E746, A750)), EGFR amplification or c-Met amplification, and mutant KRAS.

In some embodiments, the mutant KRAS has a G12V substitution. KRAS belongs to the family of RAS proto-oncogenes encoding guanosine triphosphatases (GTPases), and mediates EGFR signal transduction downstream of the receptor. Tumors with proto-oncogenic KRAS mutations such as the activating G12V or G12C mutation would therefore not be expected to be treatable by EGFR antibodies. Clinical studies with anti-EGFR antibodies cetuximab or panitumumab demonstrated that patients with KRAS-mutated colorectal tumors do not respond to these agents (Van Cutsem et al., N Eng J Med 360:1408-1417, 2009; Lievr et al., J Clin Oncol 26:374-379, 2008; Amado et al., J Clin Oncol 26:1626-1634m 2008). The bispecific EGFR/c-Met antibodies of the invention mediate KRAS mutant cell line killing via effective ADCC, and therefore, contrary to the current anti-EGFR therapies, may be efficacious in treatment of patients whose cancer is associated with KRAS activating mutations. Such exemplary antibody is the EM1-mAb.

The terms “treat” or “treatment” refers to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount of the bispecific EGFR/c-Met antibody of the invention may vary according to factors such as the disease state, age, sex, and weight of the individual, and the ability of the bispecific EGFR/c-Met antibody of the invention to elicit a desired response in the individual. Exemplary indicators of an effective EGFR/c-Met therapeutic that may decline or abate in association with resistance include, for example, improved well-being of the patient, decrease or shrinkage of the size of a tumor, arrested or slowed growth of a tumor, and/or absence of metastasis of cancer cells to other locations in the body.

### Administration/Pharmaceutical Compositions

The invention provides for pharmaceutical compositions comprising the bispecific EGFR/c-Met antibody of the invention and a pharmaceutically acceptable carrier. For therapeutic use, the bispecific EGFR/c-Met FN3 domain containing molecules, the EGFR-binding FN3 domains, the c-Met-binding FN3 domains or the bispecific EGFR/c-Met antibodies of the invention may be prepared as pharmaceutical compositions containing an effective amount of the domain, molecule or antibody as an active ingredient in a pharmaceutically acceptable carrier. The term “carrier” refers to a diluent, adjuvant, excipient, or vehicle with which the active compound is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules or antibodies of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition. Troy, D. B. ed., Lipincott Williams and Wilkins, Philadelphia, PA 2006, Part 5, Pharmaceutical Manufacturing pp 691-1092, See especially pp. 958-989.

The mode of administration for therapeutic use of the bispecific EGFR/c-Met FN3 domain containing molecules, the EGFR-binding FN3 domains, the c-Met-binding FN3 domains or the bispecific EGFR/c-Met antibodies of the invention may be any suitable route that delivers the agent to the host, such as parenteral administration, e.g., intradermal, intramuscular, intraperitoneal intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Thus, a pharmaceutical composition of the invention for intramuscular injection may be prepared to contain 1 ml sterile buffered water, and between about 1 ng to about 100 mg/kg, e.g. about 50 ng to about 30 mg/kg or more preferably, about 5 mg to about 25 mg/kg, of the bispecific EGFR/c-Met FN3 domain containing molecules, the EGFR-binding FN3 domains or the c-Met-binding FN3 domains of the invention.

The bispecific EGFR/c-Met antibodies of the invention may be administered to a patient by any suitable route, for example parentally by intravenous (IV) infusion or bolus injection, intramuscularly or subcutaneously or intraperitoneally. IV infusion can be given over as little as 15 minutes, but more often for 30 minutes, 60 minutes, 90 minutes or even 2 or 3 hours. The bispecific EGFR/c-Met antibodies of the invention may also be injected directly into the site of disease (e.g., the tumor itself). The dose given to a patient having a cancer is sufficient to alleviate or at least partially arrest the disease being treated (“therapeutically effective amount”) and may be sometimes 0.1 to 10 mg/kg body weight, for example 1, 2, 3, 4, 5, 6, 7, 8, 9 or 10 mg/kg, but may even higher, for example 15, 20, 30, 40, 50, 60, 70, 80, 90 or 100 mg/kg. A fixed unit dose may also be given, for example, 50, 100, 200, 500 or 1000 mg, or the dose may be based on the patient's surface area, e.g., 400, 300, 250, 200, or 100 mg/m². Usually between 1 and 8 doses, (e.g., 1, 2, 3, 4, 5, 6, 7 or 8) may be administered to treat cancer, but 10, 12, 20 or more doses may be given. Administration of the bispecific EGFR/c-Met antibody of the invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

For example, a pharmaceutical composition comprising the bispecific EGFR/c-Met antibody of the invention for intravenous infusion may be made up to contain about 200 ml of sterile Ringer's solution, and about 8 mg to about 2400 mg, about 400 mg to about 1600 mg, or about 400 mg to about 800 mg of the bispecific EGFR/c-Met antibody for administration to a 80 kg patient. Methods for preparing parenterally administrable compositions are well known and are described in more detail in, for example, “Remington's Pharmaceutical Science”, 15th ed., Mack Publishing Company, Easton, PA.

The bispecific EGFR/c-Met FN3 domain containing molecules, the EGFR-binding FN3 domains, the c-Met-binding FN3 domains or the bispecific EGFR/c-Met antibodies of the invention may be lyophilized for storage and reconstituted in a suitable carrier prior to use. This technique has been shown to be effective with conventional protein preparations and well known lyophilization and reconstitution techniques can be employed.

The bispecific EGFR/c-Met FN3 domain containing molecules, the EGFR-binding FN3 domains, the c-Met-binding FN3 domains or the bispecific EGFR/c-Met antibodies of the invention may be administered in combination with a second therapeutic agent simultaneously, sequentially or separately. The second therapeutic agent may be a chemotherapeutic agent or a targeted anti-cancer therapy.

The bispecific EGFR/c-Met antibody may be administered together with any one or more of the chemotherapeutic drugs or other anti-cancer therapeutics known to those of skill in the art. Chemotherapeutic agents are chemical compounds useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL® docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbinc; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT®), toceranib (PALLADIA®), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA®), BIBW 2992 (TOVOK™), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et al., eds.), Chapter 10, McMillan Publishing, are also applicable to the methods of the present invention.

Exemplary agents that may be used in combination with the bispecific EGFR/c-Met FN3 domain containing molecules, the EGFR-binding FN3 domains, the c-Met-binding FN3 domains or the bispecific EGFR/c-Met antibodies of the invention include tyrosine kinase inhibitors and targeted anti-cancer therapies such as Iressa® (gefitinib) and Tarceva (erlotinib) and other antagonists of HER2, HER3, HER4 or VEGF. Exemplary HER2 antagonists include CP-724-714, HERCEPTIN® (trastuzumab), OMNITARG™ (pertuzumab), TAK-165, lapatinib (EGFR and HER2 inhibitor), and GW-282974. Exemplary HER3 antagonists include anti-Her3 antibodies (see e.g., U.S. Pat. Publ. No. US2004/0197332). Exemplary HER4 antagonists include anti-HER4 siRNAs (see e.g., Maatta et al., Mol Biol Cell 17: 67-79, 2006. An exemplary VEGF antagonist is Bevacizumab (Avastin™).

When a small molecule is used in combination with the bispecific EGFR/c-Met antibody of the invention, it is typically administered more often, preferably once a day, but 2, 3, 4 or more times per day is also possible, as is every two days, weekly or at some other interval. Small molecule drugs are often taken orally but parenteral administration is also possible, e.g., by IV infusion or bolus injection or subcutaneously or intramuscularly. Doses of small molecule drugs may typically be from 10 to 1000 mg, or about 100, 150, 200 or 250 mg.

When the bispecific EGFR/c-Met antibody of the invention is administered in combination with a second therapeutic agent, the combination may take place over any convenient timeframe. For example, the bispecific EGFR/c-Met antibody and the second therapeutic agent may be administered to a patient on the same day, and even in the same intravenous infusion. However, the bispecific EGFR/c-Met antibody and the second therapeutic agent may also be administered on alternating days or alternating weeks, fortnights or months, and so on. In some methods, the bispecific EGFR/c-Met antibody and the second therapeutic agent are administered with sufficient proximity in time that they are simultaneously present (e.g., in the serum) at detectable levels in the patient being treated. In some methods, an entire course of treatment of the bispecific EGFR/c-Met antibody consisting of a number of doses over a time period is followed or preceded by a course of treatment of the second therapeutic agent also consisting of a number of doses. In some methods, treatment with the bispecific EGFR/c-Met antibody administered second is begun if the patient has resistance or develops resistance to the second therapeutic agent administered initially. The patient may receive only a single course or multiple courses of treatment with one or both the bispecific EGFR/c-Met antibody and the second therapeutic agent. A recovery period of 1, 2 or several days or weeks may be used between administration of the bispecific EGFR/c-Met antibody and the second therapeutic agent. When a suitable treatment regimen has already been established for the second therapeutic agent, that regimen may be used in combination with the bispecific EGFR/c-Met antibody of the invention. For example, Tarceva® (erlotinib) is taken as a 100 mg or 150 mg pill once a day, and Iressa® (gefitinib) is taken as 250 mg tablet daily.

The bispecific EGFR/c-Met antibody, optionally in combination with the second therapeutic agent may be administered together with any form of radiation therapy

What is claimed:

1. An isolated bispecific epidermal growth factor receptor (EGFR)/hepatocyte growth factor receptor (c-Met) antibody, comprising:
a) a first heavy chain (HC1) comprising a HC1 constant domain 3 (HC1 CH3) and a HC1 variable region 1 (VH1);
b) a first light chain (LC1) comprising a light chain variable region 1 (VL1);
c) a second heavy chain (HC2) comprising a HC2 constant domain 3 (HC2 CH3) and a HC2 variable region 2 (VH2); and
d) a second light chain (LC2) comprising a light chain variable region 2 (VL2), wherein the VH1 comprises a heavy chain complementarity determining region 1 (HCDR1), a HCDR2 and a HCDR3 amino acid sequences of SEQ ID NOs: 210, 211 and 212, respectively; the VL1 comprises a light chain complementarity determining region 1 (LCDR1), a LCDR2 and a LCDR3 amino acid sequences of SEQ ID NOs: 213, 214 and 215, respectively, the VH2 comprises the HCDR1, the HCDR2 and the HCDR3 amino acid sequences of SEQ ID NOs: 216,217 and 218, respectively; and the VL2 comprises the LCDR1, the LCDR2 and the LCDR3 amino acid sequences of SEQ ID NOs: 219, 220 and 221, respectively.

2. The bispecific antibody of claim 1, wherein the antibody inhibits growth of NCI-H292 or NCI-H1975 cells with an IC₅₀ value that is at least about 300-fold less, at least about 400-fold less, at least about 500-fold less, at least about 600-fold less, at least about 700-fold less or at least about 800-fold less when compared to the IC₅₀ value of inhibition of growth of NCI-H292 or NCI-H1975 cells with cetuximab when NCI-H292 or NCI-H1975 cells are grown in low attachment conditions.

3. The bispecific antibody of claim 1, wherein the antibody inhibits growth of HGF-expressing SKMES-1 cell tumor in SCID Beige mice with a percentage (%) T/C value of at least 500-fold less on day 36 when compared to cetuximab, when the bispecific antibody and cetuximab are administered at 20 mg/kg dose.

4. The bispecific antibody of claim 1, wherein the VH1, the VL1, the VH2 and the VL2 comprise the amino acid sequences of SEQ ID NOs: 189, 190, 193 and 194, respectively.

5. The bispecific antibody of claim 4, wherein the HC1 and the HC2 are an IgG1 isotype.

6. The bispecific antibody of claim 5, wherein the HC1 CH3 comprises at least one substitution and the HC2 CH3 comprises at least one substitution at residue positions 405 or 409.

7. The bispecific antibody of claim 6, wherein the HC1 CH3 comprises a K409R or a F405L substitution and the HC2 CH3 comprises a K409R or a F405L substitution.

8. The bispecific antibody of claim 7, wherein
a) the HC1 CH3 comprises the F405L substitution and the HC2 CH3 comprises the K409R substitution; or
b) the HC1 CH3 comprises the K409R substitution and the HC2 CH3 comprises the F405L substitution.

9. The bispecific antibody of claim 1, wherein the HC1 comprises the amino acid sequence of SEQ ID NO: 204 and the HC2 comprises the amino acid sequence of SEQ ID NO: 203.

10. The bispecific antibody of claim 9, wherein the antibody has a biantennary glycan structure with a fucose content of about between 1% to about 15%.

11. A method of treating a subject having cancer, comprising administering a therapeutically effective amount of the bispecific EGFR/c-Met antibody of claim 1 to a patient in need thereof to treat the cancer.

12. The method of claim 11, wherein the cancer is associated with an EGFR activating mutation, an EGFR gene amplification, increased levels of circulating HGF, a c-Met activating mutation, a c-Met gene amplification or a mutant KRAS.

13. The method of claim 12, wherein the EGFR activating mutation is G719A, G719X (X being any amino acid), L861X (X being any amino acid), L858R, E746K, L747S, E749Q, A750P, A755V, V765M, L858P or T790M substitution, deletion of E746-A750, deletion of R748-P753, insertion of Ala (A) between M766 and A767, insertion of Ser, Val and Ala (SVA) between S768 and V769, and insertion of Asn and Ser (NS) between P772 and H773.

14. The method of claim 12, wherein the mutant KRAS has a G12V or G12C substitution.

15. The method of claim 11, wherein the subject is resistant or has acquired resistance to treatment with erlotinib, gefitinib, afatinib, CO-1686, AZD9192 or cetuximab.

16. The method of claim 11, wherein the cancer is an epithelial cell cancer, breast cancer, ovarian cancer, lung cancer, non-small cell lung cancer (NSCLC), lung adenocarcinoma, small cell lung cancer, colorectal cancer, anal cancer, prostate cancer, kidney cancer, bladder cancer, head and neck cancer, pharynx cancer, cancer of the nose, pancreatic cancer, skin cancer, oral cancer, cancer of the tongue, esophageal cancer, vaginal cancer, cervical cancer, cancer of the spleen, testicular cancer, gastric cancer, cancer of the thymus, colon cancer, thyroid cancer, liver cancer, hepatocellular carcinoma (HCC) or sporadic or hereditary papillary renal cell carcinoma (PRCC).

17. The method of claim 11, wherein the subject is homozygous for phenylalanine at position 158 of CD16 or heterozygous for valine and phenylalanine at position 158 of CD16.

18. The method of claim 11, comprising administering a second therapeutic agent.

19. The method of claim 18, wherein the second therapeutic agent is a chemotherapeutic agent or a targeted anti-cancer therapy.

20. The method of claim 11, wherein the second therapeutic agent is cisplatin, vinblastine or a tyrosine kinase inhibitor of EGFR, c-Met, HER2, HER3, HER4 or VEGFR.

21. A method of inhibiting growth or proliferation of cells that express EGFR and/or c-Met, comprising contacting the cells with the bispecific antibody of claim 1.

22. A method of inhibiting growth or metastasis of EGFR and/or c-Met expressing tumor or cancer cells in a subject comprising administering to the subject an effective amount of the bispecific antibody of claim 1 to inhibit the growth or metastasis of EGFR and/or c-Met expressing tumor or cancer cells.

23. The method of claim 22, wherein the EGFR and/or c-Met expressing tumor is an epithelial cell cancer, breast cancer, ovarian cancer, lung cancer, non-small cell lung cancer (NSCLC), lung adenocarcinoma, small cell lung cancer, colorectal cancer, anal cancer, prostate cancer, kidney cancer, bladder cancer, head and neck cancer, pharynx cancer, cancer of the nose, pancreatic cancer, skin cancer, oral cancer, cancer of the tongue, esophageal cancer, vaginal cancer, cervical cancer, cancer of the spleen, testicular cancer, gastric cancer, cancer of the thymus, colon cancer, thyroid cancer, liver cancer, hepatocellular carcinoma (HCC) or sporadic or hereditary papillary renal cell carcinoma (PRCC).

24. The method of claim 23, wherein the EGFR and/or c-Met expressing tumor is associated with an EGFR activating mutation, an EGFR gene amplification, increased levels of circulating HGF, a c-Met activating mutation, a c-Met gene amplification or a mutant KRAS.

25. The method of claim 24, wherein the EGFR activating mutation is G719A, G719X (X being any amino acid), L861X (X being any amino acid), L858R, E746K, L747S, E749Q, A750P, A755V, V765M, L858P or T790M substitution, deletion of E746-A750, deletion of R748-P753, insertion of Ala (A) between M766 and A767, insertion of Ser, Val and Ala (SVA) between S768 and V769, and insertion of Asn and Ser (NS) between P772 and H773.

26. The method of claim 24, wherein the mutant KRAS has a G12V or G12C substitution.


SEQUENCE LISTING (240 sequences)

SEQ ID NO: 1 - protein, 89 aa, synthetic construct
  REGION 1..89: note = Tencon FN3 scaffold
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 2 - DNA, 58 bp, synthetic construct
  misc_feature 1..58: note = primer
  source 1..58: mol_type = other DNA
ggaaacaggatctaccatgctgccggcgccgaaaaacctggttgtttctgaagttacc

SEQ ID NO: 3 - DNA, 21 bp, synthetic construct
  misc_feature 1..21: note = primer
  source 1..21: mol_type = other DNA
aacaccgtagatagaaacggt

SEQ ID NO: 4 - DNA, 131 bp, synthetic construct
  misc_feature 1..131: note = primer
  source 1..131: mol_type = other DNA
cggcggttagaacgcggctacaattaatacataaccccatccccctgttgacaattaatcatcggctcgtataatgtgtggaattgtgagcggataacaatttcacacaggaaacaggatctaccatgctg

SEQ ID NO: 5 - DNA, 21 bp, synthetic construct
  misc_feature 1..21: note = primer
  source 1..21: mol_type = other DNA
cggcggttagaacgcggctac

SEQ ID NO: 6 - DNA, 66 bp, synthetic construct
  misc_feature 1..66: note = primer
  misc_difference 26..27: note = n is a, c, g, or t
  misc_difference 29..30: note = n is a, c, g, or t
  misc_difference 32..33: note = n is a, c, g, or t
  misc_difference 35..36: note = n is a, c, g, or t
  misc_difference 38..39: note = n is a, c, g, or t
  misc_difference 41..42: note = n is a, c, g, or t
  misc_difference 44..45: note = n is a, c, g, or t
  source 1..66: mol_type = other DNA
ggtggtgaattccgcagacagcggsnnsnnsnnsnnsnnsnnsnnaacaccgtagatagaaacggt

SEQ ID NO: 7 - DNA, 69 bp, synthetic construct
  misc_feature 1..69: note = primer
  misc_difference 26..27: note = n is a, c, g, or t
  misc_difference 29..30: note = n is a, c, g, or t
  misc_difference 32..33: note = n is a, c, g, or t
  misc_difference 35..36: note = n is a, c, g, or t
  misc_difference 38..39: note = n is a, c, g, or t
  misc_difference 41..42: note = n is a, c, g, or t
  misc_difference 44..45: note = n is a, c, g, or t
  misc_difference 47..48: note = n is a, c, g, or t
  source 1..69: mol_type = other DNA
ggtggtgaattccgcagacagcggsnnsnnsnnsnnsnnsnnsnnsnnaacaccgtagatagaaacggt

SEQ ID NO: 8 - DNA, 72 bp, synthetic construct
  misc_feature 1..72: note = primer
  misc_difference 26..27: note = n is a, c, g, or t
  misc_difference 29..30: note = n is a, c, g, or t
  misc_difference 32..33: note = n is a, c, g, or t
  misc_difference 35..36: note = n is a, c, g, or t
  misc_difference 38..39: note = n is a, c, g, or t
  misc_difference 41..42: note = n is a, c, g, or t
  misc_difference 44..45: note = n is a, c, g, or t
  misc_difference 47..48: note = n is a, c, g, or t
  misc_difference 50..51: note = n is a, c, g, or t
  source 1..72: mol_type = other DNA
ggtggtgaattccgcagacagcggsnnsnnsnnsnnsnnsnnsnnsnnsnnaacaccgtagatagaaacggt

SEQ ID NO: 9 - DNA, 75 bp, synthetic construct
  misc_feature 1..75: note = primer
  misc_difference 26..27: note = n is a, c, g, or t
  misc_difference 29..30: note = n is a, c, g, or t
  misc_difference 32..33: note = n is a, c, g, or t
  misc_difference 35..36: note = n is a, c, g, or t
  misc_difference 38..39: note = n is a, c, g, or t
  misc_difference 41..42: note = n is a, c, g, or t
  misc_difference 44..45: note = n is a, c, g, or t
  misc_difference 47..48: note = n is a, c, g, or t
  misc_difference 50..51: note = n is a, c, g, or t
  misc_difference 53..54: note = n is a, c, g, or t
  source 1..75: mol_type = other DNA
ggtggtgaattccgcagacagcggsnnsnnsnnsnnsnnsnnsnnsnnsnnsnnaacaccgtagatagaaacggt

SEQ ID NO: 10 - DNA, 80 bp, synthetic construct
  misc_feature 1..80: note = primer
  misc_difference 28..29: note = n is a, c, g, or t
  misc_difference 31..32: note = n is a, c, g, or t
  misc_difference 34..35: note = n is a, c, g, or t
  misc_difference 37..38: note = n is a, c, g, or t
  misc_difference 40..41: note = n is a, c, g, or t
  misc_difference 43..44: note = n is a, c, g, or t
  misc_difference 46..47: note = n is a, c, g, or t
  misc_difference 49..50: note = n is a, c, g, or t
  misc_difference 52..53: note = n is a, c, g, or t
  misc_difference 55..56: note = n is a, c, g, or t
  misc_difference 58..59: note = n is a, c, g, or t
  source 1..80: mol_type = other DNA
rmggtggtgaattccgcagacagcggsnnsnnsnnsnnsnnsnnsnnsnnsnnsnnsnnaacaccgtagatagaaacggt

SEQ ID NO: 11 - DNA, 81 bp, synthetic construct
  misc_feature 1..81: note = primer
  misc_difference 26..27: note = n is a, c, g, or t
  misc_difference 29..30: note = n is a, c, g, or t
  misc_difference 32..33: note = n is a, c, g, or t
  misc_difference 35..36: note = n is a, c, g, or t
  misc_difference 38..39: note = n is a, c, g, or t
  misc_difference 41..42: note = n is a, c, g, or t
  misc_difference 44..45: note = n is a, c, g, or t
  misc_difference 47..48: note = n is a, c, g, or t
  misc_difference 50..51: note = n is a, c, g, or t
  misc_difference 53..54: note = n is a, c, g, or t
  misc_difference 56..57: note = n is a, c, g, or t
  misc_difference 59..60: note = n is a, c, g, or t
  source 1..81: mol_type = other DNA
ggtggtgaattccgcagacagcggsnnsnnsnnsnnsnnsnnsnnsnnsnnsnnsnnsnnaacaccgtagatagaaacggt

SEQ ID NO: 12 - DNA, 81 bp, synthetic construct
  misc_feature 1..81: note = primer
  source 1..81: mol_type = other DNA
aagatcagttgcggccgctagactagaaccgctgccatggtgatggtgatggtgaccgccggtggtgaattccgcagacag

SEQ ID NO: 13 - DNA, 30 bp, synthetic construct
  misc_feature 1..30: note = primer
  source 1..30: mol_type = other DNA
cggcggttagaacgcggctacaattaatac

SEQ ID NO: 14 - DNA, 22 bp, synthetic construct
  misc_feature 1..22: note = primer
  source 1..22: mol_type = other DNA
catgattacgccaagctcagaa

SEQ ID NO: 15 - DNA, 65 bp, synthetic construct
  misc_feature 1..65: note = primer
  source 1..65: mol_type = other DNA
gagccgccgccaccggtttaatggtgatggtgatggtgaccaccggtggtgaattccgcagacag

SEQ ID NO: 16 - DNA, 37 bp, synthetic construct
  misc_feature 1..37: note = primer
  source 1..37: mol_type = other DNA
aagaaggagaaccggtatgctgccggcgccgaaaaac

SEQ ID NO: 17 - DNA, 89 bp, synthetic construct
  misc_feature 1..89: note = primer
  source 1..89: mol_type = other DNA
tttgggaagcttctaggtctcggcggtcaccatcaccatcaccatggcagcggttctagtctagcggccccaactgatcttcaccaaac

SEQ ID NO: 18 - protein, 94 aa, synthetic construct
  REGION 1..94: note = EGFR binding FN3 domain
LPAPKNLVVSEVTEDSLRLSWADPHGFYDSFLIQYQESEKVGEAINLTVPGSERSYDLTGLKPGTEYTVSIYGVHNVYKDTNMRGLPLSAEFTT

SEQ ID NO: 19 - protein, 94 aa, synthetic construct
  REGION 1..94: note = EGFR binding FN3 domain
LPAPKNLVVSEVTEDSLRLSWTYDRDGYDSFLIQYQESEKVGEAINLTVPGSERSYDLTGLKPGTEYTVSIYGVHNVYKDTNMRGLPLSAEFTT

SEQ ID NO: 20 - protein, 94 aa, synthetic construct
  REGION 1..94: note = EGFR binding FN3 domain
LPAPKNLVVSEVTEDSLRLSWGYNGDHFDSFLIQYQESEKVGEAINLTVPGSERSYDLTGLKPGTEYTVSIYGVHNVYKDTNMRGLPLSAEFTT

SEQ ID NO: 21 - protein, 94 aa, synthetic construct
  REGION 1..94: note = EGFR binding FN3 domain
LPAPKNLVVSEVTEDSLRLSWDDPRGFYESFLIQYQESEKVGEAINLTVPGSERSYDLTGLKPGTEYTVSIYGVHNVYKDTNMRGLPLSAEFTT

SEQ ID NO: 22 - protein, 94 aa, synthetic construct
  REGION 1..94: note = EGFR binding FN3 domain
LPAPKNLVVSEVTEDSLRLSWTWPYADLDSFLIQYQESEKVGEAINLTVPGSERSYDLTGLKPGTEYTVSIYGVHNVYKDTNMRGLPLSAEFTT

SEQ ID NO: 23 - protein, 94 aa, synthetic construct; identical to SEQ ID NO: 20
  REGION 1..94: note = EGFR binding FN3 domain

SEQ ID NO: 24 - protein, 95 aa, synthetic construct
  REGION 1..95: note = EGFR binding FN3 domain
LPAPKNLVVSEVTEDSLRLSWDYDLGVYFDSFLIQYQESEKVGEAINLTVPGSERSYDLTGLKPGTEYTVSIYGVHNVYKDTNMRGLPLSAEFTT

SEQ ID NO: 25 - protein, 94 aa, synthetic construct
  REGION 1..94: note = EGFR binding FN3 domain
LPAPKNLVVSEVTEDSLRLSWDDPWAFYESFLIQYQESEKVGEAINLTVPGSERSYDLTGLKPGTEYTVSIYGVHNVYKDTNMRGLPLSAEFTT

SEQ ID NO: 26 - protein, 94 aa, synthetic construct
  REGION 1..94: note = EGFR binding FN3 domain
LPAPKNLVVSEVTEDSLRLSWTAPDAAFDSFLIQYQESEKVGEAINLTVPGSERSYDLTGLKPGTEYTVSIYGVLGSYVFEHDVMLPLSAEFTT

SEQ ID NO: 27 - protein, 94 aa, synthetic construct
  REGION 1..94: note = EGFR binding FN3 domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTT

SEQ ID NO: 28 - protein, 94 aa, synthetic construct
  REGION 1..94: note = EGFR binding FN3 domain
LPAPKNLVVSEVTEDSARLSWTAPDAAFDSFLIQYQESEKVGEAIVLTVPGSERSYDLTGLKPGTEYTVSIYGVLGSYVFEHDVMLPLSAIFTT

SEQ ID NO: 29 - protein, 94 aa, synthetic construct; identical to SEQ ID NO: 22
  REGION 1..94: note = EGFR binding FN3 domain

SEQ ID NO: 30 - protein, 37 aa, synthetic construct
  REGION 1..37: note = primer
AAGAAGGAGAACCGGTATGCTGCCGGCGCCGAAAAAC

SEQ ID NO: 31 - protein, 65 aa, synthetic construct
  REGION 1..65: note = primer
GAGCCGCCGCCACCGGTTTAATGGTGATGGTGATGGTGACCACCGGTGGTGAAGATCGCAGACAG

SEQ ID NO: 32 - protein, 89 aa, synthetic construct
  REGION 1..89: note = c-Met binding FN3 domain
LPAPKNLVVSRVTEDSARLSWTAPDAAFDSFWIRYDEVVVGGEAIVLTVPGSERSYDLTGLKPGTEYYVNILGVKGGSISVPLSAIFTT

SEQ ID NO: 33 - protein, 89 aa, synthetic construct
  REGION 1..89: note = c-Met binding FN3 domain
LPAPKNLVVSRVTEDSARLSWTAPDAAFDSFFIRYDEFLRSGEAIVLTVPGSERSYDLTGLKPGTEYWVTILGVKGGLVSTPLSAIFTT

SEQ ID NO: 34 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYIVNIMGVKGGSISHPLSAIFTT

SEQ ID NO: 35 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVNILGVKGGGLSVPLSAIFTT

SEQ ID NO: 36 - protein, 89 aa, synthetic construct
  REGION 1..89: note = c-Met binding FN3 domain
LPAPKNLVVSRVTEDSARLSWTAPDAAFDSFVIRYFEFLGSGEAIVLTVPGSERSYDLTGLKPGTEYVVQILGVKGGYISIPLSAIFTT

SEQ ID NO: 37 - protein, 89 aa, synthetic construct
  REGION 1..89: note = c-Met binding FN3 domain
LPAPKNLVVSRVTEDSARLSWTAPDAAFDSFWIRYLEFLLGGEAIVLTVPGSERSYDLTGLKPGTEYVVQIMGVKGGTVSPPLSAIFTT

SEQ ID NO: 38 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVGINGVKGGYISYPLSAIFTT

SEQ ID NO: 39 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTDLKPGTEYGVTINGVKGGRVSTPLSAIFTT

SEQ ID NO: 40 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVQIIGVKGGHISLPLSAIFTT

SEQ ID NO: 41 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 42 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYAVNIMGVKGGRVSVPLSAIFTT

SEQ ID NO: 43 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVQILGVKGGSISVPLSAIFTT

SEQ ID NO: 44 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVNIMGVKGGSISYPLSAIFTT

SEQ ID NO: 45 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVQILGVKGGYISIPLSAIFTT

SEQ ID NO: 46 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVQIMGVKGGTVSPPLSAIFTT

SEQ ID NO: 47 - protein, 89 aa, synthetic construct
  REGION 1..89: note = c-Met binding FN3 domain
LPAPKNLVVSRVTEDSARLSWTAPDAAFDSFWIRYFEFTTAGEAIVLTVPGSERSYDLTGLKPGTEYVVNIMGVKGGSISPPLSAIFTT

SEQ ID NO: 48 - protein, 89 aa, synthetic construct
  REGION 1..89: note = c-Met binding FN3 domain
LPAPKNLVVSRVTEDSARLSWTAPDAAFDSFWIRYFELLSTGEAIVLTVPGSERSYDLTGLKPGTEYVVNIMGVKGGSISPPLSAIFTT

SEQ ID NO: 49 - protein, 89 aa, synthetic construct
  REGION 1..89: note = c-Met binding FN3 domain
LPAPKNLVVSRVTEDSARLSWTAPDAAFDSFWIRYFEFVSKGEAIVLTVPGSERSYDLTGLKPGTEYVVNIMGVKGGSISPPLSAIFTT

SEQ ID NO: 50 - protein, 205 aa, synthetic construct
  REGION 1..205: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFWIRYDEVVVGGEAIVLTVPGSERSYDLTGLKPGTEYYVNILGVKGGSISVPLSAIFTT

SEQ ID NO: 51 - protein, 204 aa, synthetic construct
  REGION 1..204: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGGGSGGGGSGGGGSGGGGSLPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 52 - protein, 205 aa, synthetic construct
  REGION 1..205: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFWIRYFEFLGSGEAIVLTVPGSERSYDLTGLKPGTEYVVQIIGVKGGHISLPLSAIFTT

SEQ ID NO: 53 - protein, 205 aa, synthetic construct
  REGION 1..205: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFFIRYDEFLRSGEAIVLTVPGSERSYDLTGLKPGTEYWVTILGVKGGLVSTPLSAIFTT

SEQ ID NO: 54 - protein, 205 aa, synthetic construct
  REGION 1..205: note = bispecific EGFR/cMet binding molecule
MLPAPKNLVVSEVTEDSARLSWADPHGFYDSFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 55 - protein, 205 aa, synthetic construct
  REGION 1..205: note = bispecific EGFR/cMet binding molecule
MLPAPKNLVVSEVTEDSARLSWADPHGFYDSFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFWIRYFEFLGSGEAIVLTVPGSERSYDLTGLKPGTEYVVQIIGVKGGHISLPLSAIFTT

SEQ ID NO: 56 - protein, 205 aa, synthetic construct; identical to SEQ ID NO: 55
  REGION 1..205: note = bispecific EGFR/cMet binding molecule

SEQ ID NO: 57 - protein, 194 aa, synthetic construct
  REGION 1..194: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 58 - protein, 194 aa, synthetic construct
  REGION 1..194: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYDEVVVGGEAIVLTVPGSERSYDLTGLKPGTEYYVNILGVKGGSISVPLSAIFTT

SEQ ID NO: 59 - protein, 195 aa, synthetic construct
  REGION 1..195: note = bispecific EGFR/cMet binding molecule
MLPAPKNLVVSEVTEDSARLSWADPHGFYDSFLIQYQESEKVGEAIVLTVPGSERSYDLTGLKPGTEYTVSIYGVHNVYKDTNMRGLPLSAIFTTAPAPAPAPAPMLPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 60 - protein, 194 aa, synthetic construct
  REGION 1..194: note = bispecific EGFR/cMet binding molecule
MLPAPKNLVVSEVTEDSARLSWADPHGFYDSFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYDEVVVGGEAIVLTVPGSERSYDLTGLKPGTEYYVNILGVKGGSISVPLSAIFTT

SEQ ID NO: 61 - protein, 194 aa, synthetic construct
  REGION 1..194: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILGVKGGKISPPLSAIFTT

SEQ ID NO: 62 - protein, 194 aa, synthetic construct
  REGION 1..194: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVGSGEAIVLTVPGSERSYDLTGLKPGTEYVVNILGVKGGSISPPLSAIFTT

SEQ ID NO: 63 - protein, 194 aa, synthetic construct
  REGION 1..194: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVSKGDAIVLTVPGSERSYDLTGLKPGTEYVVNILGVKGGSISPPLSAIFTT

SEQ ID NO: 64 - protein, 194 aa, synthetic construct
  REGION 1..194: note = bispecific EGFR/cMet binding molecule
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILSVKGGSISPPLSAIFTT

SEQ ID NO: 65 - protein, 194 aa, synthetic construct
  REGION 1..194: note = bispecific EGFR/cMet binding molecule
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILGVKGGKISPPLSAIFTT

SEQ ID NO: 66 - protein, 194 aa, synthetic construct
  REGION 1..194: note = bispecific EGFR/cMet binding molecule
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVGSGEAIVLTVPGSERSYDLTGLKPGTEYVVNILGVKGGSISPPLSAIFTT

SEQ ID NO: 67 - protein, 194 aa, synthetic construct
  REGION 1..194: note = bispecific EGFR/cMet binding molecule
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVSKGDAIVLTVPGSERSYDLTGLKPGTEYVVNILGVKGGSISPPLSAIFTT

SEQ ID NO: 68 - protein, 194 aa, synthetic construct
  REGION 1..194: note = bispecific EGFR/cMet binding molecule
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILSVKGGSISPPLSAIFTT

SEQ ID NO: 69 - protein, 194 aa, synthetic construct
  REGION 1..194: note = bispecific EGFR/cMet binding molecule
MLPAPKNLVVSEVTEDSARLSWADPHGFYDSFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILGVKGGKISPPLSAIFTT

SEQ ID NO: 70 - protein, 194 aa, synthetic construct
  REGION 1..194: note = bispecific EGFR/cMet binding molecule
MLPAPKNLVVSEVTEDSARLSWADPHGFYDSFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVGSGEAIVLTVPGSERSYDLTGLKPGTEYVVNILGVKGGSISPPLSAIFTT

SEQ ID NO: 71 - protein, 194 aa, synthetic construct
  REGION 1..194: note = bispecific EGFR/cMet binding molecule
MLPAPKNLVVSEVTEDSARLSWADPHGFYDSFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVSKGDAIVLTVPGSERSYDLTGLKPGTEYVVNILGVKGGSISPPLSAIFTT

SEQ ID NO: 72 - protein, 194 aa, synthetic construct
  REGION 1..194: note = bispecific EGFR/cMet binding molecule
MLPAPKNLVVSEVTEDSARLSWADPHGFYDSFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILSVKGGSISPPLSAIFTT

SEQ ID NO: 73 - protein, 1210 aa, Homo sapiens [sequence omitted]

SEQ ID NO: 74 - protein, 53 aa, Homo sapiens
NSDSECPLSHDGYCLHDGVCMYIEALDKYACNCVVGYIGERCQYRDLKWWELR

SEQ ID NO: 75 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 76 - protein, 89 aa, synthetic construct
  REGION 1..89: note = Fibcon FN3 domain
LDAPTDLQVTNVTDTSITVSWTPPSATITGYRITYTPSNGPGEPKELTVPPSSTSVTITGLTPGVEYVVSLYALKDNQESPPLVGTQTT

SEQ ID NO: 77 - protein, 94 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 78 - protein, 4 aa, synthetic construct
  REGION 1..4: note = linker
GSGS

SEQ ID NO: 79 - protein, 25 aa, synthetic construct
  REGION 1..25: note = linker
GGGGSGGGGSGGGGSGGGGSGGGGS

SEQ ID NO: 80 - protein, 4 aa, synthetic construct
  REGION 1..4: note = linker
APAP

SEQ ID NO: 81 - protein, 10 aa, synthetic construct
  REGION 1..10: note = linker
APAPAPAPAP

SEQ ID NO: 82 - protein, 20 aa, synthetic construct
  REGION 1..20: note = linker
APAPAPAPAPAPAPAPAPAP

SEQ ID NO: 83 - protein, 40 aa, synthetic construct
  REGION 1..40: note = linker
APAPAPAPAPAPAPAPAPAPAPAPAPAPAPAPAPAPAPAP

SEQ ID NO: 84 - protein, 29 aa, synthetic construct
  REGION 1..29: note = linker
AEAAAKEAAAKEAAAKEAAAKEAAAKAAA

SEQ ID NO: 85 - protein, 8 aa, synthetic construct
  REGION 1..8: note = tencon BC loop
TAPDAAFD

SEQ ID NO: 86 - protein, 7 aa, synthetic construct
  REGION 1..7: note = tencon FG loop
KGGHRSN

SEQ ID NO: 87 - protein, 8 aa, synthetic construct
  REGION 1..8: note = EGFR binding FN3 domain BC loop
ADPHGFYD

SEQ ID NO: 88 - protein, 8 aa, synthetic construct
  REGION 1..8: note = EGFR binding FN3 domain BC loop
TYDRDGYD

SEQ ID NO: 89 - protein, 8 aa, synthetic construct
  REGION 1..8: note = EGFR binding FN3 domain BC loop
WDPFSFYD

SEQ ID NO: 90 - protein, 8 aa, synthetic construct
  REGION 1..8: note = EGFR binding FN3 domain BC loop
DDPRGFYE

SEQ ID NO: 91 - protein, 8 aa, synthetic construct
  REGION 1..8: note = EGFR binding FN3 domain BC loop
TWPYADLD

SEQ ID NO: 92 - protein, 8 aa, synthetic construct
  REGION 1..8: note = EGFR binding FN3 domain BC loop
GYNGDHFD

SEQ ID NO: 93 - protein, 8 aa, synthetic construct
  REGION 1..8: note = EGFR binding FN3 domain BC loop
DYDLGVYD

SEQ ID NO: 94 - protein, 8 aa, synthetic construct
  REGION 1..8: note = EGFR binding FN3 domain BC loop
DDPWDFYE

SEQ ID NO: 95 - protein, 12 aa, synthetic construct
  REGION 1..12: note = EGFR binding FN3 domain FG loop
HNVYKDTNMRGL

SEQ ID NO: 96 - protein, 11 aa, synthetic construct
  REGION 1..11: note = EGFR binding FN3 domain FG loop
LGSYVFEHDVM

SEQ ID NO: 97 - DNA, 285 bp, synthetic construct
  misc_feature 1..285: note = EGFR binding FN3 domain from ECB97; P54AR4-83V22
  source 1..285: mol_type = other DNA
atgttgccagcgccgaagaacctggtagttagcgaggttactgaggacagcgcgcgtctgagctgggacgatccgtgggcgttctacgagagctttctgatccagtatcaagagagcgagaaagtcggtgaagcgattgtgctgaccgtcccgggctccgagcgttcctacgacctgaccggtttgaagccgggtaccgagtatacggtgagcatctacggtgttcacaatgtctataaggacactaatatccgcggtctgcctctgagcgccattttcaccacc

SEQ ID NO: 98 - DNA, 285 bp, synthetic construct
  misc_feature 1..285: note = EGFR binding FN3 domain from ECB15; P54AR4-83V2
  source 1..285: mol_type = other DNA
atgctgccagcccctaagaatctggtcgtgagcgaagtaaccgaggacagcgcccgcctgagctgggacgacccgtgggcgttctatgagtctttcctgattcagtatcaagaaagcgaaaaagttggcgaagcgatcgtcctgaccgtcccgggtagcgagcgctcctacgatctgaccggcctgaaaccgggtacggagtacacggtgtccatttacggtgttcacaatgtgtataaagacaccaacatgcgtggcctgccgctgtcggcgattttcaccacc

SEQ ID NO: 99 - protein, 89 aa, synthetic construct
  REGION 1..89: note = tencon27 FN3 domain
LPAPKNLVVSRVTEDSARLSWTAPDAAFDSFLIQYQESEKVGEAIVLTVPGSERSYDLTGLKPGTEYTVSIYGVKGGHRSNPLSAIFTT

SEQ ID NO: 100 - protein, 89 aa, synthetic construct
  REGION 1..89: note = TCL14 library
  VARIANT 32: note = MISC_FEATURE - Xaa is any amino acid
  VARIANT 34: note = MISC_FEATURE - Xaa is any amino acid
  VARIANT 36: note = MISC_FEATURE - Xaa is any amino acid
  VARIANT 38: note = MISC_FEATURE - Xaa is any amino acid
  VARIANT 39: note = MISC_FEATURE - Xaa is any amino acid
  VARIANT 40: note = MISC_FEATURE - Xaa is any amino acid
  VARIANT 41: note = MISC_FEATURE - Xaa is any amino acid
  VARIANT 68: note = MISC_FEATURE - Xaa is any amino acid
  VARIANT 70: note = MISC_FEATURE - Xaa is any amino acid
  VARIANT 72: note = MISC_FEATURE - Xaa is any amino acid
  VARIANT 78: note = MISC_FEATURE - Xaa is any amino acid
  VARIANT 79: note = MISC_FEATURE - Xaa is any amino acid
  VARIANT 81: note = MISC_FEATURE - Xaa is any amino acid
LPAPKNLVVSRVTEDSARLSWTAPDAAFDSFXIXYXEXXXXGEAIVLTVPGSERSYDLTGLKPGTEYXVXIXGVKGGXXSXPLSAIFTT

SEQ ID NO: 101 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 102 - protein, 697 aa, Homo sapiens
QRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 103 - DNA, 276 bp, synthetic construct
  misc_feature 1..276: note = c-Met binding FN3 domain
  source 1..276: mol_type = other DNA
ctgccggctccgaagaacttggtggtgagccgtgttaccgaagatagcgcacgcctgagctggacggcaccggatgcggcgttcgatagcttctggattcgctattttgagtttctgggtagcggtgaggcaattgttctgacggtgccgggctctgaacgctcctacgatttgaccggtctgaaaccgggcaccgagtatgtggtgaacattctgagcgttaagggcggtagcatcagcccaccgctgagcgcgatcttcacgactggtggttgc

SEQ ID NO: 104 - DNA, 267 bp, synthetic construct
  misc_feature 1..267: note = c-Met binding FN3 domain
  source 1..267: mol_type = other DNA
ctgccggcaccgaagaacctggttgtcagccgtgtgaccgaggatagcgcacgtttgagctggaccgctccggatgcagcctttgacagcttctggattcgttactttgaatttctgggtagcggtgaggcgatcgttctgacggtgccgggctctgaacgcagctatgatttgacgggcctgaagccgggtactgagtacgtggttaacatcatgggcgttaagggtggtaaaatcagcccgccattgtccgcgatctttaccacg

SEQ ID NO: 105 - protein, 5 aa, synthetic construct
  REGION 1..5: note = linker
GGGGS

SEQ ID NO: 106 - protein, 194 aa, synthetic construct; identical to SEQ ID NO: 64
  REGION 1..194: note = Bispecific EGFR/c-Met binding molecule

SEQ ID NO: 107 - protein, 94 aa, synthetic construct
  REGION 1..94: note = EGFR binding FN3 domain
LPAPKNLVVSEVTEDSARLSWADPHGFYDSFLIQYQESEKVGEAIVLTVPGSERSYDLTGLKPGTEYTVSIYGVHNVYKDTNMRGLPLSAIFTT

SEQ ID NO: 108 - protein, 94 aa, synthetic construct
  REGION 1..94: note = EGFR binding FN3 domain
LPAPKNLVVSEVTEDSARLSWDDPWAFYESFLIQYQESEKVGEAIVLTVPGSERSYDLTGLKPGTEYTVSIYGVHNVYKDTNIRGLPLSAIFTT

SEQ ID NO: 109 - protein, 94 aa, synthetic construct
  REGION 1..94: note = EGFR binding FN3 domain
LPAPKNLVVSEVTEDSARLSWDDPHAFYESFLIQYQESEKVGEAIVLTVPGSERSYDLTGLKPGTEYTVSIYGVHNVYKDTNIRGLPLSAIFTT

SEQ ID NO: 110 - protein, 94 aa, synthetic construct
  REGION 1..94: note = EGFR binding FN3 domain
LPAPKNLVVSEVTEDSARLSWADPHGFYDSFLIQYQESEKVGEAIVLTVPGSERSYDLTGLKPGTEYTVSIYGVHNVYKDTNIRGLPLSAIFTT

SEQ ID NO: 111 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVNILGVKGGKISPPLSAIFTT

SEQ ID NO: 112 - protein, 89 aa, synthetic construct
  REGION 1..89: note = c-Met binding FN3 domain
LPAPKNLVVSRVTEDSARLSWTAPDAAFDSFWIRYFEFVGSGEAIVLTVPGSERSYDLTGLKPGTEYVVNILGVKGGSISPPLSAIFTT

SEQ ID NO: 113 - protein, 89 aa, synthetic construct
  REGION 1..89: note = c-Met binding FN3 domain
LPAPKNLVVSRVTEDSARLSWTAPDAAFDSFWIRYFEFVSKGDAIVLTVPGSERSYDLTGLKPGTEYVVNILGVKGGSISPPLSAIFTT

SEQ ID NO: 114 - protein, 89 aa, synthetic construct
  REGION 1..89: note = c-Met binding FN3 domain
LPAPKNLVVSRVTEDSARLSWTAPDAAFDSFWIRYFEFLGSGEAIVLTVPGSERSYDLTGLKPGTEYVVNILSVKGGSISPPLSAIFTT

SEQ ID NO: 115 - DNA, 591 bp, synthetic construct
  misc_feature 1..591: note = Bispecific EGFR/c-Met binding molecule
  source 1..591: mol_type = other DNA
atgttgccagcgccgaagaacctggtagttagcgaggttactgaggacagcgcgcgtctgagctgggacgatccgtgggcgttctacgagagctttctgatccagtatcaagagagcgagaaagtcggtgaagcgattgtgctgaccgtcccgggctccgagcgttcctacgacctgaccggtttgaagccgggtaccgagtatacggtgagcatctacggtgttcacaatgtctataaggacactaatatccgcggtctgcctctgagcgccattttcaccaccgcaccggcaccggctccggctcctgccccgctgccggctccgaagaacttggtggtgagccgtgttaccgaagatagcgcacgcctgagctggacggcaccggatgcggcgttcgatagcttctggattcgctattttgagtttctgggtagcggtgaggcaattgttctgacggtgccgggctctgaacgctcctacgatttgaccggtctgaaaccgggcaccgagtatgtggtgaacattctgagcgttaagggcggtagcatcagcccaccgctgagcgcgatcttcacgactggtggttgc

SEQ ID NO: 116 - DNA, 582 bp, synthetic construct
  misc_feature 1..582: note = Bispecific EGFR/c-Met binding molecule
  source 1..582: mol_type = other DNA
atgctgccagcccctaagaatctggtcgtgagcgaagtaaccgaggacagcgcccgcctgagctgggacgacccgtgggcgttctatgagtctttcctgattcagtatcaagaaagcgaaaaagttggcgaagcgatcgtcctgaccgtcccgggtagcgagcgctcctacgatctgaccggcctgaaaccgggtacggagtacacggtgtccatttacggtgttcacaatgtgtataaagacaccaacatgcgtggcctgccgctgtcggcgattttcaccaccgcgcctgcgccagcgcctgcaccggctccgctgccggcaccgaagaacctggttgtcagccgtgtgaccgaggatagcgcacgtttgagctggaccgctccggatgcagcctttgacagcttctggattcgttactttgaatttctgggtagcggtgaggcgatcgttctgacggtgccgggctctgaacgcagctatgatttgacgggcctgaagccgggtactgagtacgtggttaacatcatgggcgttaagggtggtaaaatcagcccgccattgtccgcgatctttaccacg

SEQ ID NO: 117 - protein, 51 aa, synthetic construct
  REGION 1..51: note = albumin binding domain
TIDEWLLKEAKEKAIEELKKAGITSDYYFDLINKAKTVEGVNALKDEILKA

SEQ ID NO: 118 - protein, 250 aa, synthetic construct
  REGION 1..250: note = Bispecific EGFR/c-Met binding molecule
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYDEVVVGGEAIVLTVPGSERSYDLTGLKPGTEYYVNILGVKGGSISVPLSAIFTTAPAPAPAPAPLAEAKVLANRELDKYGVSDYYKNLINNAKTVEGVKALLDEILAALP

SEQ ID NO: 119 - protein, 250 aa, synthetic construct
  REGION 1..250: note = Bispecific EGFR/c-Met binding molecule
MLPAPKNLVVSEVTEDSARLSWADPHGFYDSFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYDEVVVGGEAIVLTVPGSERSYDLTGLKPGTEYYVNILGVKGGSISVPLSAIFTTAPAPAPAPAPLAEAKVLANRELDKYGVSDYYKNLINNAKTVEGVKALLDEILAALP

SEQ ID NO: 120 - protein, 250 aa, synthetic construct
  REGION 1..250: note = Bispecific EGFR/c-Met binding molecule
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYFEFLGSGEAIVLTVPGSERSYDLTGLKPGTEYVVNIMGVKGGKISPPLSAIFTTAPAPAPAPAPLAEAKVLANRELDKYGVSDYYKNLINNAKTVEGVKALLDEILAALP

SEQ ID NO: 121 - protein, 250 aa, synthetic construct
  REGION 1..250: note = Bispecific EGFR/c-Met binding molecule
MLPAPKNLVVSEVTEDSARLSWADPHGFYDSFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYFEFLGSGEAIVLTVPGSERSYDLTGLKPGTEYVVNIMGVKGGKISPPLSAIFTTAPAPAPAPAPLAEAKVLANRELDKYGVSDYYKNLINNAKTVEGVKALLDEILAALP

SEQ ID NO: 122 - protein, 95 aa, synthetic construct
  REGION 1..95: note = EGFR binding FN3 domain
MLPAPKNLVVSEVTEDSLRLSWADPHGFYDSFLIQYQESEKVGEAINLTVPGSERSYDLTGLKPGTEYTVSIYGVHNVYKDTNMRGLPLSAEFTT

SEQ ID NO: 123 - protein, 95 aa, synthetic construct
  REGION 1..95: note = EGFR binding FN3 domain
MLPAPKNLVVSEVTEDSLRLSWTYDRDGYDSFLIQYQESEKVGEAINLTVPGSERSYDLTGLKPGTEYTVSIYGVHNVYKDTNMRGLPLSAEFTT

SEQ ID NO: 124 - protein, 95 aa, synthetic construct
  REGION 1..95: note = EGFR binding FN3 domain
MLPAPKNLVVSEVTEDSLRLSWGYNGDHFDSFLIQYQESEKVGEAINLTVPGSERSYDLTGLKPGTEYTVSIYGVHNVYKDTNMRGLPLSAEFTT

SEQ ID NO: 125 - protein, 95 aa, synthetic construct
  REGION 1..95: note = EGFR binding FN3 domain
MLPAPKNLVVSEVTEDSLRLSWDDPRGFYESFLIQYQESEKVGEAINLTVPGSERSYDLTGLKPGTEYTVSIYGVHNVYKDTNMRGLPLSAEFTT

SEQ ID NO: 126 - protein, 95 aa, synthetic construct
  REGION 1..95: note = EGFR binding FN3 domain
MLPAPKNLVVSEVTEDSLRLSWTWPYADLDSFLIQYQESEKVGEAINLTVPGSERSYDLTGLKPGTEYTVSIYGVHNVYKDTNMRGLPLSAEFTT

SEQ ID NO: 127 - protein, 95 aa, synthetic construct; identical to SEQ ID NO: 124
  REGION 1..95: note = EGFR binding FN3 domain

SEQ ID NO: 128 - protein, 96 aa, synthetic construct
  REGION 1..96: note = EGFR binding FN3 domain
MLPAPKNLVVSEVTEDSLRLSWDYDLGVYFDSFLIQYQESEKVGEAINLTVPGSERSYDLTGLKPGTEYTVSIYGVHNVYKDTNMRGLPLSAEFTT

SEQ ID NO: 129 - protein, 95 aa, synthetic construct
  REGION 1..95: note = EGFR binding FN3 domain
MLPAPKNLVVSEVTEDSLRLSWDDPWAFYESFLIQYQESEKVGEAINLTVPGSERSYDLTGLKPGTEYTVSIYGVHNVYKDTNMRGLPLSAEFTT

SEQ ID NO: 130 - protein, 95 aa, synthetic construct
  REGION 1..95: note = EGFR binding FN3 domain
MLPAPKNLVVSEVTEDSLRLSWTAPDAAFDSFLIQYQESEKVGEAINLTVPGSERSYDLTGLKPGTEYTVSIYGVLGSYVFEHDVMLPLSAEFTT

SEQ ID NO: 131 - protein, 95 aa, synthetic construct
  REGION 1..95: note = EGFR binding FN3 domain
MLPAPKNLVVSEVTEDSARLSWDDPWAFYESFLIQYQESEKVGEAIVLTVPGSERSYDLTGLKPGTEYTVSIYGVHNVYKDTNMRGLPLSAIFTT

SEQ ID NO: 132 - protein, 95 aa, synthetic construct
  REGION 1..95: note = EGFR binding FN3 domain
MLPAPKNLVVSEVTEDSARLSWTAPDAAFDSFLIQYQESEKVGEAIVLTVPGSERSYDLTGLKPGTEYTVSIYGVLGSYVFEHDVMLPLSAIFTT

SEQ ID NO: 133 - protein, 95 aa, synthetic construct; identical to SEQ ID NO: 126
  REGION 1..95: note = EGFR binding FN3 domain

SEQ ID NO: 134 - protein, 95 aa, synthetic construct
  REGION 1..95: note = EGFR binding FN3 domain
MLPAPKNLVVSEVTEDSARLSWADPHGFYDSFLIQYQESEKVGEAIVLTVPGSERSYDLTGLKPGTEYTVSIYGVHNVYKDTNMRGLPLSAIFTT

SEQ ID NO: 135 - protein, 95 aa, synthetic construct
  REGION 1..95: note = EGFR binding FN3 domain
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTT

SEQ ID NO: 136 - protein, 95 aa, synthetic construct
  REGION 1..95: note = EGFR binding FN3 domain
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTT

SEQ ID NO: 137 - protein, 95 aa, synthetic construct
  REGION 1..95: note = EGFR binding FN3 domain
MLPAPKNLVVSEVTEDSARLSWADPHGFYDSFLIQYQESEKVGEAIVLTVPGSERSYDLTGLKPGTEYTVSIYGVHNVYKDTNIRGLPLSAIFTT

SEQ ID NO: 138 - protein, 204 aa, synthetic construct
  REGION 1..204: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFWIRYDEVVVGGEAIVLTVPGSERSYDLTGLKPGTEYYVNILGVKGGSISVPLSAIFTT

SEQ ID NO: 139 - protein, 203 aa, synthetic construct
  REGION 1..203: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGGGSGGGGSGGGGSGGGGSLPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 140 - protein, 204 aa, synthetic construct
  REGION 1..204: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFWIRYFEFLGSGEAIVLTVPGSERSYDLTGLKPGTEYVVQIIGVKGGHISLPLSAIFTT

SEQ ID NO: 141 - protein, 204 aa, synthetic construct
  REGION 1..204: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFFIRYDEFLRSGEAIVLTVPGSERSYDLTGLKPGTEYWVTILGVKGGLVSTPLSAIFTT

SEQ ID NO: 142 - protein, 204 aa, synthetic construct
  REGION 1..204: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 143 - protein, 204 aa, synthetic construct
  REGION 1..204: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNMRGLPLSAIFTTGGGGSGGGGSGGGGSGGGGSMLPAPKNLVVSRVTEDSARLSWTAPDAAFDSFWIRYFEFLGSGEAIVLTVPGSERSYDLTGLKPGTEYVVQIIGVKGGHISLPLSAIFTT

SEQ ID NO: 144 - protein, 204 aa, synthetic construct; identical to SEQ ID NO: 143
  REGION 1..204: note = Bispecific EGFR/c-Met binding domain

SEQ ID NO: 145 - protein, 193 aa, synthetic construct
  REGION 1..193: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 146 - protein, 193 aa, synthetic construct
  REGION 1..193: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYDEVVVGGEAIVLTVPGSERSYDLTGLKPGTEYYVNILGVKGGSISVPLSAIFTT

SEQ ID NO: 147 - protein, 194 aa, synthetic construct
  REGION 1..194: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNMRGLPLSAIFTTAPAPAPAPAPMLPAPKNLVVSRVTEDSARLSWTAPDAAFDSFWIRYFEFLGSGEAIVLTVPGSERSYDLTGLKPGTEYVVNIMGVKGGKISPPLSAIFTT

SEQ ID NO: 148 - protein, 193 aa, synthetic construct
  REGION 1..193: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYDEVVVGGEAIVLTVPGSERSYDLTGLKPGTEYYVNILGVKGGSISVPLSAIFTT

SEQ ID NO: 149 - protein, 193 aa, synthetic construct
  REGION 1..193: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILGVKGGKISPPLSAIFTT

SEQ ID NO: 150 - protein, 193 aa, synthetic construct
  REGION 1..193: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVGSGEAIVLTVPGSERSYDLTGLKPGTEYVVNILGVKGGSISPPLSAIFTT

SEQ ID NO: 151 - protein, 193 aa, synthetic construct
  REGION 1..193: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVSKGDAIVLTVPGSERSYDLTGLKPGTEYVVNILGVKGGSISPPLSAIFTT

SEQ ID NO: 152 - protein, 193 aa, synthetic construct
  REGION 1..193: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILSVKGGSISPPLSAIFTT

SEQ ID NO: 153 - protein, 193 aa, synthetic construct
  REGION 1..193: note = Bispecific EGFR/c-Met binding domain
LPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILGVKGGKISPPLSAIFTT

SEQ ID NO: 154 - protein, 193 aa, synthetic construct
  REGION 1..193: note = Bispecific EGFR/c-Met binding domain
LPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVGSGEAIVLTVPGSERSYDLTGLKPGTEYVVNILGVKGGSISPPLSAIFTT

SEQ ID NO: 155 - protein, 193 aa, synthetic construct
  REGION 1..193: note = Bispecific EGFR/c-Met binding domain
LPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVSKGDAIVLTVPGSERSYDLTGLKPGTEYVVNILGVKGGSISPPLSAIFTT

SEQ ID NO: 156 - protein, 193 aa, synthetic construct
  REGION 1..193: note = Bispecific EGFR/c-Met binding domain
LPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILSVKGGSISPPLSAIFTT

SEQ ID NO: 157 - protein, 193 aa, synthetic construct
  REGION 1..193: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILGVKGGKISPPLSAIFTT

SEQ ID NO: 158 - protein, 193 aa, synthetic construct
  REGION 1..193: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVGSGEAIVLTVPGSERSYDLTGLKPGTEYVVNILGVKGGSISPPLSAIFTT

SEQ ID NO: 159 - protein, 193 aa, synthetic construct
  REGION 1..193: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVSKGDAIVLTVPGSERSYDLTGLKPGTEYVVNILGVKGGSISPPLSAIFTT

SEQ ID NO: 160 - protein, 193 aa, synthetic construct
  REGION 1..193: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILSVKGGSISPPLSAIFTT

SEQ ID NO: 161 - protein, 193 aa, synthetic construct; identical to SEQ ID NO: 152
  REGION 1..193: note = Bispecific EGFR/c-Met binding domain

SEQ ID NO: 162 - protein, 249 aa, synthetic construct
  REGION 1..249: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYDEVVVGGEAIVLTVPGSERSYDLTGLKPGTEYYVNILGVKGGSISVPLSAIFTTAPAPAPAPAPLAEAKVLANRELDKYGVSDYYKNLINNAKTVEGVKALLDEILAALP

SEQ ID NO: 163 - protein, 249 aa, synthetic construct
  REGION 1..249: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYDEVVVGGEAIVLTVPGSERSYDLTGLKPGTEYYVNILGVKGGSISVPLSAIFTTAPAPAPAPAPLAEAKVLANRELDKYGVSDYYKNLINNAKTVEGVKALLDEILAALP

SEQ ID NO: 164 - protein, 249 aa, synthetic construct
  REGION 1..249: note = Bispecific EGFR/c-Met binding domain
LPAPKNLVVSEVTEDSARLSWDDPWAFYESFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYFEFLGSGEAIVLTVPGSERSYDLTGLKPGTEYVVNIMGVKGGKISPPLSAIFTTAPAPAPAPAPLAEAKVLANRELDKYGVSDYYKNLINNAKTVEGVKALLDEILAALP

SEQ ID NO: 165 - protein, 249 aa, synthetic construct
  REGION 1..249: note = Bispecific EGFR/c-Met binding domain
LPAPKNLVVSEVTEDSARLSWADPHGFYDSFLIQYQESEKVGEAIVLTVPGSERSYDLTGLKPGTEYTVSIYGVHNVYKDTNMRGLPLSAIFTTAPAPAPAPAPLPAPKNLVVSRVTEDSARLSWTAPDAAFDSFWIRYFEFLGSGEAIVLTVPGSERSYDLTGLKPGTEYVVNIMGVKGGKISPPLSAIFTTAPAPAPAPAPLAEAKVLANRELDKYGVSDYYKNLINNAKTVEGVKALLDEILAALP

SEQ ID NO: 166 - DNA, 588 bp, synthetic construct
  misc_feature 1..588: note = Bispecific EGFR/c-Met binding domain
  source 1..588: mol_type = other DNA
ttgccagcgccgaagaacctggtagttagcgaggttactgaggacagcgcgcgtctgagctgggacgatccgtgggcgttctacgagagctttctgatccagtatcaagagagcgagaaagtcggtgaagcgattgtgctgaccgtcccgggctccgagcgttcctacgacctgaccggtttgaagccgggtaccgagtatacggtgagcatctacggtgttcacaatgtctataaggacactaatatccgcggtctgcctctgagcgccattttcaccaccgcaccggcaccggctccggctcctgccccgctgccggctccgaagaacttggtggtgagccgtgttaccgaagatagcgcacgcctgagctggacggcaccggatgcggcgttcgatagcttctggattcgctattttgagtttctgggtagcggtgaggcaattgttctgacggtgccgggctctgaacgctcctacgatttgaccggtctgaaaccgggcaccgagtatgtggtgaacattctgagcgttaagggcggtagcatcagcccaccgctgagcgcgatcttcacgactggtggttgc

SEQ ID NO: 167 - DNA, 579 bp, synthetic construct
  misc_feature 1..579: note = Bispecific EGFR/c-Met binding domain
  source 1..579: mol_type = other DNA
ctgccagcccctaagaatctggtcgtgagcgaagtaaccgaggacagcgcccgcctgagctgggacgacccgtgggcgttctatgagtctttcctgattcagtatcaagaaagcgaaaaagttggcgaagcgatcgtcctgaccgtcccgggtagcgagcgctcctacgatctgaccggcctgaaaccgggtacggagtacacggtgtccatttacggtgttcacaatgtgtataaagacaccaacatgcgtggcctgccgctgtcggcgattttcaccaccgcgcctgcgccagcgcctgcaccggctccgctgccggcaccgaagaacctggttgtcagccgtgtgaccgaggatagcgcacgtttgagctggaccgctccggatgcagcctttgacagcttctggattcgttactttgaatttctgggtagcggtgaggcgatcgttctgacggtgccgggctctgaacgcagctatgatttgacgggcctgaagccgggtactgagtacgtggttaacatcatgggcgttaagggtggtaaaatcagcccgccattgtccgcgatctttaccacg

SEQ ID NO: 168 - DNA, 282 bp, synthetic construct
  misc_feature 1..282: note = EGFR binding FN3 domain from ECB97
  source 1..282: mol_type = other DNA
ttgccagcgccgaagaacctggtagttagcgaggttactgaggacagcgcgcgtctgagctgggacgatccgtgggcgttctacgagagctttctgatccagtatcaagagagcgagaaagtcggtgaagcgattgtgctgaccgtcccgggctccgagcgttcctacgacctgaccggtttgaagccgggtaccgagtatacggtgagcatctacggtgttcacaatgtctataaggacactaatatccgcggtctgcctctgagcgccattttcaccacc

SEQ ID NO: 169 - DNA, 282 bp, synthetic construct
  misc_feature 1..282: note = EGFR binding FN3 domain from ECB15
  source 1..282: mol_type = other DNA
ctgccagcccctaagaatctggtcgtgagcgaagtaaccgaggacagcgcccgcctgagctgggacgacccgtgggcgttctatgagtctttcctgattcagtatcaagaaagcgaaaaagttggcgaagcgatcgtcctgaccgtcccgggtagcgagcgctcctacgatctgaccggcctgaaaccgggtacggagtacacggtgtccatttacggtgttcacaatgtgtataaagacaccaacatgcgtggcctgccgctgtcggcgattttcaccacc

SEQ ID NO: 170 - protein, 195 aa, synthetic construct
  REGION 1..195: note = Bispecific EGFR/c-Met binding molecule
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILGVKGGKISPPLSAIFTTC

SEQ ID NO: 171 - protein, 195 aa, synthetic construct
  REGION 1..195: note = Bispecific EGFR/c-Met binding molecule
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVGSGEAIVLTVPGSERSYDLTGLKPGTEYVVNILGVKGGSISPPLSAIFTTC

SEQ ID NO: 172 - protein, 195 aa, synthetic construct
  REGION 1..195: note = Bispecific EGFR/c-Met binding molecule
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVSKGDAIVLTVPGSERSYDLTGLKPGTEYVVNILGVKGGSISPPLSAIFTTC

SEQ ID NO: 173 - protein, 195 aa, synthetic construct
  REGION 1..195: note = Bispecific EGFR/c-Met binding molecule
MLPAPKNLVVSEVTEDSARLSWDDPWAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILSVKGGSISPPLSAIFTTC

SEQ ID NO: 174 - protein, 195 aa, synthetic construct
  REGION 1..195: note = Bispecific EGFR/c-Met binding molecule
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILGVKGGKISPPLSAIFTTC

SEQ ID NO: 175 - protein, 195 aa, synthetic construct
  REGION 1..195: note = Bispecific EGFR/c-Met binding molecule
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVGSGEAIVLTVPGSERSYDLTGLKPGTEYVVNILGVKGGSISPPLSAIFTTC

SEQ ID NO: 176 - protein, 195 aa, synthetic construct
  REGION 1..195: note = Bispecific EGFR/c-Met binding molecule
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFVSKGDAIVLTVPGSERSYDLTGLKPGTEYVVNILGVKGGSISPPLSAIFTTC

SEQ ID NO: 177 - protein, 195 aa, synthetic construct
  REGION 1..195: note = Bispecific EGFR/c-Met binding molecule
MLPAPKNLVVSEVTEDSARLSWDDPHAFYESFLIQYQESEKVGEAIVLTVPGSERSYDLTGLKPGTEYTVSIYGVHNVYKDTNIRGLPLSAIFTTAPAPAPAPAPLPAPKNLVVSRVTEDSARLSWTAPDAAFDSFWIRYFEFLGSGEAIVLTVPGSERSYDLTGLKPGTEYVVNILSVKGGSISPPLSAIFTTC

SEQ ID NO: 178 - protein, 195 aa, synthetic construct; identical to SEQ ID NO: 173
  REGION 1..195: note = Bispecific EGFR/c-Met binding molecule

SEQ ID NO: 179 - protein, 12 aa, synthetic construct
  REGION 1..12: note = Consensus FG loop of EGFR bindiing FN3 domain
  VARIANT 9: note = Xaa may be Met or Ile
HNVYKDTNXRGL

SEQ ID NO: 180 - protein, 12 aa, synthetic construct
  REGION 1..12: note = GF loop of EGFR binding FN3 domain
LGSYVFEHDVML

SEQ ID NO: 181 - skipped

SEQ ID NO: 182 - protein, 95 aa, synthetic construct
  REGION 1..95: note = EGFR binding FN3 domain consensus sequence
  VARIANT 22: note = Xaa may be Ala, Thr, Gly or Asp
  VARIANT 23: note = Xaa may be Ala, Asp, Tyr or Trp
  VARIANT 24: note = Xaa may be Pro, Asp or Asn
  VARIANT 25: note = Xaa may be Leu or absent
  VARIANT 26: note = Xaa may be Asp, His, Arg, Gly, Tyr or Trp
  VARIANT 27: note = Xaa may be Gly, Asp or Ala
  VARIANT 28: note = Xaa may be Ala, Phe, Gly, His or Asp
  VARIANT 29: note = Xaa may be Tyr, Phe or Leu
  VARIANT 84: note = Xaa may be Met or Ile
LPAPKNLVVSEVTEDSLRLSWXXXXXXXXDSFLIQYQESEKVGEAINLTVPGSERSYDLTGLKPGTEYTVSIYGVHNVYKDTNXRGLPLSAEFTT

SEQ ID NO: 183 - protein, 95 aa, synthetic construct
  REGION 1..95: note = EGFR binding FN3 domain consensus sequence
  VARIANT 22: note = Xaa may be Ala, Thr, Gly or Asp
  VARIANT 23: note = Xaa may be Ala, Asp, Tyr or Trp
  VARIANT 24: note = Xaa may be Pro, Asp or Asn
  VARIANT 25: note = Xaa may be Leu or absent
  VARIANT 26: note = Xaa may be Asp, His, Arg, Gly, Tyr or Trp
  VARIANT 27: note = Xaa may be Gly, Asp or Ala
  VARIANT 28: note = Xaa may be Ala, Phe, Gly, His or Asp
  VARIANT 29: note = Xaa may be Tyr, Phe or Leu
LPAPKNLVVSEVTEDSLRLSWXXXXXXXXDSFLIQYQESEKVGEAINLTVPGSERSYDLTGLKPGTEYTVSIYGVLGSYVFEHDVMLPLSAEFTT

SEQ ID NO: 184 - protein, 15 aa, synthetic construct
  REGION 1..15: note = c-Met binding FN3 domain C strand and CD loop consensus sequence
  VARIANT 4: note = Xaa is Trp, Phe or Val
  VARIANT 8: note = Xaa is Asp, Phe or Leu
  VARIANT 10: note = Xaa is Val, Phe or Leu
  VARIANT 11: note = Xaa is Val, Leu or Thr
  VARIANT 12: note = Xaa is Val, Arg, Gly, Leu, Thr or Ser
  VARIANT 13: note = Xaa is Gly, Ser, Ala, Thr or Lys
  VARIANT 15: note = Xaa is Glu or Asp
DSFXIRYXEXXXXGX

SEQ ID NO: 185 - protein, 17 aa, synthetic construct
  REGION 1..17: note = c-Met binding FN3 domain F strand and FG loop consensus sequence
  VARIANT 4: note = Xaa is Tyr, Trp, Ile, Val, Gly or Ala
  VARIANT 6: note = Xaa is Asn, Thr, Qln or Gly
  VARIANT 8: note = Xaa is Leu, Met, Asn or Ile
  VARIANT 9: note = Xaa is Gly or Ser
  VARIANT 14: note = Xaa is Ser, Leu, Gly, Tyr, Thr, Arg, His or Lys
  VARIANT 15: note = Xaa is Ile, Val or Leu
  VARIANT 17: note = Xaa is Val, Thr, His, Ile, Pro, Tyr or Leu
TEYXVXIXXVKGGXXSX

SEQ ID NO: 186 - protein, 89 aa, synthetic construct
  REGION 1..89: note = c-Met binding FN3 domain consensus sequence
  VARIANT 32: note = Xaa is Trp, Phe or Val
  VARIANT 36: note = Xaa is Asp, Phe or Leu
  VARIANT 38: note = Xaa is Val, Phe or Leu
  VARIANT 39: note = Xaa is Val, Leu or Thr
  VARIANT 40: note = Xaa is Val, Arg, Gly, Leu, Thr or Ser
  VARIANT 41: note = Xaa is Gly, Ser, Ala, Thr or Lys
  VARIANT 43: note = Xaa is Glu or Asp
  VARIANT 68: note = Xaa is Tyr, Trp, Ile, Val, Gly or Ala
  VARIANT 70: note = Xaa is Asn, Thr, Qln or Gly
  VARIANT 72: note = Xaa is Leu, Met, Asn or Ile
  VARIANT 73: note = Xaa is Gly or Ser
  VARIANT 78: note = Xaa is Ser, Leu, Gly, Tyr, Thr, Arg, His or Lys
  VARIANT 79: note = Xaa is Ile, Val or Leu
  VARIANT 81: note = Xaa is Val, Thr, His, Ile, Pro, Tyr or Leu
LPAPKNLVVSRVTEDSARLSWTAPDAAFDSFXIRYXEXXXXGXAIVLTVPGSERSYDLTGLKPGTEYXVXIXXVKGGXXSXPLSAEFTT

SEQ ID NO: 187 - protein, 94 aa, synthetic construct
  REGION 1..94: note = EGFR consensus FN3 domain of bispecific EGFR/c-Met molecule
  VARIANT 11: note = Xaa is Glu, Asn or Arg
  VARIANT 14: note = Xaa is Glu or Pro
  VARIANT 17: note = Xaa is Leu or Ala
  VARIANT 25: note = Xaa is His or Trp
  VARIANT 29: note = Xaa is Glu or Asp
  VARIANT 37: note = Xaa is Glu or Pro
  VARIANT 46: note = Xaa is Asn or Val
  VARIANT 73: note = Xaa is Gly or Tyr
  VARIANT 83: note = Xaa is Met or Ile
  VARIANT 91: note = Xaa is Glu or Ile
LPAPKNLVVSXVTXDSXRLSWDDPXAFYXSFLIQYQXSEKVGEAIXLTVPGSERSYDLTGLKPGTEYTVSIYXVHNVYKDTNXRGLPLSAXFTT

SEQ ID NO: 188 - protein, 89 aa, synthetic construct
  REGION 1..89: note = c-Met consensus FN3 domain of bispecific EGFR/c-Met molecule
  VARIANT 11: note = Xaa is Glu, Asn or Arg
  VARIANT 14: note = Xaa is Glu or Pro
  VARIANT 17: note = Xaa is Leu or Ala
  VARIANT 37: note = Xaa is Glu or Pro
  VARIANT 39: note = Xaa is Val or Leu
  VARIANT 40: note = Xaa is Gly or Ser
  VARIANT 41: note = Xaa is Ser or Lys
  VARIANT 43: note = Xaa is Glu or Asp
  VARIANT 46: note = Xaa is Asn or Val
  VARIANT 72: note = Xaa is Leu or Met
  VARIANT 73: note = Xaa is Gly or Ser
  VARIANT 78: note = Xaa is Ser or Lys
  VARIANT 86: note = Xaa is Glu or Ile
LPAPKNLVVSXVTXDSXRLSWTAPDAAFDSFWIRYFXFXXXGXAIXLTVPGSERSYDLTGLKPGTEYVVNIXXVKGGXISPPLSAXFTT

SEQ ID NO: 189 - protein, 125 aa, Homo sapiens [sequence omitted]

SEQ ID NO: 190 - protein, 107 aa, Homo sapiens [sequence omitted]

SEQ ID NO: 191 - protein, 121 aa, Homo sapiens
EVQLVESGGGLVQPGGSLRLSCAASGFTFSSYWMNWVRQAPGKGLEWVANIKKDGSEKYYVDSVKGRFTISRDNAKNSLYLQMNSLRAEDTAVYYCARDLGWGWGWYFDLWGRGTLVTVSS

SEQ ID NO: 192 - protein, 107 aa, Homo sapiens [sequence omitted]

SEQ ID NO: 193 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 194 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 195 - protein, 125 aa, Homo sapiens
EVQLVESGGGLVKPGGSLKLSCAASGFTFSDYYMYWVRQTPEKRLEWVATISDDGSYTYYPDSVKGRFTISRDNAKNNLYLQMSSLKSEDTAMYYCAREGLYYYGSGSYYNQDYWGQGTLVTVSS

SEQ ID NO: 196 - protein, 106 aa, Homo sapiens
QLTQSPSSLSASVGDRVTITCRASQGLSSALAWYRQKPGKAPKLLIYDASSLESGVPSRFSGSGSGTDFTLTISSLQPEDFATYYCQQFTSYPQITFGQGTRLEIK

SEQ ID NO: 197 - protein, 457 aa, Homo sapiens
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 198 - protein, 457 aa, Homo sapiens
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 199 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 200 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 202 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 204 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 205 - DNA, 1368 bp, Homo sapiens
  source 1..1368: mol_type = unassigned DNA
caggtgcagctggtcgagagcggcggaggggtggtgcagcccggcagaagcctgaggctgtcctgcgccgccagcggcttcaccttcagcacctacggcatgcactgggtgcggcaggccccaggcaagggcctggagtgggtggccgtgatctgggacgacggcagctacaagtactacggcgacagcgtgaagggcaggttcaccatcagcagggacaacagcaagaacaccctgtacctgcagatgaacagcctgagggccgaggacaccgccgtgtactactgcgccagggacggcatcaccatggtgcggggcgtgatgaaggactacttcgactactggggccagggcaccctggtgaccgtgagcagcgccagcaccaagggcccaagcgtgttccccctggcccccagcagcaagagcaccagcggcggcacagccgccctgggctgcctggtgaaggactacttccccgagccagtgaccgtgtcctggaactctggcgccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggacccagcgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgccccccagccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcctcctgtacagcaagctgaccgtggacaagtccaggtggcagcagggcaacgtgttcagctgcagcgtgatgcacgaggccctgcacaaccactacacccagaagtccctgagcctgagccccggcaaatga

SEQ ID NO: 206 - DNA, 642 bp, Homo sapiens
  source 1..642: mol_type = unassigned DNA
atccagctgacccagagccccagcagcctgagcgccagcgtgggcgaccgggtgaccatcacctgccgggccagccaggacatcagcagcgccctggtctggtatcagcagaagcccggcaaggcccccaagctgctgatctacgacgccagctccctggaaagcggcgtgcccagccggttcagcggcagcgagagcggcaccgacttcaccctgaccatcagcagcctgcagcccgaggacttcgccacctactactgccagcagttcaacagctaccccctgacctttggcggcggaacaaaggtggagatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgtccaaggccgactacgagaagcacaaggtgtacgcctgcgaggtgacccaccagggcctgtccagccccgtgaccaagagcttcaacaggggcgagtgctga

SEQ ID NO: 207 - DNA, 1350 bp, Homo sapiens
  source 1..1350: mol_type = unassigned DNA
caggttcagctggtgcagtctggagctgaggtgaagaagcctggggcctcagtgaaggtctcctgcgagacttctggttacacctttaccagctatggtatcagctgggtgcgacaggcccctggacacgggcttgagtggatgggatggatcagcgcttacaatggttacacaaactatgcacagaagctccagggcagggtcaccatgaccacagacacatccacgagcacagcctacatggagctgaggagcctgagatctgacgacacggccgtgtattactgtgcgagagatctgagaggaactaactactttgactactggggccagggaaccctggtcaccgtctcctcagcctccaccaagggcccaagcgtgttccctctggcccccagcagcaagagcacatctggcggaacagccgccctgggctgcctggtgaaagactacttccccgagcccgtgaccgtgtcctggaactctggcgccctgaccagcggcgtgcacacctttccagccgtgctgcagagcagcggcctgtacagcctgtccagcgtggtgaccgtgcccagcagctccctgggcacccagacctacatctgcaacgtgaaccacaagcccagcaacaccaaggtggacaagcgggtggaacccaagagctgcgacaagacccacacctgtcccccctgccctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgacagtgctgcaccaggactggctgaacggcaaagagtacaagtgcaaggtctccaacaaggccctgcctgctcccatcgagaaaaccatcagcaaggccaagggccagccccgcgagcctcaggtgtacacactgcctcccagccgggaagagatgaccaagaaccaggtgtccctgacctgtctggtgaaaggcttctaccccagcgatatcgccgtggaatgggagagcaacggacagcccgagaacaactacaagaccaccccccctgtgctggacagcgacggctccttcttcctgtactctcggctgaccgtggacaagagccggtggcagcagggaaacgtgttcagctgcagcgtgatgcacgaggccctgcacaaccactacacccagaagtccctgagcctgagccccgggaagtga

SEQ ID NO: 208 - DNA, 645 bp, Homo sapiens
  source 1..645: mol_type = unassigned DNA
gacatccagatgacccagtccccctcctccgtgtccgcctctgtgggcgacagagtgaccatcacctgtcgggcctcccagggcatctccaactggctggcctggttccagcacaagcccggcaaggcccccaagctgctgatctacgccgcctcctccctgctgtccggcgtgccctccagattctccggctctggctccggcaccgacttcaccctgaccatctccagcctgcagcccgaggacttcgccacctactactgccagcaggccaactccttccccatcaccttcggccagggcacccggctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgtccaaggccgactacgagaagcacaaggtgtacgcctgcgaggtgacccaccagggcctgtccagccccgtgaccaagagcttcaacaggggcgagtgctga

SEQ ID NO: 209 - protein, 215 aa, Homo sapiens
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 5 aa, Homo sapiens
TYGMH

SEQ ID NO: 211 - protein, 17 aa, Homo sapiens
VIWDDGSYKYYGDSVKG

SEQ ID NO: 212 - protein, 16 aa, Homo sapiens
DGITMVRGVMKDYFDY

SEQ ID NO: 213 - protein, 11 aa, Homo sapiens
RASQDISSALV

SEQ ID NO: 214 - protein, 7 aa, Homo sapiens
DASSLES

SEQ ID NO: 215 - protein, 9 aa, Homo sapiens
QQFNSYPLT

SEQ ID NO: 216 - protein, 5 aa, Homo sapiens
SYGIS

SEQ ID NO: 217 - protein, 17 aa, Homo sapiens
WISAYNGYTNYAQKLQG

SEQ ID NO: 218 - protein, 10 aa, Homo sapiens
DLRGTNYFDY

SEQ ID NO: 219 - protein, 11 aa, Homo sapiens
RASQGISNWLA

SEQ ID NO: 220 - protein, 7 aa, Homo sapiens
AASSLLS

SEQ ID NO: 221 - protein, 9 aa, Homo sapiens
QQANSFPIT

SEQ ID NO: 222 - protein, 5 aa, Homo sapiens
SYWMN

SEQ ID NO: 223 - protein, 17 aa, Homo sapiens
NIKKDGSEKYYVDSVKG

SEQ ID NO: 224 - protein, 12 aa, Homo sapiens
DLGWGWGWYFDL

SEQ ID NO: 225 - protein, 11 aa, Homo sapiens
RASQSVSSYLA

SEQ ID NO: 226 - protein, 7 aa, Homo sapiens
DASNRAT

SEQ ID NO: 227 - protein, 9 aa, Homo sapiens
QQRSNWPPT

SEQ ID NO: 228 - protein, 5 aa, Homo sapiens
DYYMY

SEQ ID NO: 229 - protein, 17 aa, Homo sapiens
TISDDGSYTYYPDSVKG

SEQ ID NO: 230 - protein, 16 aa, Homo sapiens
EGLYYYGSGSYYNQDY

SEQ ID NO: 231 - protein, 11 aa, Homo sapiens
RASQGLSSALA

SEQ ID NO: 232 - protein, 7 aa, Homo sapiens
DASSLES

SEQ ID NO: 233 - protein, 10 aa, Homo sapiens
QQFTSYPQIT

SEQ ID NO: 234 - protein, 451 aa, Homo sapiens
EVQLVESGGGLVQPGGSLRLSCAASGFTFSSYWMNWVRQAPGKGLEWVANIKKDGSEKYYVDSVKGRFTISRDNAKNSLYLQMNSLRAEDTAVYYCARDLGWGWGWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 235 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 455 aa, Homo sapiens
EVQLVESGGGLVKPGGSLKLSCAASGFTFSDYYMYWVRQTPEKRLEWVATISDDGSYTYYPDSVKGRFTISRDNAKNNLYLQMSSLKSEDTAMYYCAREGLYYYGSGSYYNQDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 237 - protein, 213 aa, Homo sapiens
QLTQSPSSLSASVGDRVTITCRASQGLSSALAWYRQKPGKAPKLLIYDASSLESGVPSRFSGSGSGTDFTLTISSLQPEDFATYYCQQFTSYPQITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 15 aa, Homo sapiens
PEFRDSYPIKYVHAF

SEQ ID NO: 239 - protein, 16 aa, Homo sapiens
FAQSKPDSAEPMDRSA

SEQ ID NO: 240 - protein, 16 aa, Homo sapiens
PGAQLARQIGASLNDD